(19) United States
(12) Patent Application Publication
(10) Pub. No.: US 2023/0192818 A1
(43) Pub. Date: Jun. 22, 2023

(54) SARS-COV-2 ANTIBODIES AND RELATED COMPOSITIONS AND METHODS OF USE

(71) Applicant: Twist Bioscience Corporation, South San Francisco, CA (US)

(72) Inventors: Aaron SATO, Burlingame, CA (US); Qiang LIU, Palo Alto, CA (US); Tom YUAN, San Francisco, CA (US); Ana G. LUJAN HERNANDEZ, San Francisco, CA (US)

(21) Appl. No.: 17/820,536

(22) Filed: Aug. 17, 2022

Related U.S. Application Data
(60) Provisional application No. 63/234,187, filed on Aug. 17, 2021.

(51) Int. Cl. C07K 16/10; A61P 31/14

(52) U.S. Cl. CPC C07K 16/10; A61P 31/14; A61K 2039/505

(57) ABSTRACT

Provided herein are methods and compositions relating to libraries of optimized antibodies having nucleic acids encoding for an antibody comprising modified sequences. Libraries described herein comprise nucleic acids encoding SARS-CoV-2 or ACE2 antibodies. Further described herein are protein libraries generated when the nucleic acid libraries are translated. Further described herein are cell libraries expressing variegated nucleic acid libraries described herein.

### CROSS REFERENCE

This application claims the benefit of U.S. Provisional Patent Application No. 63/234,187, filed on Aug. 17, 2021, which is incorporated by reference in its entirety.

### SEQUENCE LISTING

The instant application contains a Sequence Listing which has been submitted electronically in ASCII formal and is hereby incorporated by reference in its entirety. Said ASCII copy, created on Dec. 28, 2022, is named 44854-837_201_SL.xml and is 660,000 bytes in size.

### BACKGROUND

Coronaviruses like severe acute respiratory coronavirus 2 (SARS-CoV-2) can cause severe respiratory problems. Therapies are needed for treating and preventing viral infection caused by coronaviruses like SARS-CoV-2. Antibodies possess the capability to bind with high specificity and affinity to biological targets. However, the design of therapeutic antibodies is challenging due to balancing of immunological effects with efficacy. Thus, there is a need to develop compositions and methods for the optimization of antibody properties in order to develop effective therapies for treating coronavirus infections.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

In certain aspects, disclosed herein is an antibody or antibody fragment comprising a variable domain, heavy chain region (VH) and a variable domain, light chain region (VL), wherein the VH region comprises complementarity determining regions CDRH1, CDRH2, and CDRH3, and wherein (a) an amino acid sequence of CDRH1 is as set forth in any one of SEQ ID NOs: 1-89; (b) an amino acid sequence of CDRH2 is as set forth in any one of SEQ ID NOs: 90-178; (c) an amino acid sequence of CDRH3 is as set forth in any one of SEQ ID NOs: 179-267, and wherein the VL region comprise comprises complementarity determining regions CDRL1, CDRL2, and CDRL3, and wherein (a) an amino acid sequence of CDRL1 is as set forth in any one of SEQ ID NOs: 268-356; (b) an amino acid sequence of CDRL2 is as set forth in any one of SEQ ID NOs: 357-445; (c) an amino acid sequence of CDRL3 is as set forth in any one of SEQ ID NOs: 446-534. In some embodiments, the antibody or antibody fragment binds to a spike glycoprotein. In some embodiments, the antibody or antibody fragment binds to a receptor binding domain of the spike glycoprotein. In some embodiments, the antibody or antibody fragment comprises a K_{D} of less than 50 nM. In some embodiments, the antibody or antibody fragment comprises a K_{D} of less than 25 nM. In some embodiments, the antibody or antibody fragment comprises a K_{D} of less than 10 nM. In some embodiments, the antibody or antibody fragment comprises a K_{D} of less than 5 nM. In some embodiments, the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. In some embodiments, the antibody is a single domain antibody.

In certain aspects, disclosed herein is an antibody or antibody fragment comprising a variable domain, heavy chain region (VH) comprising an amino acid sequence at least about 90%/9 identical to a sequence as set forth in any one of SEQ ID NOs: 535-623 and a variable domain, light chain region (VL) comprising an amino acid sequence at least about 90% identical to a sequence as set forth in any one of SEQ ID NOs: 624-712. In some embodiments, the antibody or antibody fragment binds to a spike glycoprotein. In some embodiments, the antibody or antibody fragment binds to a receptor binding domain of the spike glycoprotein. In some embodiments, the antibody or antibody fragment comprises a K_{D} of less than 50 nM. In some embodiments, the antibody or antibody fragment comprises a K_{D} of less than 25 nM. In some embodiments, the antibody or antibody fragment comprises a K_{D} of less than 10 nM. In some embodiments, the antibody or antibody fragment comprises a K_{D} of less than 5 nM. In some embodiments, the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof. In some embodiments, the antibody is a single domain antibody.

In certain aspects, disclosed herein is a nucleic acid composition comprising: a) a first nucleic acid encoding a variable domain, heavy chain region (VH) comprising an amino acid sequence at least about 90% identical to a sequence as set forth in any one of SEQ ID NOs: 535-623; b) a second nucleic acid encoding a variable domain, light chain region (VL) comprising at least about 90% identical to a sequence as set forth in any one of SEQ ID NOs: 624-712; and an excipient.

In certain aspects disclosed herein is an antibody or antibody fragment comprising an amino acid sequence at least about 90% identical to a sequence as set forth in SEQ ID NO: 713. In some embodiments, the antibody or antibody fragment binds to a spike glycoprotein. In some embodiments, the antibody or antibody fragment binds to a receptor binding domain of the spike glycoprotein. In some embodiments, the antibody or antibody fragment comprises a K_{D} of less than 50 nM. In some embodiments, the antibody or antibody fragment comprises a K_{D} of less than 25 nM. In some embodiments, the antibody or antibody fragment comprises a K_{D} of less than 10 nM. In some embodiments, the antibody or antibody fragment comprises a K_{D} of less than 5 nM. In some embodiments, the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

In certain aspects, disclosed herein is a method of treating a SARS-CoV-2 infection, comprising administering the antibody or antibody fragment described herein. In some embodiments, the antibody is administered prior to exposure to SARS-CoV-2. In some embodiments, the antibody is administered at least about 1 week prior to exposure to SARS-CoV-2. In some embodiments, the antibody is administered at least about 1 month prior to exposure to SARS-CoV-2. In some embodiments, the antibody is administered at least about 5 months prior to exposure to SARS-CoV-2. In some embodiments, the antibody is administered after exposure to SARS-CoV-2. In some embodiments, the antibody is administered at most about 24 hours after exposure to SARS-CoV-2. In some embodiments, the antibody is administered at most about 1 week after exposure to SARS-CoV-2. In some embodiments, the antibody is administered at most about 1 month after exposure to SARS-CoV-2.

In certain aspects, disclosed herein is a method of treating an individual with a SARS-CoV-2 infection with the antibody or antibody fragment described herein comprising: obtaining or having obtained a sample from the individual; performing or having performed an expression level assay on the sample to determine expression levels of SARS-CoV-2 antibodies; and if the sample has an expression level of the SARS-CoV-2 antibodies then administering to the individual the antibody or antibody fragment described herein, thereby treating the SARS-CoV-2 infection. In certain aspects, disclosed herein is a method for optimizing an antibody comprising: providing a plurality of polynucleotide sequences encoding for an antibody or antibody fragment, wherein the antibody or antibody fragment is derived from a subject having SARS-CoV-2; generating a nucleic acid library comprising the plurality of sequences that when translated encode for antibodies or antibody fragments that bind SARS-CoV-2 or ACE2 protein, wherein each of the sequences comprises a predetermined number of variants within a CDR relative to an input sequence that encodes an antibody; wherein the library comprises at least 50,000 variant sequences; and synthesizing the at least 50,000 variant sequences. In some embodiments, the antibody library comprises at least 100,000 sequences. In some embodiments, the method further comprises enriching a subset of the variant sequences. In some embodiments, the method further comprises expressing the antibody or antibody fragments corresponding to the variant sequences. In some embodiments, the polynucleotide sequence is a murine, human, or chimeric antibody sequence. In some embodiments, each sequence of the plurality of variant sequences comprises at least one variant in each CDR of a heavy chain or light chain, relative to the input sequence. In some embodiments, each sequence of the plurality of variant sequences comprises at least two variants in each CDR of a heavy chain or light chain relative to the input sequence. In some embodiments, at least one sequence when translated encodes for an antibody or antibody fragment having at least 5×higher binding affinity than a binding affinity of the input sequence. In some embodiments, at least one sequence when translated encodes for an antibody or antibody fragment having at least 25× higher binding affinity than a binding affinity of the input sequence. In some embodiments, at least one sequence when translated encodes for an antibody or antibody fragment having at least 50× higher binding affinity than a binding affinity of the input sequence. In some embodiments, each sequence comprises at least one variant in each CDR of a heavy chain or light chain relative to a germline sequence of the input sequence. In some embodiments, the nucleic acid library has a theoretical diversity of at least 10¹⁰ sequences. In some embodiments, the nucleic acid library has a theoretical diversity of at least 10¹² sequences.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a workflow for antibody optimization.

FIG. 2 presents a diagram of steps demonstrating an exemplary process workflow for gene synthesis as disclosed herein.

FIG. 3 illustrates an example of a computer system.

FIG. 4 is a block diagram illustrating an architecture of a computer system.

FIG. 5 is a diagram demonstrating a network configured to incorporate a plurality of computer systems, a plurality of cell phones and personal data assistants, and Network Attached Storage (NAS).

FIG. 6 is a block diagram of a multiprocessor computer system using a shared virtual address memory space.

FIG. 7 depicts the locations of different mutations in SARS-CoV-2 variants.

FIGS. 8A-8B are schemas of a panning workflow.

FIG. 9A depicts Carterra SPR kinetics against the SARS-COV-2 S1.

FIG. 9B depicts Carterra SPR kinetics against the SARS-CoV-2 501.V2 S1.

FIG. 9C depicts Carterra SPR kinetics against the SARS-CoV-2 B.1.1.7 S1.

FIG. 9D depicts Carterra SPR kinetics against the SARS-COV-2 CA Var. W152C L452R D614G S1.

FIG. 9E depicts Carterra SPR kinetics against the SARS-COV-2 RBD India Var. L452R E484Q S1.

FIG. 10 depicts the S1-RBD-mFc binding competition assay used.

FIG. 11A depicts the results of the competition assay against Acm S1.

FIG. 11B depicts the results of the competition assay against D614G S1.

FIG. 11C depicts the results of the competition assay against 501.V2 South Africa S1.

FIG. 11D depicts the results of the competition assay against B.1.1.7 UK S1.

FIG. 12 depicts the results of an Acro S1-mFc binding competition assay comparing antibody 181-8 mutant fc, 15-3_fc_mutant and Acro neutralizing antibody.

FIG. 13A depicts the binding of the CA variant S1 to Vern cells.

FIG. 13B depicts the results of a competition assay of the panel of variants against the CCA S1 spike protein.

FIGS. 14A-14E depict the result of a binding competition assay comparing SARS-CoV-2 cross-reacting antibody variants with different strains of SARS-CoV-2 including Acro S1 (FIG. 14A), D614G Variant (FIG. 14B), South Africa Variant 501.V2 (FIG. 14C), UK Variant B.1.1.7 (FIG. 14D). California Variant W52C_L452R_D614G (FIG. 14E).

FIGS. 15A-15B depict the general structure of Antibody 813 (FIG. 15A) and another schematic of the structure of antibody 813 (FIG. 15B). FIG. 15C shows the results of alanine scanning mutagenesis of Antibody 813 to identify critical residues for each VHH binding specificity (starred residues indicate critical binding residues of Antibody 15-3; unstarred residues indicate critical binding residues of Antibody 3-31). FIGS. 15D-15F show the epitope of Antibody 813 in context of an ACE2 binding site from angle 1 (FIG. 15D), angle 2 (FIG. 15E) and angle 3 (FIG. 15F). In FIGS. 15D-15F, red denotes critical binding residues of parental Antibody 15-3 (at the N-terminal VHH of Antibody 813); green denotes critical binding residues of parental Antibody 3-31 (at the C-terminal VHH of Antibody 813); dark grey indicates the ACE2 binding interface; light grey indicates the receptor binding domain (RBD), blue indicates the ACE2 helix; and yellow indicates the RBD helix.

FIGS. 16A-16D depict the structural definition of Antibody 813 with the Ancestral Spike trimer as determined by CryoEM from the side (FIG. 16A), from the front (FIG. 16B), from the back (FIG. 16C), and from the top (FIG. 16D). Magenta/Red/Green represent monomers of the spike trimer, further denoted as chain A (magenta), chain B (red), and chain C (green). Grey density represents the bispecific antibody constant fragment. VHH1 is depicted in gold, Vhh2 is blue, and VHH3 is orange. FIG. 16E lists spike residues in explicit bonds for each of the three distinct binding epitopes (VHH1, VHH2, and VHH3), while highlighted residues represent the critical contacts identified in alanine scanning mutagenesis. FIGS. 16F-16H show reconstructed low-resolution maps of negative staining showing “additional density” (denoted by the boxes) beyond the spike trimer consistent with multi-valent binding interactions of Antibody 813 using the Ancestral variant (FIG. 16F), the Delta variant (FIG. 16G), and the Omicron variant (FIG. 16H).

FIGS. 17A-17D show functionality cell-based assays for phagocytosis (FIG. 17A), antibody-dependent cell-mediated cytotoxicity (ADCC) (FIG. 17B), complement-dependent cytotoxicity (CDC)(FIG. 17C), and antibody-dependent enhancement (ADE)(FIG. 17D).

FIG. 18A shows representative inhibition graphs for pseudovirus neutralization assays on Ancestral SARS-CoV-2 (Panel A), and several variants of concern including B.1.1.529 (Panel B), BA2.12.1 (Panel C), and BA.4 (Panel D). FIG. 18B shows a summary of all data collected in neutralization experiments.

FIG. 19A shows an overlay plot showing dose-dependent inhibition curves for authentic virus neutralization assay using Vero E6+TMPRSS2 cells. FIG. 19B shows overlay plots showing dose-dependent inhibition curves for authentic virus neutralization assays on different cell types.

FIG. 20A shows dose-dependent inhibition by Antibody 813 of ACE2/spike protein interactions with the Ancestral variant (Panel A), the D614 variant (Panel B), the Delta variant (Panel C), and the Omicron variant (Panel D). FIG. 20B shows a summary of the data for the ACE2 blockade experiments.

FIG. 21A shows that Antibody 813 and the recombinant higG1 control show near-identical dose-dependent binding to C1q. FIG. 21B shows high affinity FCGR1 interactions while FIG. 21C shows other Pc receptor interactions.

FIGS. 22A-22C depict the results of escape assays. FIG. 22A shows neutralization curves for the rVSV-SARS-CoV-2 ancestral variant. FIG. 22B shows escape assay results from the Antibody 15 parent. FIG. 22C shows escape assay results from Antibody 813.

FIG. 23A shows in vivo safety data for non-clinical rat studies. FIG. 23B shows a summary table of calculated PK, AUC, EM and other data. FIG. 23C shows serum concentration kinetics curves for a single 30 mg/kg IV dose in rats. FIG. 23D shows serum concentration kinetics curves for a single 30 mg/kg subcutaneous dose in rats.

FIG. 24 depicts a schematic for an Antibody 813 dosing scheme.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence. cDNA described herein may be generated by de novo synthesis.

Antibody Optimization Library for Coronavirus

Provided herein are methods, compositions, and systems for the optimization of antibodies for coronavirus. In some embodiments, the antibodies are optimized for SARS-CoV, MERS-CoV, CoV-229F, HCoV-NL63, HCoV-OC43, or HCoV-HKU1. In some embodiments, the antibodies are optimized for SARS-CoV-2. In some embodiments, the antibodies are optimized for a receptor that binds to the coronavirus. In some embodiments, the receptor of the coronavirus is ACE2 or dipeptidyl peptidase 4 (DPP4). In some embodiments, the antibodies are optimized based on interactions between the coronavirus and the receptor that binds the coronavirus. In some embodiments, the antibodies are optimized for angiotensin-converting enzyme 2 (ACE2). In some embodiments, the antibodies are optimized based on interactions between SARS-CoV-2 and ACE2.

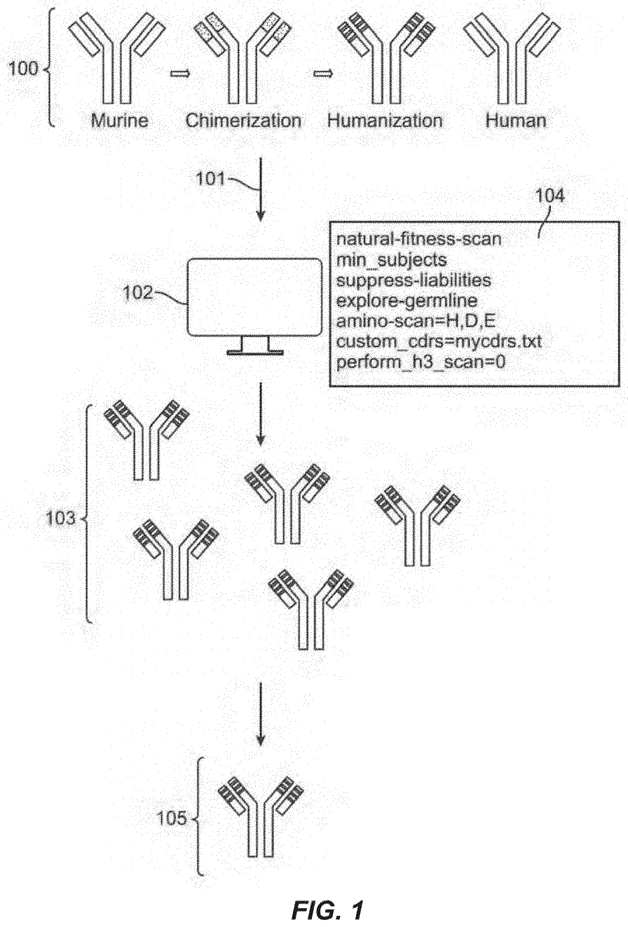

Antibodies are in some instances optimized by the design of in-silico libraries comprising variant sequences of an input antibody sequence (FIG. 1). Input sequences 100 are in some instances modified in-silico 102 with one or more mutations or variants to generate libraries of optimized sequences 103. In some instances, such libraries are synthesized, cloned into expression vectors, and translation products (antibodies) evaluated for activity. In some instances, fragments of sequences are synthesized and subsequently assembled. In some instances, expression vectors are used to display and enrich desired antibodies, such as phage display. Selection pressures used during enrichment in some instances includes, but is not limited to, binding affinity, toxicity, immunological tolerance, stability, receptor-ligand competition, or developability. Such expression vectors allow antibodies with specific properties to be selected (“panning”), and subsequent propagation or amplification of such sequences enriches the library with these sequences. Panning rounds can be repeated any number of times, such as 1, 2, 3, 4, 5, 6, 7, or more than 7 rounds. Sequencing at one or more rounds is in some instances used to identify which sequences 105 have been enriched in the library.

Described herein are methods and systems of in-silico library design. For example, an antibody or antibody fragment sequence is used as input. In some instances, the antibody sequence used as input is an antibody or antibody fragment sequence that binds SARS-CoV-2. In some instances, the input is an antibody or antibody fragment sequence that binds a protein of SARS-CoV-2. In some instances, the protein is a spike glycoprotein, a membrane protein, an envelope protein, a nucleocapsid protein, or combinations thereof. In some instances, the protein is a spike glycoprotein of SARS-CoV-2. In some instances, the protein is a receptor binding domain of SARS-CoV-2. In some instances, the input sequence is an antibody or antibody fragment sequence that binds angiotensin-converting enzyme 2 (ACE2). In some instances, the input sequence is an antibody or antibody fragment sequence that binds an extracellular domain of the angiotensin-converting enzyme 2 (ACE2).

A database 102 comprising known mutations or variants of one or more viruses is queried 101, and a library 103 of sequences comprising combinations of these mutations or variants are generated. In some instances, the database comprises known mutations or variants of SARS-CoV-like coronaviruses, SARS-CoV-2, SARS-CoV, or combinations thereof. In some instances, the database comprises known mutations or variants of the spike protein of SARS-CoV-like coronaviruses, SARS-CoV-2. SARS-CoV, or combinations thereof. In some instances, the database comprises known mutations or variants of the receptor binding domain of SARS-CoV-like coronaviruses, SARS-CoV-2, SARS-CoV, or combinations thereof. In some instances, the database comprises mutations or variants of a protein of SARS-CoV-like coronaviruses, SARS-CoV-2, SARS-CoV, or combinations thereof that binds to ACE2.

In some instances, the input sequence is a heavy chain sequence of an antibody or antibody fragment that binds SARS-CoV-like coronaviruses. SARS-CoV-2. SARS-CoV, or combinations thereof. In some instances, the input sequence is a light chain sequence of an antibody or antibody fragment that binds SARS-CoV-like coronaviruses, SARS-CoV-2, SARS-CoV, or combinations thereof. In some instances, the heavy chain sequence comprises varied CDR regions. In some instances, the light chain sequence comprises varied CDR regions. In some instances, known mutations or variants from CDRs are used to build the sequence library. Filters 104, or exclusion criteria, are in some instances used to select specific types of variants for members of the sequence library. For example, sequences having a mutation or variant are added if a minimum number of organisms in the database have the mutation or variant. In some instances, additional CDRs are specified for inclusion in the database. In some instances, specific mutations or variants or combinations of mutations or variants are excluded from the library (e.g., known immunogenic sites, structure sites, etc.). In some instances, specific sites in the input sequence are systematically replaced with histidine, aspartic acid, glutamic acid, or combinations thereof. In some instances, the maximum or minimum number of mutations or variants allowed for each region of an antibody are specified. Mutations or variants in some instances are described relative to the input sequence or the input sequence's corresponding germline sequence. For example, sequences generated by the optimization comprise at least 1, 2, 3, 4, 5, 6, 7, 8, 9, 10, 1, 12, 13, 14, 15, 16, or more than 16 mutations or variants from the input sequence. In some instances, sequences generated by the optimization comprise no more than 1, 2, 3, 4, 5, 6, 7, 8, 9, 10, 11, 12, 13, 14, 15, 16, or no more than 18 mutations or variants from the input sequence. In some instances, sequences generated by the optimization comprise about 1, 2, 3, 4, 5, 6, 7, 8, 9, 10, 11, 12, 13, 14, 15, 16, or about 18 mutations or variants relative to the input sequence. In some instances, sequences generated by the optimization comprise about 1, 2, 3, 4, 5, 6, or 7 mutations or variants from the input sequence in a first CDR region. In some instances, sequences generated by the optimization comprise about 1, 2, 3, 4, 5, 6, or 7 mutations or variants from the input sequence in a second CDR region. In some instances, sequences generated by the optimization comprise about 1, 2, 3, 4, 5, 6, or 7 mutations or variants from the input sequence in a third CDR region. In some instances, sequences generated by the optimization comprise about 1, 2, 3, 4, 5, 6, or 7 mutations or variants from the input sequence in a first CDR region of a heavy chain. In some instances, sequences generated by the optimization comprise about 1, 2, 3, 4, 5, 6, or 7 mutations or variants from the input sequence in a second CDR region of a heavy chain. In some instances, sequences generated by the optimization comprise about 1, 2, 3, 4, 5, 6, or 7 mutations or variants from the input sequence in a third CDR region of a heavy chain. In some instances, sequences generated by the optimization comprise about 1, 2, 3, 4, 5, 6, or 7 mutations or variants from the input sequence in a first CDR region of a light chain. In some instances, sequences generated by the optimization comprise about 1, 2, 3, 4, 5, 6, or 7 mutations or variants from the input sequence in a second CDR region of a light chain. In some instances, sequences generated by the optimization comprise about 1, 2, 3, 4, 5, 6, or 7 mutations or variants from the input sequence in a third CDR region of a light chain. In some instances, a first CDR region is CDR1. In some instances, a second CDR region is CDR2. In some instances, a third CDR region is CDR3. In-silico antibodies libraries are in some instances synthesized, assembled, and enriched for desired sequences.

The germline sequences corresponding to an input sequence may also be modified to generate sequences in a library. For example, sequences generated by the optimization methods described herein comprise at least 1, 2, 3, 4, 5, 6, 7, 8, 9, 10, 1, 12, 13, 14, 15, 16, or more than 16 mutations or variants from the germline sequence. In some instances, sequences generated by the optimization comprise no more than 1, 2, 3, 4, 5.6, 7, 8, 9, 10, 11, 12, 13, 14, 15, 16, or no more than 18 mutations or variants from the germline sequence. In some instances, sequences generated by the optimization comprise about 1, 2, 3, 4, 5, 6, 7, 8, 9, 10, 11, 12, 13, 14, 15, 16, or about 18 mutations or variants relative to the germline sequence.

Provided herein are methods, systems, and compositions for antibody optimization, wherein the input sequence comprises mutations or variants in an antibody region. Exemplary regions of the antibody include, but are not limited to, a complementarity-determining region (CDR), a variable domain, or a constant domain. In some instances, the CDR is CDR1, CDR2, or CDR3. In some instances, the CDR is a heavy domain including, but not limited to, CDRH1, CDRH2, and CDRH3. In some instances, the CDR is a light domain including, but not limited to, CDRL1, CDRL2, and CDRL3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH). In some instances, sequences generated by the optimization comprise about 1, 2, 3, 4, 5, 6, or 7 mutations or variants from the germline sequence in a first CDR region. In some instances, sequences generated by the optimization comprise about 1, 2, 3, 4, 5, 6, or 7 mutations or variants from the germline sequence in a second CDR region. In some instances, sequences generated by the optimization comprise about 1.2, 3, 4, 5, 6, or 7 mutations or variants from the germline sequence in a third CDR region. In some instances, sequences generated by the optimization comprise about 1, 2, 3, 4, 5, 6, or 7 mutations or variants from the germline sequence in a first CDR region of a heavy chain. In some instances, sequences generated by the optimization comprise about 1, 2, 3, 4, 5, 6, or 7 mutations or variants from the germline sequence in a second CDR region of a heavy chain. In some instances, sequences generated by the optimization comprise about 1, 2, 3, 4, 5, 6, or 7 mutations or variants from the germline sequence in a third CDR region of a heavy chain. In some instances, sequences generated by the optimization comprise about 1, 2, 3, 4, 5, 6, or 7 mutations or variants from the germline sequence in a first CDR region of a light chain. In some instances, sequences generated by the optimization comprise about 1, 2, 3, 4, 5, 6, or 7 mutations or variants from the germline sequence in a second CDR region of a light chain. In some instances, sequences generated by the optimization comprise about 1, 2, 3, 4, 5, 6, or 7 mutations or variants from the germline sequence in a third CDR region of a light chain. In some instances, a first CDR region is CDR1. In some instances, a second CDR region is CDR2. In some instances, a third CDR region is CDR3.

VHH Libraries

Provided herein are methods, compositions, and systems for generation of antibodies or antibody fragments. In some instances, the antibodies or antibody fragments are single domain antibodies. Methods, compositions, and systems described herein for the optimization of antibodies comprise a ratio-variant approach that mirror the natural diversity of antibody sequences. In some instances, libraries of optimized antibodies comprise variant antibody sequences. In some instances, the variant antibody sequences are designed comprising variant CDR regions. In some instances, the variant antibody sequences comprising variant CDR regions are generated by shuffling the natural CDR sequences in a llama, humanized, or chimeric framework. In some instances, such libraries are synthesized, cloned into expression vectors, and translation products (antibodies) evaluated for activity. In some instances, fragments of sequences are synthesized and subsequently assembled. In some instances, expression vectors are used to display and enrich desired antibodies, such as phage display. In some instances, the phage vector is a Fab phagemid vector. Selection pressures used during enrichment in some instances includes, but is not limited to, binding affinity, toxicity, immunological tolerance, stability, receptor-ligand competition, or developability. Such expression vectors allow antibodies with specific properties to be selected (“panning”), and subsequent propagation or amplification of such sequences enriches the library with these sequences. Panning rounds can be repeated any number of times, such as 1, 2, 3, 4, 5, 6, 7, or more than 7 rounds. In some instances, each round of panning involves a number of washes. In some instances, each round of panning involves at least or about 1, 2, 3, 4, 5, 6, 7, 8, 9, 10, 11, 12, 13, 14, 15, 16, or more than 16 washes.

Described herein are methods and systems of in-silico library design. Libraries as described herein, in some instances, are designed based on a database comprising a variety of antibody sequences. In some instances, the database comprises a plurality of variant antibody sequences against various targets. In some instances, the database comprises at least 100, 500, 1000, 1500, 2000, 2500, 3000, 3500, 4000, 4500, 5000, or more than 5000 antibody sequences. An exemplary database is an iCAN database. In some instances, the database comprises naïve and memory B-cell receptor sequences. In some instances, the naïve and memory B-cell receptor sequences are human, mouse, or primate sequences. In some instances, the naïve and memory B-cell receptor sequences are human sequences. In some instances, the database is analyzed for position specific variation. In some instances, antibodies described herein comprise position specific variations in CDR regions. In some instances, the CDR regions comprise multiple sites for variation.

Described herein are libraries comprising variation in a CDR region. In some instances, the CDR is CDR1, CDR2, or CDR3 of a variable heavy chain. In some instances, the CDR is CDR1, CDR2, or CDR3 of a variable light chain. In some instances, the libraries comprise multiple variants encoding for CDR1, CDR2, or CDR3. In some instances, the libraries as described herein encode for at least 50, 100, 200, 300, 400, 500, 1000, 1200, 1500, 1700, 2000, 2500, 3000, 3500, 4000, 4500, 5000, or more than 5000 CDR1 sequences. In some instances, the libraries as described herein encode for at least 50, 100, 200, 300, 400, 500, 1000, 1200, 1500, 1700, 2000, 2500, 3000, 3500, 4000, 4500, 5000, or more than 5000 CDR2 sequences. In some instances, the libraries as described herein encode for at least 50, 100, 200, 300, 400, 500, 1000, 1200, 1500, 1700, 2000, 2500, 3000, 3500, 4000, 4500, 5000, or more than 5000 CDR3 sequences. In-silico antibodies libraries are in some instances synthesized, assembled, and enriched for desired sequences.

Following synthesis of CDR1 variants, CDR2 variants, and CDR3 variants, in some instances, the CDR1 variants, the CDR2 variants, and the CDR3 variants are shuffled to generate a diverse library. In some instances, the diversity of the libraries generated by methods described herein have a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, or more than 10¹⁸ sequences. In some instances, the library has a final library diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, or more than 10¹⁸ sequences.

The germline sequences corresponding to a variant sequence may also be modified to generate sequences in a library. For example, sequences generated by methods described herein comprise at least 1, 2, 3, 4, 5, 6, 7, 8, 9, 10, 11, 12, 13, 14, 15, 16, or more than 16 mutations or variants from the germline sequence. In some instances, sequences generated comprise no more than 1, 2, 3, 4, 5, 6, 7, 8, 9, 10, 11, 12, 13, 14, 15, 16, or no more than 18 mutations or variants from the germline sequence. In some instances, sequences generated comprise about 1, 2, 3, 4, 5, 6, 7, 8, 9, 10, 11, 12, 13, 14, 15, 16, or about 18 mutations or variants relative to the germline sequence.

Coronavirus Antibody Libraries

Provided herein are libraries generated from antibody optimization methods described herein. Antibodies described herein result in improved functional activity, structural stability, expression, specificity, or a combination thereof:

Provided herein are methods and compositions relating to SARS-CoV-2 binding libraries comprising nucleic acids encoding for a SARS-CoV-2 antibody. Further provided herein are methods and compositions relating to ACE2 binding libraries comprising nucleic acids encoding for an ACE2 antibody. Such methods and compositions in some instances are generated by the antibody optimization methods and systems described herein. Libraries as described herein may be further variegated to provide for variant libraries comprising nucleic acids each encoding for a predetermined variant of at least one predetermined reference nucleic acid sequence. Further described herein are protein libraries that may be generated when the nucleic acid libraries are translated. In some instances, nucleic acid libraries as described herein are transferred into cells to generate a cell library. Also provided herein are downstream applications for the libraries synthesized using methods described herein. Downstream applications include identification of variant nucleic acids or protein sequences with enhanced biologically relevant functions, e.g., improved stability, affinity, binding, functional activity, and for the treatment or prevention of an infection caused by a coronavirus such as SARS-CoV-2.

In some instances, an antibody or antibody fragment described herein comprises a sequence of any one of SEQ ID NOs: 1-712. In some instances, an antibody or antibody fragment described herein comprises a sequence that is at least 80% identical to a sequence of any one of SEQ ID NOs: 1-712. In some instances, an antibody or antibody fragment described herein comprises a sequence that is at least 85% identical to a sequence of any one of SEQ ID NOs: 1-712. In some instances, an antibody or antibody fragment described herein comprises a sequence that is at least 90% identical to a sequence of any one of SEQ ID NOs: 1-712. In some instances, an antibody or antibody fragment described herein comprises a sequence that is at least 95% identical to a sequence of any one of SEQ ID NOs: 1-712.

In some instances, an antibody or antibody fragment described herein comprises a CDRH1 sequence of any one of SEQ ID NOs: 1-89. In some instances, an antibody or antibody fragment described herein comprises a sequence that is at least 80% identical to a CDRH1 sequence of any one of SEQ ID NOs: 1-89. In some instances, an antibody or antibody fragment described herein comprises a sequence that is at least 85% identical to a CDRH1 sequence of any one of SEQ ID NOs: 1-89. In some instances, an antibody or antibody fragment described herein comprises a sequence that is at least 90% identical to a CDRH1 sequence of any one of SEQ ID NOs: 1-89. In some instances, an antibody or antibody fragment described herein comprises a sequence that is at least 95% identical to a CDRH1 sequence of any one of SEQ ID NOs: 1-89. In some instances, an antibody or antibody fragment described herein comprises a CDRH2 sequence of any one of SEQ ID NOs: 90-178. In some instances, an antibody or antibody fragment described herein comprises a sequence that is at least 80% identical to a CDRH2 sequence of any one of SEQ ID NOs: 90-178. In some instances, an antibody or antibody fragment described herein comprises a sequence that is at least 85% identical to a CDRH2 sequence of any one of SEQ ID NOs: 90-178. In some instances, an antibody or antibody fragment described herein comprises a sequence that is at least 90% identical to a CDRH2 sequence of any one of SEQ ID NOs: 90-178. In some instances, an antibody or antibody fragment described herein comprises a sequence that is at least 95% identical to a CDRH2 sequence of any one of SEQ ID NOs: 90-178. In some instances, an antibody or antibody fragment described herein comprises a CDRH3 sequence of any one of SEQ ID NOs: 179-267. In some instances, an antibody or antibody fragment described herein comprises a sequence that is at least 80% identical to a CDRH3 sequence of any one of SEQ ID NOs: 179-267. In some instances, an antibody or antibody fragment described herein comprises a sequence that is at least 85% identical to a CDRH3 sequence of any one of SEQ ID NOs: 179-267. In some instances, an antibody or antibody fragment described herein comprises a sequence that is at least 90% identical to a CDRH3 sequence of any one of SEQ ID NOs: 179-267. In some instances, an antibody or antibody fragment described herein comprises a sequence that is at least 95% identical to a CDRH3 sequence of any one of SEQ ID NOs: 179-267.

In some instances, an antibody or antibody fragment described herein comprises a CDRL1 sequence of any one of SEQ ID NOs: 268-356. In some instances, an antibody or antibody fragment described herein comprises a sequence that is at least 80% identical to a CDRL1 sequence of any one of SEQ ID NOs: 268-356. In some instances, an antibody or antibody fragment described herein comprises a sequence that is at least 85% identical to a CDRL1 sequence of any one of SEQ ID NOs: 268-356. In some instances, an antibody or antibody fragment described herein comprises a sequence that is at least 90% identical to a CDRL1 sequence of any one of SEQ ID NOs: 268-356. In some instances, an antibody or antibody fragment described herein comprises a sequence that is at least 95% identical to a CDRL1 sequence of any one of SEQ ID NOs: 268-356. In some instances, an antibody or antibody fragment described herein comprises a CDRL2 sequence of any one of SEQ ID NOs: 357445. In some instances, an antibody or antibody fragment described herein comprises a sequence that is at least 80% identical to a CDRL2 sequence of any one of SEQ ID NOs: 357-445. In some instances, an antibody or antibody fragment described herein comprises a sequence that is at least 85% identical to a CDRL2 sequence of any one of SEQ ID NOs: 357-445. In some instances, an antibody or antibody fragment described herein comprises a sequence that is at least 90% identical to a CDRL2 sequence of any one of SEQ ID NOs: 357445. In some instances, an antibody or antibody fragment described herein comprises a sequence that is at least 95% identical to a CDRL2 sequence of any one of SEQ ID NOs: 357-445. In some instances, an antibody or antibody fragment described herein comprises a CDRL3 sequence of any one of SEQ ID NOs: 446-534. In some instances, an antibody or antibody fragment described herein comprises a sequence that is at least 80% identical to a CDRL3 sequence of any one of SEQ ID NOs: 446-534. In some instances, an antibody or antibody fragment described herein comprises a sequence that is at least 85% identical to a CDRL3 sequence of any one of SEQ ID NOs: 446-534. In some instances, an antibody or antibody fragment described herein comprises a sequence that is at least 90%/o identical to a CDRL3 sequence of any one of SEQ ID NOs: 446-534. In some instances, an antibody or antibody fragment described herein comprises a sequence that is at least 95% identical to a CDRL3 sequence of any one of SEQ ID NOs: 446-534.

In some embodiments, the antibody or antibody fragment comprising a variable domain, heavy chain region (VH) and a variable domain, light chain region (VL), wherein VH comprises complementarity determining regions CDRH1, CDRH2, and CDRH3, wherein VL comprises complementarity determining regions CDRL1, CDRL2, and CDRL3, and wherein (a) an amino acid sequence of CDRH1 is as set forth in any one of SEQ ID NOs: 1-89; (b) an amino acid sequence of CDRH2 is as set forth in any one of SEQ ID NOs: 90-178; (c) an amino acid sequence of CDRH3 is as set forth in any one of SEQ ID NOs: 179-267; (d) an amino acid sequence of CDRL1 is as set forth in any one of SEQ ID NOs: 268-356; (e) an amino acid sequence of CDRL2 is as set forth in any one of SEQ ID NOs: 357-445; and (f) an amino acid sequence of CDRL3 is as set forth in any one of SEQ ID NOs: 446-534. In some embodiments, the antibody or antibody fragment comprising a variable domain, heavy chain region (VH) and a variable domain, light chain region (VL), wherein VH comprises complementarity determining regions CDRH1, CDRH2, and CDR13, wherein VL comprises complementarity determining regions CDRL1, CDRL2, and CDRL3, and wherein (a) an amino acid sequence of CDRH1 is at least or about 80%, 85%, 90%, or 95% identical to any one of SEQ ID NOs: 1-89; (b) an amino acid sequence of CDRH2 is at least or about 80%, 85%, 90%, or 95% identical to any one of SEQ ID NOs: 90-178; (c) an amino acid sequence of CDRH3 is at least or about 80%, 85%, 90%, or 95% identical to any one of SEQ ID NOs: 179-267; (d) an amino acid sequence of CDRL1 is at least or about 80%, 85%, 90%, or 95% identical to any one of SEQ ID NOs: 268-356; (e) an amino acid sequence of CDRL2 is at least or about 80° %, 85%, 90%, or 95% identical to any one of SEQ ID NOs: 357-445; and (f) an amino acid sequence of CDRL3 is at least or about 80%, 85%, 90%, or 95% identical to any one of SEQ ID NOs: 446-534.

Described herein, in some embodiments, are antibodies or antibody fragments comprising a variable domain, heavy chain region (VH) and a variable domain, light chain region (VL), wherein the VH comprises an amino acid sequence at least about 90% identical to a sequence as set forth in any one of SEQ ID NOs: 535-623, and wherein the VL comprises an amino acid sequence at least about 90% identical to a sequence as set forth in any one of SEQ ID NOs: 624-712. In some instances, the antibodies or antibody fragments comprise VH comprising at least or about 70%, 80%, 85%, 90%, 91%, 92%, 93%, 94%, 95%, 96%, 97%, 98%, 99%, or 100% sequence identity to any one of SEQ ID NOs: 535-623, and VL comprising at least or about 70%, 80%, 85%, 90%, 910% 92%, 93%, 94%, 95%, 96%, 97%, 98%, 99%, or 100% sequence identity to any one of SEQ ID NOs: 624-712.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

Provided herein are libraries comprising nucleic acids encoding for SARS-CoV-2 antibodies. Antibodies described herein allow for improved stability for a range of SARS-CoV-2 or ACE2 binding domain encoding sequences. In some instances, the binding domain encoding sequences are determined by interactions between SARS-CoV-2 and ACE2.

Sequences of binding domains based on surface interactions between SARS-CoV-2 and ACE2 are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucse.edu/), UniProt (www.uniprot.org/), and IUPHAR/BPS Guide to PHARMACOLOGY (guidetopharmacology.org/).

Described herein are SARS-CoV-2 or ACE2 binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human. In some instances, homologous sequences are identified in the same organism, across individuals.

Following identification of SARS-CoV-2 or ACE2 binding domains, libraries comprising nucleic acids encoding for the SARS-CoV-2 or ACE2 binding domains may be generated. In some instances, libraries of SARS-CoV-2 or ACE2 binding domains comprise sequences of SARS-CoV-2 or ACE2 binding domains designed based on conformational ligand interactions, peptide ligand interactions, small molecule ligand interactions, extracellular domains of SARS-CoV-2 or ACE2, or antibodies that target SARS-CoV-2 or ACE2. Libraries of SARS-CoV-2 or ACE2 binding domains may be translated to generate protein libraries. In some instances, libraries of SARS-CoV-2 or ACE2 binding domains are translated to generate peptide libraries, immunoglobulin libraries, derivatives thereof, or combinations thereof. In some instances, libraries of SARS-CoV-2 or ACE2 binding domains are translated to generate protein libraries that are further modified to generate peptidomimetic libraries. In some instances, libraries of SARS-CoV-2 or ACE2 binding domains are translated to generate protein libraries that are used to generate small molecules.

Methods described herein provide for synthesis of libraries of SARS-CoV-2 or ACE2 binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of SARS-CoV-2 or ACE2 binding domains comprise varied nucleic acids collectively encoding variations at multiple positions. In some instances, the variant library comprises sequences encoding for variation of at least a single codon in a SARS-CoV-2 or ACE2 binding domain. In some instances, the variant library comprises sequences encoding for variation of multiple codons in a SARS-CoV-2 or ACE2 binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the SARS-CoV-2 or ACE2 binding domains, wherein the libraries comprise sequences encoding for variation of length of the SARS-CoV-2 or ACE2 binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Following identification of SARS-CoV-2 or ACE2 binding domains, antibodies may be designed and synthesized to comprise the SARS-CoV-2 or ACE2 binding domains. Antibodies comprising SARS-CoV-2 or ACE2 binding domains may be designed based on binding, specificity, stability, expression, folding, or downstream activity. In some instances, the antibodies comprising SARS-CoV-2 or ACE2 binding domains enable contact with the SARS-CoV-2 or ACE2. In some instances, the antibodies comprising SARS-CoV-2 or ACE2 binding domains enables high affinity binding with the SARS-CoV-2 or ACE2. Exemplary amino acid sequences of SARS-CoV-2 or ACE2 binding domains comprise any one of SEQ ID NOs: 1-2668.

In some instances, the SARS-CoV-2 antibody comprises a binding affinity (e.g., Kr)) to SARS-CoV-2 of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the SARS-CoV-2 antibody comprises a K_{D} of less than 1 nM. In some instances, the SARS-CoV-2 antibody comprises a K_{D} of less than 1.2 nM. In some instances, the SARS-CoV-2 antibody comprises a K_{D} of less than 2 nM. In some instances, the SARS-CoV-2 antibody comprises a Kr of less than 5 nM. In some instances, the SARS-CoV-2 antibody comprises a K_{D} of less than 10 nM. In some instances, the SARS-CoV-2 antibody comprises a K_{D} of less than 13.5 nM. In some instances, the SARS-CoV-2 antibody comprises a K_{D} of less than 15 nM. In some instances, the SARS-CoV-2 antibody comprises a K_{D} of less than 20 nM. In some instances, the SARS-CoV-2 antibody comprises a K_{D} of less than 25 nM, in some instances, the SARS-CoV-2 antibody comprises a K_{D} of less than 30 nM.

In some instances, the ACE2 antibody comprises a binding affinity (e.g., K_{D}) to ACE2 of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the ACE2 antibody comprises a K_{D} of less than 1 nM. In some instances, the ACE2 antibody comprises a K_{D} of less than 1.2 nM. In some instances, the ACE2 antibody comprises a K_{D} of less than 2 nM. In some instances, the ACE2 antibody comprises a K_{D} of less than 5 nM. In some instances, the ACE2 antibody comprises a K_{D} of less than 10 nM. In some instances, the ACE2 antibody comprises a K_{D} of less than 13.5 nM. In some instances, the ACE2 antibody comprises a K_{D} of less than 15 nM. In some instances, the ACE2 antibody comprises a K_{D} of less than 20 nM. In some instances, the ACE2 antibody comprises a K_{D} of less than 25 nM. In some instances, the ACE2 antibody comprises a K_{D} of less than 30 nM.

In some instances, the SARS-CoV-2 or ACE2 immunoglobulin is an agonist. In some instances, the SARS-CoV-2 or ACE2 immunoglobulin is an antagonist. In some instances, the SARS-CoV-2 or ACE2 immunoglobulin is an allosteric modulator. In some instances, the allosteric modulator is a negative allosteric modulator. In some instances, the allosteric modulator is a positive allosteric modulator. In some instances, the SARS-CoV-2 or ACE2 immunoglobulin results in agonistic, antagonistic, or allosteric effects at a concentration of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, 120 nM, 140 nM, 160 nM, 180 nM, 200 nM, 300 nM, 400 nM, 500 nM, 600 nM, 700 nM, 800 nM, 900 nM, 1000 nM, or more than 1000 nM. In some instances, the SARS-CoV-2 or ACE2 immunoglobulin is a negative allosteric modulator. In some instances, the SARS-CoV-2 or ACE2 immunoglobulin is a negative allosteric modulator at a concentration of at least or about 0.001, 0.005, 0.01, 0.05, 0.1, 0.5, 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM. In some instances, the SARS-CoV-2 or ACE2 immunoglobulin is a negative allosteric modulator at a concentration in a range of about 0.001 to about 100, 0.01 to about 90, about 0.1 to about 80, 1 to about 50, about 10 to about 40 nM, or about 1 to about 10 nM. In some instances, the SARS-CoV-2 or ACE2 immunoglobulin comprises an EC50 or IC50 of at least or about 0.001, 0.0025, 0.005, 0.01, 0.025, 0.05, 0.06, 0.07, 0.08, 0.9, 0.1, 0.5, 1, 2, 3, 4, 5, 6, or more than 6 nM. In some instances, the SARS-CoV-2 or ACE2 immunoglobulin comprises an EC50 or IC50 of at least or about 1 nM, 2 nM, 4 nM, 6 nM, 8 nM, 10 nM, 20 nM, 30 nM, 40 nM, 50 nM, 60 nM, 70 nM, 80 nM, 90 nM, 100 nM, or more than 100 nM.

In some instances, the affinity of the SARS-CoV-2 or ACE2 antibody generated by methods as described herein is at least or about 1.5×, 2.0×, 5×, 10×, 20×, 30×, 40×, 50×, 60×, 70×, 80×, 90×, 100×, 200×, or more than 200× improved binding affinity as compared to a comparator antibody. In some instances, the SARS-CoV-2 or ACE2 antibody generated by methods as described herein is at least or about 1.5×, 2.0×, 5×, 10×, 20×, 30×, 40×, 50×, 60×, 70×, 80×, 90×, 100×, 200×, or more than 200× improved function as compared to a comparator antibody. In some instances, the comparator antibody is an antibody with similar structure, sequence, or antigen target.

Provided herein are SARS-CoV-2 or ACE2 binding libraries comprising nucleic acids encoding for antibodies comprising SARS-CoV-2 or ACE2 binding domains comprise variation in domain type, domain length, or residue variation. In some instances, the domain is a region in the antibody comprising the SARS-CoV-2 or ACE2 binding domains. For example, the region is the VH, CDRH3, or VL domain. In some instances, the domain is the SARS-CoV-2 or ACE2 binding domain.

Methods described herein provide for synthesis of a SARS-CoV-2 or ACE21 binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the SARS-CoV-2 or ACE2 binding library comprises varied nucleic acids collectively encoding variations at multiple positions. In some instances, the variant library comprises sequences encoding for variation of at least a single codon of a VH or VL domain. In some instances, the variant library comprises sequences encoding for variation of at least a single codon in a SARS-CoV-2 or ACE2 binding domain. For example, at least one single codon of a SARS-CoV-2 or ACE2 binding domain is varied. In some instances, the variant library comprises sequences encoding for variation of multiple codons of a VH or VL domain. In some instances, the variant library comprises sequences encoding for variation of multiple codons in a SARS-CoV-2 or ACE2 binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a SARS-CoV-2 or ACE2 binding library of nucleic acids each encoding for a predetermined variant of at least one predetermined reference nucleic acid sequence, wherein the SARS-CoV-2 or ACE2 binding library comprises sequences encoding for variation of length of a domain. In some instances, the domain is VH or VL domain. In some instances, the domain is the SARS-CoV-2 or ACE2binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are SARS-CoV-2 or ACE2 binding libraries comprising nucleic acids encoding for antibodies comprising SARS-CoV-2 or ACE2 binding domains, wherein the SARS-CoV-2 or ACE2 binding libraries are synthesized with various numbers of fragments. In some instances, the fragments comprise the VH or VL domain. In some instances, the SARS-CoV-2 or ACE2 binding libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

SARS-CoV-2 or ACE2 binding libraries comprising nucleic acids encoding for antibodies comprising SARS-CoV-2 or ACE2 binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145.25 to 140, 30 to 135, 35 to 130.40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

SARS-CoV-2 or ACE2 binding libraries comprising de novo synthesized variant sequences encoding for antibodies comprising SARS-CoV-2 or ACE2 binding domains comprise a number of variant sequences. In some instances, a number of variant sequences is de novo synthesized for a CDRH1, CDRH2, CDRH3, CDRL1, CDRL2, CDRL3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences are de novo synthesized for a SARS-CoV-2 or ACE2 binding domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

SARS-CoV-2 or ACE2 binding libraries comprising de novo synthesized variant sequences encoding for antibodies comprising SARS-CoV-2 or ACE2 binding domains comprise improved diversity. In some instances, variants include affinity maturation variants. Alternatively or in combination, variants include variants in other regions of the antibody including, but not limited to, CDRH1, CDRH2, CDRL1, CDRL2, and CDRL3. In some instances, the number of variants of the SARS-CoV-2 or ACE2 binding libraries is least or about 10⁴, 10⁵, 10⁶, 10⁷, 10⁸, 10⁹, 10¹⁰, 10¹¹, 10¹², 10¹³, 10¹⁴ or more than 10¹⁴ non-identical sequences.

Following synthesis of SARS-CoV-2 or ACE2 binding libraries comprising nucleic acids encoding antibodies comprising SARS-CoV-2 or ACE2 binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. For example, SARS-CoV-2 or ACE2 binding libraries comprise nucleic acids encoding antibodies comprising SARS-CoV-2 or ACE2 binding domains with multiple tags such as GFP. FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

As used herein, the term antibody will be understood to include proteins having the characteristic two-armed. Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL. VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for an antibody, wherein the antibody is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for an antibody, wherein the antibody is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules. e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

In some embodiments, the antibody is a multivalent antibody. In some embodiments, the antibody is a monovalent, bivalent, or multivalent antibody. In some instances, the antibody is monospecific, bispecific, or multispecific. In some embodiments, the antibody is monovalent monospecific, monovalent bispecific, monovalent multispecific, bivalent monospecific, bivalent bispecific, bivalent multispecific, multivalent monospecific, multivalent bispecific, multivalent multispecific. In some instances, the antibody is homodimeric, heterodimeric, or heterotrimeric.

In some embodiments, libraries comprise immunoglobulins that are adapted to the species of an intended therapeutic target. Generally, these methods include “mammalization” and comprises methods for transferring donor antigen-binding information to a less immunogenic mammal antibody acceptor to generate useful therapeutic treatments. In some instances, the mammal is mouse, rat, equine, sheep, cow, primate (e.g., chimpanzee, baboon, gorilla, orangutan, monkey), dog, cat, pig, donkey, rabbit, and human. In some instances, provided herein are libraries and methods for felinization and caninization of antibodies.

“Humanized” forms of non-human antibodies can be chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance.

“Caninization” can comprise a method for transferring non-canine antigen-binding information from a donor antibody to a less immunogenic canine antibody acceptor to generate treatments useful as therapeutics in dogs. In some instances, caninized forms of non-canine antibodies provided herein are chimeric antibodies that contain minimal sequence derived from non-canine antibodies. In some instances, caninized antibodies are canine antibody sequences (“acceptor” or “recipient” antibody) in which hypervariable region residues of the recipient are replaced by hypervariable region residues from a non-canine species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the canine antibody are replaced by corresponding non-canine FR residues. In some instances, caninized antibodies include residues that are not found in the recipient antibody or in the donor antibody. In some instances, these modifications are made to further refine antibody performance. The caninized antibody may also comprise at least a portion of an immunoglobulin constant region (Fc) of a canine antibody.

“Felinization” can comprise a method for transferring non-feline antigen-binding information from a donor antibody to a less immunogenic feline antibody acceptor to generate treatments useful as therapeutics in cats. In some instances, felinized forms of non-feline antibodies provided herein are chimeric antibodies that contain minimal sequence derived from non-feline antibodies. In some instances, felinized antibodies are feline antibody sequences (“acceptor” or “recipient” antibody) in which hypervariable region residues of the recipient are replaced by hypervariable region residues from a non-feline species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the feline antibody are replaced by corresponding non-feline FR residues. In some instances, felinized antibodies include residues that are not found in the recipient antibody or in the donor antibody. In some instances, these modifications are made to further refine antibody performance. The felinized antibody may also comprise at least a portion of an immunoglobulin constant region (Fc) of a felinize antibody.

Methods as described herein may be used for optimization of libraries encoding a non-immunoglobulin. In some instances, the libraries comprise antibody mimetics. Exemplary antibody mimetics include, but are not limited to, anticalins, affilins, affibody molecules, affimers, affitins, alphabodies, avimers, atrimers, DARPins, fynomers, Kunitz domain-based proteins, monobodies, anticalins, knottins, armadillo repeat protein-based proteins, and bicyclic peptides.

Libraries described herein comprising nucleic acids encoding for an antibody comprise variations in at least one region of the antibody. Exemplary regions of the antibody for variation include, but are not limited to, a complementarity-determining region (CDR), a variable domain, or a constant domain. In some instances, the CDR is CDR1, CDR2, or CDR3. In some instances, the CDR is a heavy domain including, but not limited to, CDRH1, CDRH2, and CDRH3. In some instances, the CDR is a light domain including, but not limited to, CDRL1, CDRL2, and CDRL3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH).

Methods described herein provide for synthesis of libraries comprising nucleic acids encoding an antibody, wherein each nucleic acid encodes for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the antibody library comprises varied nucleic acids collectively encoding variations at multiple positions. In some instances, the variant library comprises sequences encoding for variation of at least a single codon of a CDRH1, CDRH2, CDRH3, CDRL1, CDRL2, CDRL3, VL, or VH domain. In some instances, the variant library comprises sequences encoding for variation of multiple codons of a CDRH1, CDRH2, CDRH3, CDRL1, CDRL2, CDRL3, VL, or VH domain. In some instances, the variant library comprises sequences encoding for variation of multiple codons of framework element 1 (FW1), framework element 2 (FW2), framework element 3 (FW3), or framework element 4 (FW4). An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 10, 175, 225, 250, 275, 300, or more than 300 codons.

In some instances, the at least one region of the antibody for variation is from heavy chain V-gene family, heavy chain D-gene family, heavy chain J-gene family, light chain V-gene family, or light chain J-gene family. In some instances, the light chain V-gene family comprises immunoglobulin kappa (IGK) gene or immunoglobulin lambda (IGL). Exemplary regions of the antibody for variation include, but are not limited to, IGHV1-18, IGHV1-69, IGHV1-8, IGHV3-21, IGHV3-23, IGHV3-30/33rn, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGHV1-69, IGHV3-30, IGHV3-23, IGHV3, IGIV1-46, IGHV3-7, IGHV1, or IGHV1-8. In some instances, the gene is IGHV1-69 and IGHV3-30. In some instances, the region of the antibody for variation is IGHJ3, IGHJ6, IGHJ, IGIJ4, IGIJ5, IGIJ2, or IGH1. In some instances, the region of the antibody for variation is IGHJ3, IGHJ6, IGHJ, or IGHJ4. In some instances, the at least one region of the antibody for variation is IGHV1-69, IGHV3-23, IGKV3-20, TGKV1-39, or combinations thereof. In some instances, the at least one region of the antibody for variation is IGHV1-69 and IGKV3-20, In some instances, the at least one region of the antibody for variation is IGHV1-69 and IGKV1-39. In some instances, the at least one region of the antibody for variation is IGHV3-23 and IGKV3-20. In some instances, the at least one region of the antibody for variation is IGHV3-23 and IGKV1-39.

Provided herein are libraries comprising nucleic acids encoding for antibodies, wherein the libraries are synthesized with various numbers of fragments. In some instances, the fragments comprise the CDRH1, CDRH2, CDRH3, CDRL1, CDRL2, CDRL3, VL, or VH domain. In some instances, the fragments comprise framework element 1 (FW1), framework element 2 (FW2), framework element 3 (FW3), or framework element 4 (FW4). In some instances, the antibody libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400.275 to 375, or 300 to 350 base pairs.

Libraries comprising nucleic acids encoding for antibodies as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

A number of variant sequences for the at least one region of the antibody for variation are de novo synthesized using methods as described herein. In some instances, a number of variant sequences is de novo synthesized for CDRH1, CDRL12, CDRH3, CDRL1, CDRL2, CDRL3, VL, VH, or combinations thereof. In some instances, a number of variant sequences is de novo synthesized for framework element 1 (FW1), framework element 2 (FW2), framework element 3 (FW3), or framework element 4 (FW4). The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is at least or about 500, 600, 700. 800, 900, 1000, 2000, 3000, 4000, 5000, 6000, 7000, 8000, or more than 8000 sequences. In some instances, the number of variant sequences is about 10 to 500, 25 to 475, 50 to 450, 75 to 425, 100 to 400, 125 to 375, 150 to 350, 175 to 325, 200 to 300, 225 to 375, 250 to 350, or 275 to 325 sequences.

Variant sequences for the at least one region of the antibody, in some instances, vary in length or sequence. In some instances, the at least one region that is de novo synthesized is for CDRH1, CDRH2, CDRH3, CDRL1, CDRL2, CDRL3, VL, VH, or combinations thereof. In some instances, the at least one region that is de novo synthesized is for framework element 1 (FW1), framework element 2 (FW2), framework element 3 (FW3), or framework element 4 (FW4). In some instances, the variant sequence comprises at least or about 1, 2, 3, 4, 5, 6, 7, 8, 9, 10, 15, 20, 25, 30, 35, 40, 45, 50, or more than 50 variant nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 additional nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 less nucleotides or amino acids as compared to wild-type. In some instances, the libraries comprise at least or about 10¹, 10², 10³, 10⁴, 10⁵, 10⁶, 10⁷, 10⁸, 10⁹, 10¹⁰ or more than 10¹⁰ variants.

Following synthesis of antibody libraries, antibody libraries may be used for screening and analysis. For example, antibody libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, antibody libraries are assayed by sequencing using various methods including, but not limited to, single-molecule real-time (SMRT) sequencing. Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. In some instances, antibody libraries are displayed on the surface of a cell or phage. In some instances, antibody libraries are enriched for sequences with a desired activity using phage display.

In some instances, the antibody libraries are assayed for functional activity, structural stability (e.g., thermal stable or pH stable), expression, specificity, or a combination thereof. In some instances, the antibody libraries are assayed for antibody capable of folding. In some instances, a region of the antibody is assayed for functional activity, structural stability, expression, specificity, folding, or a combination thereof. For example, a VH region or VL, region is assayed for functional activity, structural stability, expression, specificity, folding, or a combination thereof.

In some instances, the affinity of antibodies or IgGs generated by methods as described herein is at least or about 1.5×, 2.0×, 5×, 10×, 20×, 30×, 40×, 50×, 60×, 70×, 80×, 90×, 100×, 200×, or more than 200× improved binding affinity as compared to a comparator antibody. In some instances, the affinity of antibodies or IgGs generated by methods as described herein is at least or about 1.5×, 2.0×, 5×, 10×, 20×, 30×, 40×, 50×, 60×, 70×, 80×, 90×, 100×, 200×, or more than 200× improved function as compared to a comparator antibody. In some instances, the comparator antibody is an antibody with similar structure, sequence, or antigen target.

Expression Systems

Provided herein are libraries comprising nucleic acids encoding for antibody comprising binding domains, wherein the libraries have improved specificity, stability, expression, folding, or downstream activity. In some instances, libraries described herein are used for screening and analysis.

Provided herein are libraries comprising nucleic acids encoding for antibody comprising binding domains, wherein the nucleic acid libraries are used for screening and analysis. In some instances, screening and analysis comprises in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, rabbit, primate, and insect.

Nucleic acid libraries described herein may be screened for various pharmacological or pharmacokinetic properties. In some instances, the libraries are screened using in vitro assays, in vivo assays, or in vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Provided herein are nucleic acid libraries, wherein the nucleic acid libraries may be expressed in a vector. Expression vectors for inserting nucleic acid libraries disclosed herein may comprise eukaryotic or prokaryotic expression vectors. Exemplary expression vectors include, without limitation, mammalian expression vectors: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 Vector, pEF1a-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/VS-His A and pDEST5. In some instances, the vector is pcDNA3 or pcDNA3.1.

Described herein are nucleic acid libraries that are expressed in a vector to generate a construct comprising an antibody. In some instances, a size of the construct varies. In some instances, the construct comprises at least or about 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for antibodies, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy. Fluorescence Activated Cell Sorting (FACS), fluorescence microscopy), and antibiotic resistance determination.

Diseases and Disorders

Provided herein are SARS-CoV-2 or ACE2 binding libraries comprising nucleic acids encoding for antibodies comprising SARS-CoV-2 or ACE2 binding domains may have therapeutic effects. In some instances, the SARS-CoV-2 or ACE2 binding libraries result in protein when translated that is used to treat a disease or disorder. In some instances, the protein is an immunoglobulin. In some instances, the protein is a peptidomimetic. In some instances, the disease or disorder is a viral infection caused by SARS-CoV-2. In some instances, the disease or disorder is a respiratory disease or disorder caused by SARS-CoV-2.

SARS-CoV-2 or ACE2 variant antibody libraries as described herein may be used to treat SARS-CoV-2. In some embodiments, the SARS-CoV-2 or ACE2 variant antibody libraries are used to treat or prevent symptoms of SARS-CoV-2. These symptoms include, but are not limited to, fever, chills, cough, fatigue, headaches, loss of taste, loss of smell, nausea, vomiting, muscle weakness, sleep difficulties, anxiety, and depression. In some embodiments, the SARS-CoV-2 or ACE2 variant antibody libraries are used to treat a subject who has symptoms for an extended period of time. In some embodiments, the subject has symptoms for an extended period of time after testing negative for SARS-CoV-2. In some embodiments, the subject has symptoms for an extended period of time including at least 1 week, 2 weeks, 3 weeks, 1 month, 2 months, 3 months, 4 months, 5 months, 6 months, 7 months, 8 months, 9 months, 10 months, 11 months, 1 year, or more than 1 year.

In some instances, the subject is a mammal. In some instances, the subject is a mouse, rabbit, dog, or human. Subjects treated by methods described herein may be infants, adults, or children. Pharmaceutical compositions comprising antibodies or antibody fragments as described herein may be administered intravenously or subcutaneously. In some instances, a pharmaceutical composition comprises an antibody or antibody fragment described herein comprising a CDRH1 sequence of any one of SEQ ID NOs: 1-50, 779-919, 1344-1523, and 2381-2452. In some instances, a pharmaceutical composition comprises an antibody or antibody fragment described herein comprising a CDRH2 sequence of any one of SEQ ID NOs: 51-100, 920-1061, 1524-1703, and 2453-2524 In some instances, a pharmaceutical composition comprises an antibody or antibody fragment described herein comprising a CDRH3 sequence of any one of SEQ ID NOs: 101-150, 1062-1202, 1704-1883, and 2525-2596. In some instances, a pharmaceutical composition comprises an antibody or antibody fragment described herein comprising a variable domain, heavy chain region (VH) and a variable domain, light chain region (VL), wherein VH comprises complementarity determining regions CDRH1, CDRH2, and CDRH3, wherein VL comprises complementarity determining regions CDRL1, CDRL2, and CDRL3, and wherein (a) an amino acid sequence of CDRH1 is as set forth in any one of SEQ ID NOs: 1-89; (b) an amino acid sequence of CDRH2 is as set forth in any one of SEQ ID NOs: 90-178; (c) an amino acid sequence of CDRH3 is as set forth in any one of SEQ ID NOs: 179-267; (d) an amino acid sequence of CDRL1 is as set forth in any one of SEQ ID NOs: 268-356; (e) an amino acid sequence of CDRL2 is as set forth in any one of SEQ ID NOs: 357-445; and (f) an amino acid sequence of CDRL3 is as set forth in any one of SEQ ID NOs: 446-534. In some instances, a pharmaceutical composition comprises an antibody or antibody fragment described herein comprising a VH comprising at least or about 70%, 80%, 85%, 90%, 91%.92%, 93%, 94%, 95%, 96%, 97%, 98%, 99%, or 100% sequence identity to any one of SEQ ID NOs: 535-623, and VL comprising at least or about 70%, 80%, 85%, 90%, 91%, 92%, 93%, 94%, 95%, 96%, 97%, 98%, 99%, or 100% sequence identity to any one of SEQ ID NOs: 624-712. In some instances, a pharmaceutical composition comprises an antibody or antibody fragment described herein comprising a heavy chain variable domain comprising at least or about 70%, 80%, 85%, 90%, 91%, 92%, 93%, 94%, 95%, 96%, 97%, 98%, 99%, or 100% sequence identity to any one of SEQ ID NOs: 1918-2058, 2599-2778, and 3095-3173.

SARS-CoV-2 or ACE2 antibodies as described herein may confer immunity after exposure to SARS-CoV-2 or ACE2 antibodies. In some embodiments, the SARS-CoV-2 or ACE2 antibodies described herein are used for passive immunization of a subject. In some instances, the subject is actively immunized ater exposure to SARS-CoV-2 or ACE2 antibodies followed by exposure to SARS-CoV-2. In some embodiments. SARS-CoV-2 or ACE2 antibodies are derived from a subject who has recovered from SARS-CoV-2.

In some embodiments, the immunity occurs at least about 30 minutes, 1 hour, 5 hours, 10 hours, 16 hours, 20 hours, 24 hours, 2 days, 3 days, 4 days, 5 days, 6 days, 1 week, 2 weeks, or more than 2 weeks after exposure to SARS-CoV-2 or ACE2 antibodies. In some instances, the immunity lasts for at least about 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more than 5 years after exposure to SARS-CoV-2 or ACE2 antibodies.

In some embodiments, the subject receives the SARS-CoV-2 or ACE2 antibodies prior to exposure to SARS-CoV-2. In some embodiments, the subject receives the SARS-CoV-2 or ACE2 antibodies at least about 30 minutes, 1 hour, 4 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more than 5 years prior to exposure to SARS-CoV-2. In some embodiments, the subject receives the SARS-CoV-2 or ACE2 antibodies after exposure to SARS-CoV-2. In some embodiments, the subject receives the SARS-CoV-2 or ACE2 antibodies at least about 30 minutes, 1 hour, 4 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, 5 years, or more than 5 years after exposure to SARS-CoV-2.

SARS-CoV-2 or ACE2 antibodies described herein may be administered through various routes. The administration may, depending on the composition being administered, for example, be oral, pulmonary, intravenous, intraperitoneal, intramuscular, intracavity, subcutaneous, or transdermal.

Described herein are compositions or pharmaceutical compositions comprising SARS-CoV-2 or ACE2 antibodies or antibody fragment thereof that comprise various dosages of the antibodies or antibody fragment. In some instances, the dosage is ranging from about 1 to 25 mg/kg, from about 1 to 50 mg/kg, from about 1 to 80 mg/kg, from about 1 to about 100 mg/kg, from about 5 to about 100 mg/kg, from about 5 to about 80 mg/kg, from about 5 to about 60 mg/kg, from about 5 to about 50 mg/kg or from about 5 to about 500 mg/kg which can be administered in single or multiple doses. In some instances, the dosage is administered in an amount of about 0.01 mg/kg, about 0.05 mg/kg, about 0.10 mg/kg, about 0.25 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 105 mg/kg, about 110 mg/kg, about 115 mg/kg, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 240, about 250, about 260, about 270, about 275, about 280, about 290, about 300, about 310, about 320, about 330, about 340, about 350, about 360 mg/kg, about 370 mg/kg, about 380 mg/kg, about 390 mg/kg, about 400 mg/kg, 410 mg/kg, about 420 mg/kg, about 430 mg/kg, about 440 mg/kg, about 450 mg/kg, about 460 mg/kg, about 470 mg/kg, about 480 mg/kg, about 490 mg/kg, or about 500 mg/kg.

SARS-CoV-2 or ACE2 antibodies or antibody fragment thereof described herein, in some embodiments, improve disease severity. In some embodiments, the SARS-CoV-2 or ACE2 antibodies or antibody fragment thereof improve disease severity at a dose level of about 0.01 mg/kg, about 0.05 mg/kg, about 0.10 mg/kg, about 0.25 mg/kg, about 0.5 mg/kg, about 1 mg/kg, about 5 mg/kg, about 10 mg/kg, about 15 mg/kg, or about 20 mg/kg. In some embodiments, the SARS-CoV-2 or ACE2 antibodies or antibody fragment thereof improve disease severity at a dose level of about 1 mg/kg, about 5 mg/kg, or about 10 mg/kg. In some embodiments, disease severity is determined by percent weight loss. In some embodiments, the SARS-CoV-2 or ACE2 antibodies or antibody fragment thereof protects against weight loss at a dose level of about 0.01 mg/kg, about 0.05 mg/kg, about 0.10 mg/kg, about 0.25 mg/kg, about 0.5 mg/kg, about 1 mg/kg, about 5 mg/kg, about 10 mg/kg, about 15 mg/kg, or about 20 mg/kg. In some embodiments, the SARS-CoV-2 or ACE2 antibodies or antibody fragment thereof protects against weight loss at a dose level of about 1 mg/kg, about 5 mg/kg, or about 10 mg/kg. In some embodiments, SARS-CoV-2 or ACE2 antibodies or antibody fragment thereof

Variant Libraries

Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation. Table 1 provides a listing of each codon possible (and the representative amino acid) for a variant site.

TABLE L
List of codons and amino acids

| | One | Three | |
|---|---|---|---|
| | letter | letter | |
| Amino Acids | code | code | Codons |
| Alanine | A | Ala | GCA GCC GCG GCT |
| Cysteine | C | Cys | TGC TGT |
| Aspartic acid | D | Asp | GAC GAT |
| Glutamic acid | E | Glu | GAA GAG |
| Phenylalanine | F | Phe | TTC TTT |
| Glycine | G | Gly | GGA GGC GGG GCT |
| Histidine | M | His | CAC CAT |
| isoleucine | I | Iso | ATA ATC ATT |
| Lysine | K | Lys | AAA AAG |
| Leucine | L | Leu | TTA TTG CTA CTC CTG CTT |
| Methionine | M | Met | ATG |
| Asparagine | N | Asn | AAC AAT |
| Proline | P | Pro | CCA CCC CCG CCT |
| Glutamine | Q | Gln | CAA CAG |
| Arginine | R | Arg | AGA AGG CGA CGC CGG CGT |
| Serine | S | Ser | AGG AGT TCA TCC TCG TCT |
| Threonine | T | Thr | ACA ACC ACG ACT |
| Valine | V | Val | GTA GTC GTG GTT |
| Tryptophan | W | Trp | TGG |
| Tyrosine | Y | Tyr | TAC TAT |

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform is capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene variants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations or variants at a specific site within the receptor, represents one approach to this development challenge. Though costly and time and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library, enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region. Fab region, variable region of the Fab region, constant region of the Fab region, variable domain of the heavy chain or light chain (V_{H} or V_{L}), and specific complementarity-determining regions (CDRs) of V_{H} or V_{L}.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system. LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two dimensional surface. e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 um. In some instances, the distance between the centers of two adjacent loci is less than about 200, 150, 100, 80, 70, 60, 50, 40, 30, 20 or 10 um. In some instances, each locus has a width of about 0.5, 1, 2, 3, 4, 5, 6, 7, 8, 9, 10, 20, 30, 40, 50, 60, 70, 80, 90 or 100 um. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm, 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 nm.

Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive. e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation; nylon, both modified and unmodified, nitrocellulose, polypropylene, and the like. In some instances, a substrate comprises rigid materials. For rigid materials, materials can include, without limitation: glass; fuse silica; silicon, plastics (for example polytetrafluornethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups. (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once. i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using. e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloracetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150, 22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 1, 10 nucleotides, or less. The length of each of the polynucleotides or average length of the polynucleotides within the device may fall from 10-500, 9-400, 11-300, 12-200, 13-150, 14-100, 15-50, 16-45, 1740, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

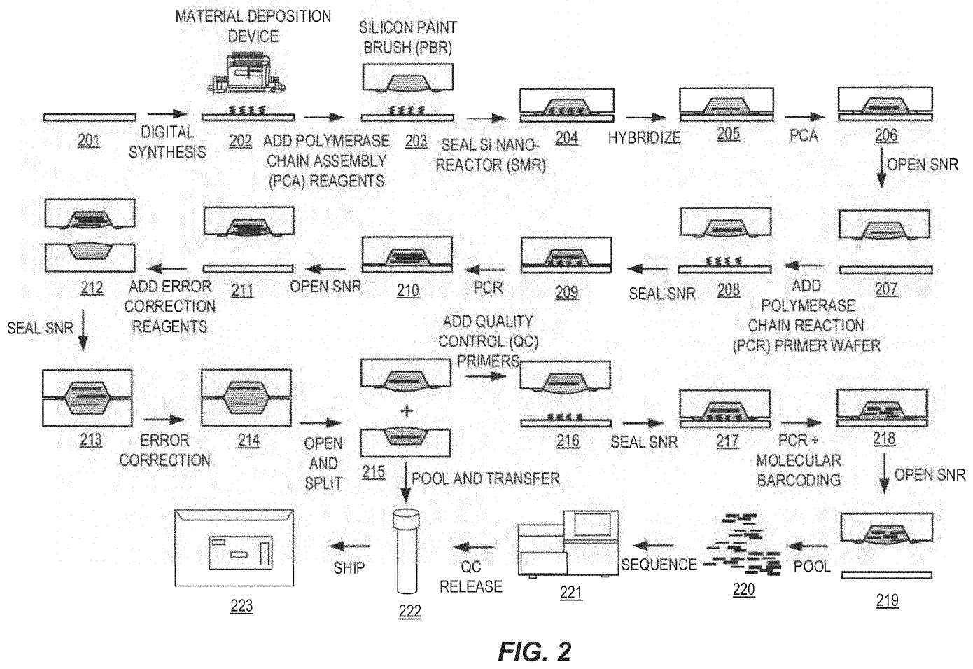

Referring to the Figures. FIG. 2 illustrates an exemplary process workflow for synthesis of nucleic acids (e.g., genes) from shorter nucleic acids. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the work-flow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device 201, is designed to release reagents in a step wise fashion such that multiple polynucleotides extend, in parallel, one residue at a time to generate oligomers with a predetermined nucleic acid sequence 202. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library 203. Prior to or after the sealing 204 of the polynucleotides, a reagent is added to release the polynucleotides from the substrate. In the exemplary workflow, the polynucleotides are released subsequent to sealing of the nanoreactor 205. Once released, fragments of single stranded polynucleotides hybridize in order to span an entire long range sequence of DNA. Partial hybridization 205 is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for forming a complete large span of double stranded DNA 206.

After PCA is complete, the nanoreactor is separated from the device 207 and positioned for interaction with a device having primers for PCR 208. After sealing, the nanoreactor is subject to PCR 209 and the larger nucleic acids are amplified. After PCR 210, the nanochamber is opened 211, error correction reagents are added 212, the chamber is sealed 213 and an error correction reaction occurs to remove mismatched base pairs and/or strands with poor complementarity from the double stranded PCR amplification products 214. The nanoreactor is opened and separated 215. Error corrected product is next subject to additional processing steps, such as PCR and molecular bar coding, and then packaged 222 for shipment 223.

In some instances, quality control measures are taken. After error correction, quality control steps include for example interaction with a wafer having sequencing primers for amplification of the error corrected product 216, sealing the wafer to a chamber containing error corrected amplification product 217, and performing an additional round of amplification 218. The nanoreactor is opened 219 and the products are pooled 220 and sequenced 221. After an acceptable quality control determination is made, the packaged product 222 is approved for shipment 223.

In some instances, a nucleic acid generate by a workflow such as that in FIG. 2 is subject to mutagenesis using overlapping primers disclosed herein. In some instances, a library of primers are generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence 202.

Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

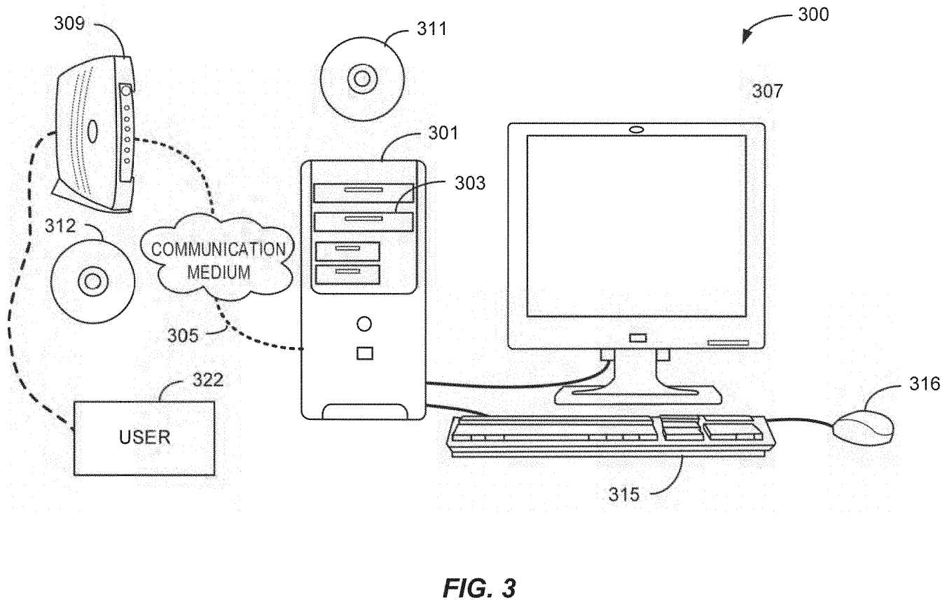

The computer system 300 illustrated in FIG. 3 may be understood as a logical apparatus that can read instructions from media 311 and/or a network port 305, which can optionally be connected to server 309 having fixed media 312. The system, such as shown in FIG. 3 can include a CPU 301, disk drives 303, optional input devices such as keyboard 315 and/or mouse 316 and optional monitor 307. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 322 as illustrated in FIG. 3.

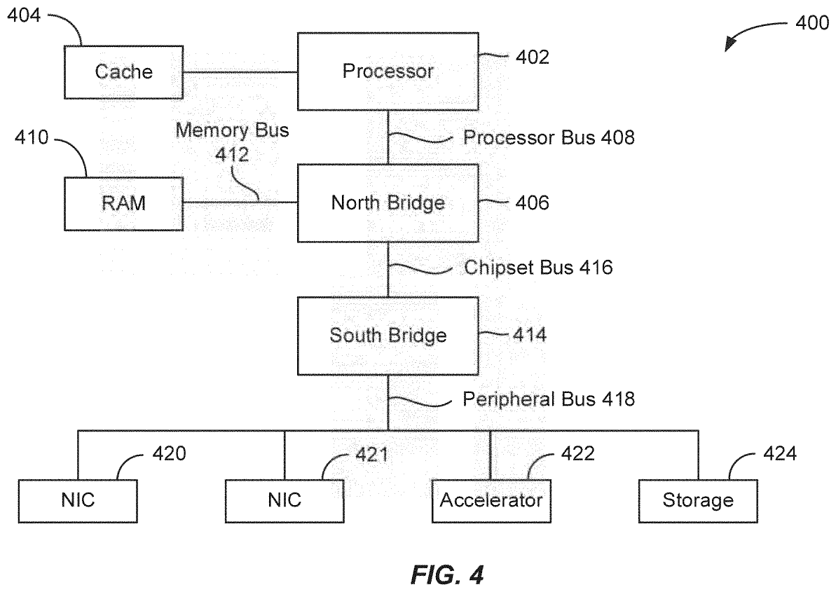

FIG. 4 is a block diagram illustrating a first example architecture of a computer system 400 that can be used in connection with example instances of the present disclosure. As depicted in FIG. 4, the example computer system can include a processor 402 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor. Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 4, a high speed cache 404 can be connected to, or incorporated in, the processor 402 to provide a high speed memory for instructions or data that have been recently, or are frequently, used by processor 402. The processor 402 is connected to a north bridge 406 by a processor bus 408. The north bridge 406 is connected to random access memory (RAM) 410 by a memory bus 412 and manages access to the RAM 410 by the processor 402. The north bridge 406 is also connected to a south bridge 414 by a chipset bus 416. The south bridge 414 is, in turn, connected to a peripheral bus 418. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 418. In some alternative architectures, the functionality of the north bridge can be incorporated into the processor instead of using a separate north bridge chip. In some instances, system 400 can include an accelerator card 422 attached to the peripheral bus 418. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 424 and can be loaded into RAM 410 and/or cache 404 for use by the processor. The system 400 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example instances of the present disclosure. In this example, system 400 also includes network interface cards (NICs) 420 and 421 connected to the peripheral bus for providing network interfaces to external storage, such as Network Attached Storage (NAS) and other computer systems that can be used for distributed parallel processing.

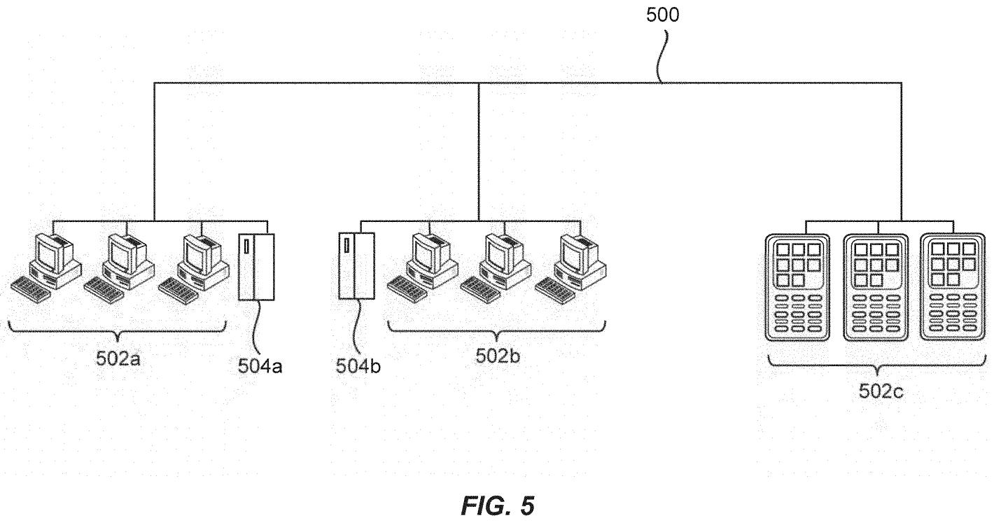

FIG. 5 is a diagram showing a network 500 with a plurality of computer systems 502a, and 502b, a plurality of cell phones and personal data assistants 502c, and Network Attached Storage (NAS) 504a, and 504b. In example instances, systems 502a, 502b, and 502c can manage data storage and optimize data access for data stored in Network Attached Storage (NAS) 504a and 504b. A mathematical model can be used for the data and be evaluated using distributed parallel processing across computer systems 502a, and 502b, and cell phone and personal data assistant systems 502c. Computer systems 502a, and 502b, and cell phone and personal data assistant systems 502c can also provide parallel processing for adaptive data restructuring of the data stored in Network Attached Storage (NAS) 504a and 504b. FIG. 5 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

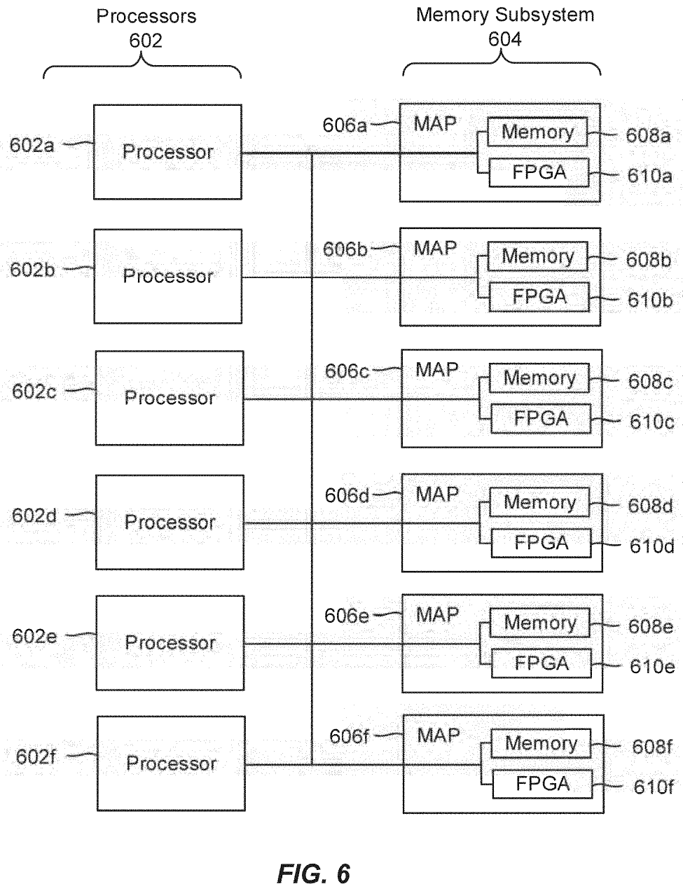

FIG. 6 is a block diagram of a multiprocessor computer system using a shared virtual address memory space in accordance with an example instance. The system includes a plurality of processors 602a-f that can access a shared memory subsystem 604. The system incorporates a plurality of programmable hardware memory algorithm processors (MAPs) 606a-f in the memory subsystem 604. Each MAP 606a-f can comprise a memory 608a-f and one or more field programmable gate arrays (FPGAs) 610a-f. The MAP provides a configurable functional unit and particular algorithms or portions of algorithms can be provided to the FPGAs 610a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 608a-f, allowing it to execute tasks independently of, and asynchronously from the respective microprocessor 602a-f, in this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors. FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 4, system on chips (SOCs), application specific integrated circuits (ASICs), or other processing and logic elements. For example, the Set Processor and Optimizer can be implemented with hardware acceleration through the use of a hardware accelerator card, such as accelerator card 422 illustrated in FIG. 4.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Functionalization of a Device Surface

A device was functionalized to support the attachment and synthesis of a library of polynucleotides. The device surface was first wet cleaned using a piranha solution comprising 90% H₂SO₄ and 10% H₂O₂ for 20 minutes. The device was rinsed in several beakers with DI water, held under a DI water gooseneck faucet for 5 min, and dried with N₂. The device was subsequently soaked in NH₄OH (1:100; 3 mL:300 mL) for 5 min, rinsed with DI water using a handgun, soaked in three successive beakers with DI water for 1 min each, and then rinsed again with DI water using the handgun. The device was then plasma cleaned by exposing the device surface to O₂. A SAMCO PC-300 instrument was used to plasma etch O₂ at 250 watts for 1 min in downstream mode.

The cleaned device surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The device surface was resist coated using a Brewer Science 200× spin coater. SPR™ 3612 photoresist was spin coated on the device at 2500 rpm for 40 sec. The device was pre-baked for 30 min at 90° C. on a Brewer hot plate. The device was subjected to photolithography using a Karl Suss MA6 mask aligner instrument. The device was exposed for 2.2 sec and developed for 1 min in MSF 26A. Remaining developer was rinsed with the handgun and the device soaked in water for 5 min. The device was baked for 30 min at 100° C. in the oven, followed by visual inspection for lithography defects using a Nikon L200. A descum process was used to remove residual resist using the SAMCO PC-300 instrument to O₂ plasma etch at 250 watts for 1 min.

The device surface was passively functionalized with a 100 μL solution of perfluorooctyltrichlorosilane mixed with 10 μL light mineral oil. The device was placed in a chamber, pumped for 10 min, and then the valve was closed to the pump and left to stand for 10 min. The chamber was vented to air. The device was resist stripped by performing two soaks for 5 min in 500 mL NMP at 70° C. with ultrasonication at maximum power (9 on Crest system). The device was then soaked for 5 min in 500 mL isopropanol at room temperature with ultrasonication at maximum power. The device was dipped in 300 mL of 200 proof ethanol and blown dry with N₂. The functionalized surface was activated to serve as a support for polynucleotide synthesis.

### Example 2: Synthesis of a 50-Mer Sequence on an Oligonucleotide Synthesis Device

A two dimensional oligonucleotide synthesis device was assembled into a flowcell, which was connected to a flowcell (Applied Biosystems (ABI394 DNA Synthesizer”). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described. 5′AGACAATCAACCATTTGGGGTGGACAGCCTTGACCTCTAGACTTCGGCAT##TTTTTTTTTT3′, (SEQ ID NO: 714) where # denotes Thymidine-succinyl hexamide CED phosphoramidite (CLP-2244 from ChemGenes), which is a cleavable linker enabling the release of oligos from the surface during deprotection.

The synthesis was done using standard DNA synthesis chemistry (coupling, capping, oxidation, and deblocking) according to the protocol in Table 2 and an ABI synthesizer.

TABLE 2

| General DN A Synthesis | | |
|---|---|---|
| Process Name | Process Step | Time (sec) |
| WASH (Acetonitrile | Acetonitrile System Flush | 4 |
| WashFlow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flow cell | 6 |
| Activator Flow) | Activator + Phosphoramidite | 6 |
| | to Flowcell | |
| | Activator to Flowcell | 0.5 |
| | Activator + Phosphoramidite | 5 |
| | to Flowcell | |
| | Activator to Flowcell | 0.5 |
| | Activator + Phosphoramidite | 5 |
| | to Flowcell | |
| | Activator to Flowcell | 0.5 |
| | Activatorr Phosphoramidite | 5 |
| | to Flowcell | |
| | Incubate for 25 sec | 25 |
| WASH (Acetonitrile | Acetoniirile System Flush | 4 |
| Wash Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flow cell | 5 |
| Activator Flow) | Activator + Phosphoramidite | 18 |
| | to Flowcell | |
| | Incubate for 25 sec | 25 |
| WASH (Acetonitrile | Acetonitrile System Flush | 4 |
| Wash Flow) | Acetonitrile to Flowcell | 15 |
| | N2 System Flush | 4 |
| | Acetonitrile System Flush | 4 |
| CAPPING (CapA + B, | CapA + B to Flowcell | 15 |
| 1:1, Flow) | | |
| WASH (Acetonitrile | Acetonitrile System Flush | 4 |
| Wash Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System. Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING | Deblock to Flowcell | 36 |
| (Deblock Flow) | | |
| WASH (Acetonitrile | Acetonitrile System Flush | 4 |
| Wash Flow) | N2 System Flush | 4 |
| | Acetonitrile System Flush | 4 |
| | Acetonitrile to Flowcell | 18 |
| | N2 System. Flush | 4.13 |
| | Acetonitrile System Flash | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1 M in ACN). Activator, (0.25M Benzoylthiotetrazole (“BTT”; 30-3070-xx from GlenResearch) in ACN), and Ox (0.02M 12 in 20% pyridine, 10% water, and 70% TI-IF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip.

### Example 3: Synthesis of a 100-Mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATGCTAGC CATACCATGATGATGATGATGATGAGAACCCCGCAT##TTTTTTTTTT3′, (SEQ ID NO: 715) where # denotes Thymidine-succinyl hexamide CED phosphoramidite (CLP-2244 from ChemGenes) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument.

All ten samples from the two chips were further PCR amplified using a forward (5′ATGCGGGGTTCTCATCATC3′) (SEQ ID NO: 716) and a reverse (5′CGGGATCCTTATCGTCATCG3) (SEQ ID NO: 717) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program:

98° C., 30 sec

98° C., 10 sec; 63° C., 10 sec; 72° C., 10 sec; repeat 12 cycles 72° C., 2 min

The PCR products were also run on a BioAnalyzer, demonstrating sharp peaks at the 100-mer position. Next, the PCR amplified samples were cloned, and Sanger sequenced. Table 3 summarizes the results from the Sanger sequencing for samples taken from spots 1-5 from chip 1 and for samples taken from spots 6-10 from chip 2.

TABLE 3
Sequencing results

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/525 bp | 99.93% |
| 6 | 1/615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89% of the 100-mers that were sequenced were perfect sequences with no errors, corresponding to 233 out of 262.

Table 4 summarizes error characteristics for the sequences obtained from the polynucleotides samples from spots 1-10.

TABLE 4
Error characteristics

| Sample ID/ | OSA_ | OSA_ | OSA_ | OSA_ | OSA_ | OSA_ | OSA_ | OSA_ | OSA_ | OSA_ |
|---|---|---|---|---|---|---|---|---|---|---|
| Spot no. | 0046/1 | 0047/2 | 0048/3 | 0049/4 | 0050/5 | 0051/6 | 0052/7 | 0053/8 | 0054/9 | 0055/10 |
| Total | 32 | 32 | 32 | 32 | 32 | 32 | 32 | 32 | 32 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI Match | 2500 | 2698 | 2561 | 2122 | 2499 | 2666 | 2625 | 2899 | 2798 | 2348 |
| Count | | | | | | | | | | |
| ROI | 2 | 2 | 1 | 3 | 1 | 0 | 2 | 1 | 2 | 1 |
| Mutation | | | | | | | | | | |
| ROI Multi | 0 | 0 | 0 | 0 | 0 | 0 | 0 | 0 | 0 | 0 |
| Base | | | | | | | | | | |
| Deletion | | | | | | | | | | |
| ROI Smail | 1 | 0 | 0 | 0 | 0 | 0 | 0 | 0 | 0 | 0 |
| insertion | | | | | | | | | | |
| ROI Single | 0 | 0 | 0 | 0 | 0 | 0 | 0 | 0 | 0 | 0 |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | 2349 |
| ROI Minus | MP Err: | MP Err: | MP Err: | MP Err: | MP Err: | MP Err: | MP Err: | MP Err: | MP Err: | MP Err: |
| Primer | ~1 in | ~1 in | ~1 in | ~1 in | ~1 in | ~1 in | ~1 in | ~1 in | ~1 in | ~1 in |
| Error Rate | 763 | 824 | 780 | 429 | 1525 | 1615 | 531 | 1769 | 854 | 1451 |

### Example 4: Panning and Screening for Identification of Antibodies for SARS-CoV-2 Variants

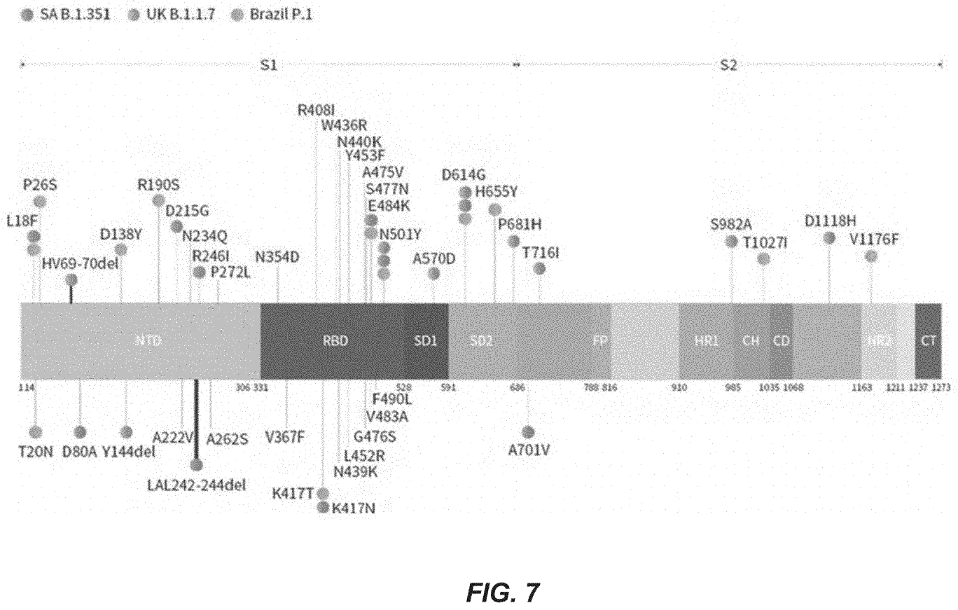

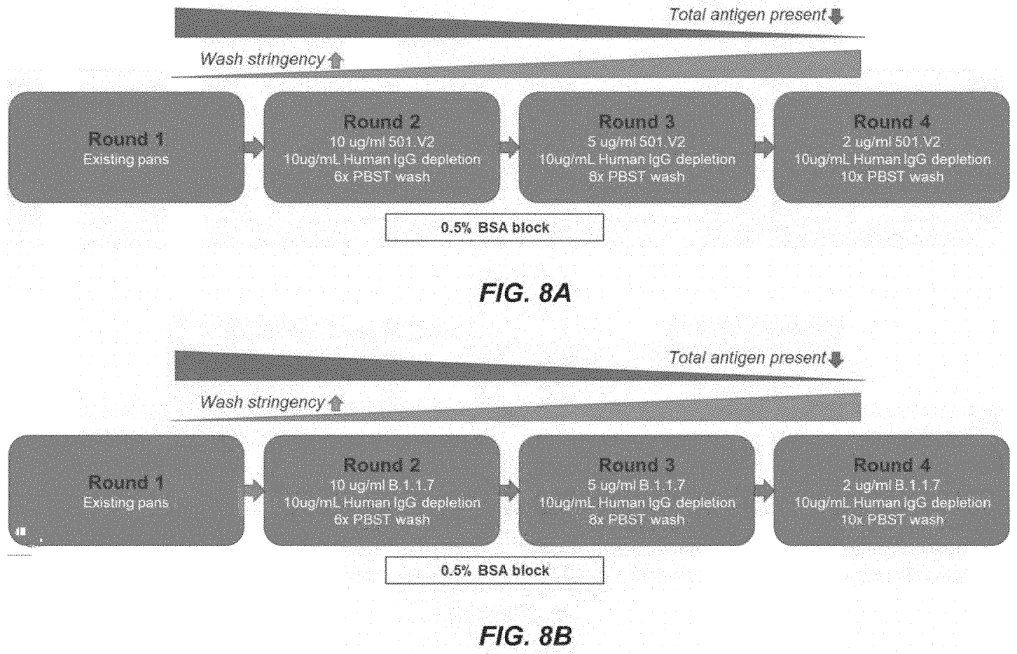

This example describes identification of antibodies for SARS-CoV-2 variants. FIG. 7 depicts different mutations found in several SARS-CoV-2 variants.

Phage displayed scFv, VHH, and Fab libraries were panned for binding to biotinylated SARS-CoV-2 S1 variants 501.V2 and B.1.1.7. Biotinylated antigen was bound to streptavidin coated magnetic beads at a density of 100 pmol antigen per mg of beads (Thermo Fisher #11206D). Phage libraries were blocked with 5% BSA in PBS. Following magnetic bead depletion for 1 hour at room temperature (RT), the beads were removed, and phage supernatant was transferred to 1 mg of antigen-bound beads in 1 mL PBS and incubated at RT with rotation for 1 hour. Non-binding clones were washed away by addition of 1 mL PBST, increasing the number of washes with each panning round. Trypsin was used to elute the phage bound to the antigen-bead complex. Phage were amplified in TG1 E. coli for the next round of selection. This selection strategy was repeated for four rounds, with successively lower amounts of antigen per round. Following all four selection rounds, 400 clones from each of round 2, 3, and 4 were selected for phage expression and phage ELISA screening. Data from the panning is seen in Tables 5-6.

TABLE 5
Panning and screening for identification of antibodies
for SARS-CoV-2 variants

| Arm | Round | # hits sequencing | # reformats |
|---|---|---|---|
| Antibody 1 | 3 | 42 | 36 |
| | 4 | 0 | |
| Antibody 2 | 3 | 1 | |
| | 4 | 0 | |
| Antibody 3 | 3 | 54 | 31 |
| | 4 | 1 | |
| Antibody 4 | 3 | 1 | |
| | 4 | 6 | |
| Antibody 5 | 3 | 0 | |
| | 4 | 0 | |
| Antibody 6 | 3 | 29 | *All were VHH |
| | 4 | 0 | |
| Antibody 7 | 3 | 59 | 45 |
| | 4 | 7 | |
| Antibody 8 | 3 | 14 | 10 |
| | 4 | 0 | |

TABLE 6
Reformat list

| Project | Target | Library | Reformat |
|---|---|---|---|
| Antibody 1 | S1 501.V2-Fc | COVID-19 | 36 IgG1 |
| | | “TAO” | |
| Antibody 3 | S1 501.V2-Fc | VHH hShuffle | 31 VHH-Fc |
| Antibody 7 | S1 B.1.1.7-Fc | VHH hShuffle | 45 VHH-Fc |
| Antibody 8 | S1 B.1.1.7-Fc | VHH | 10 VHH-Fc |
| | | Hyperimmune | |

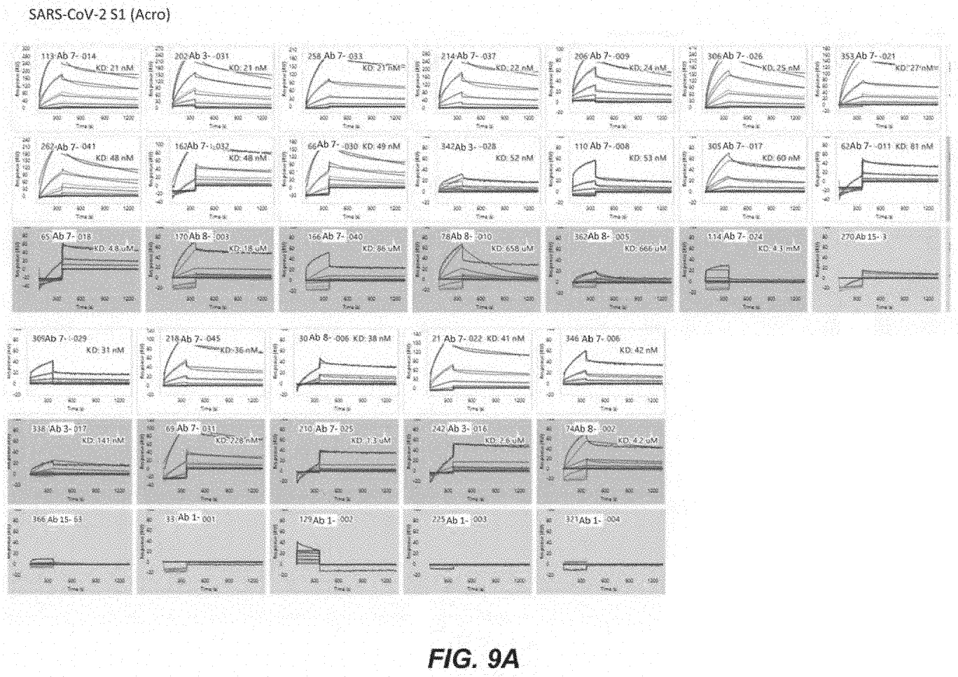

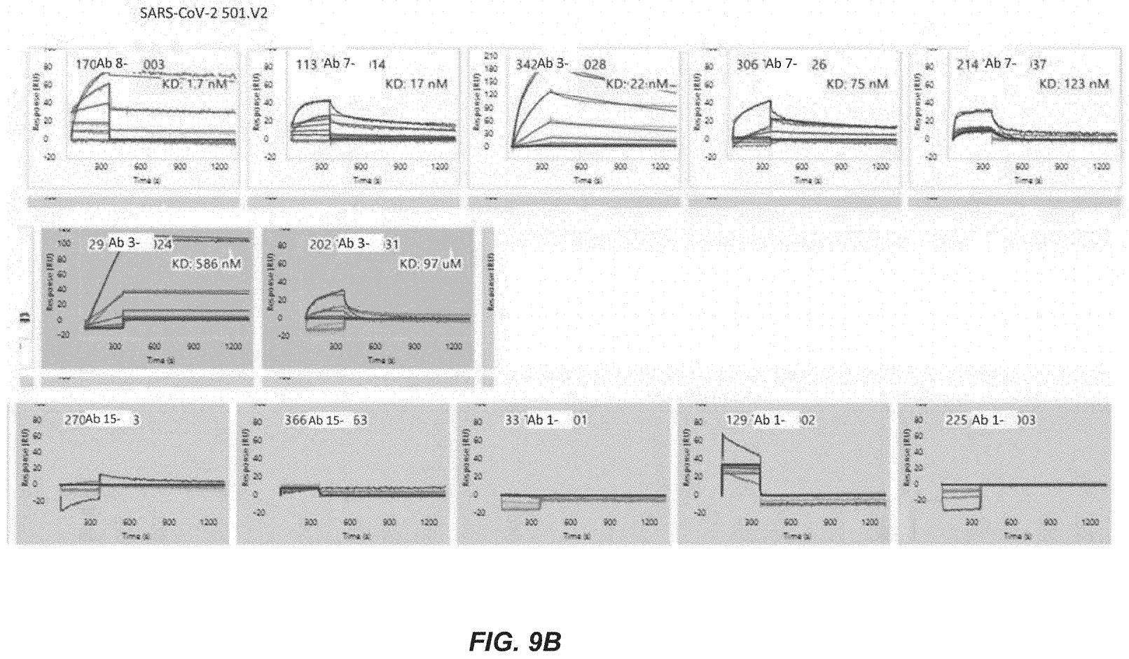

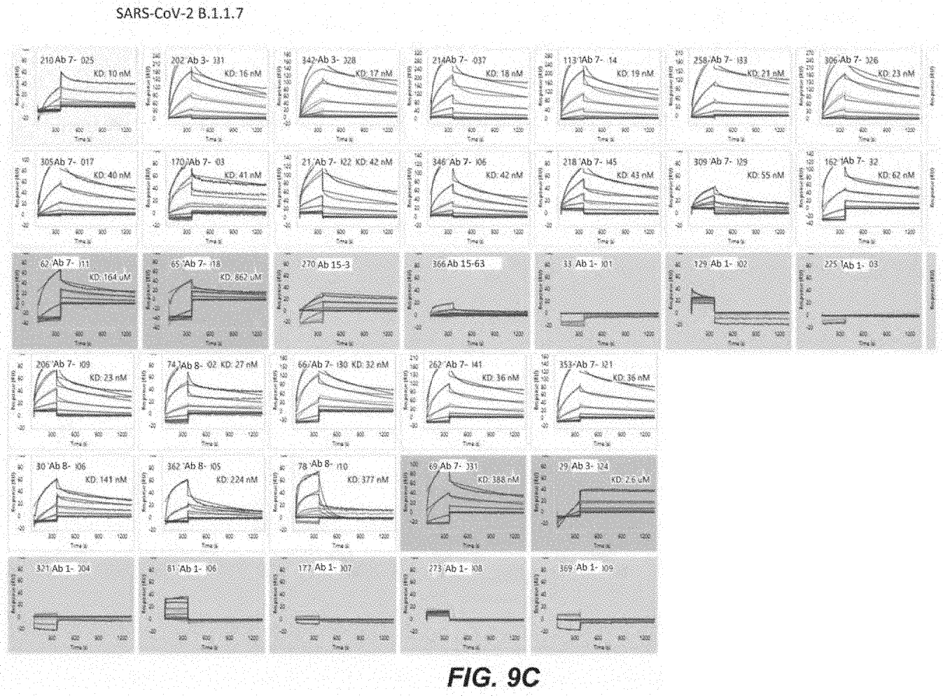

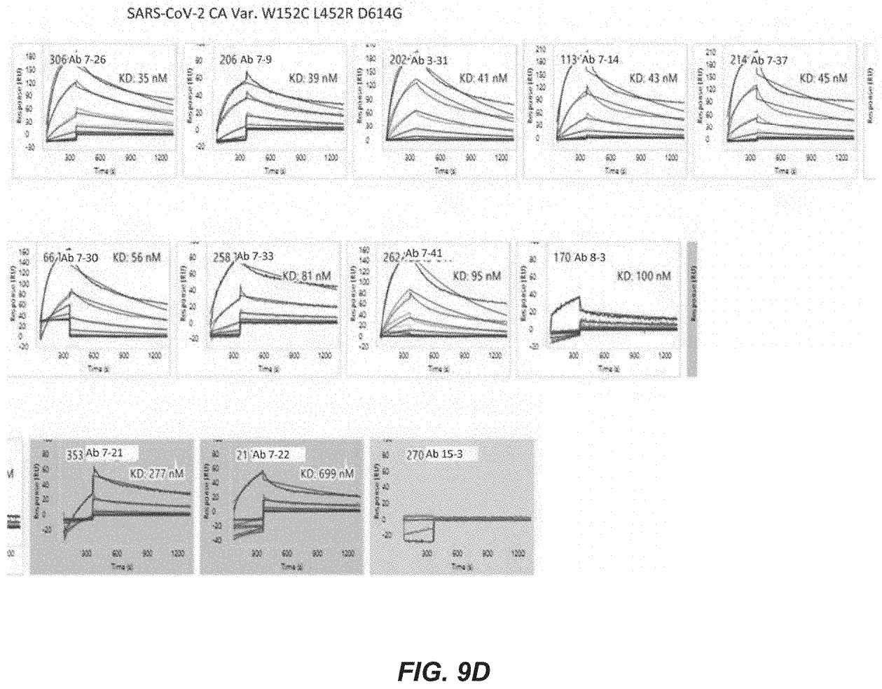

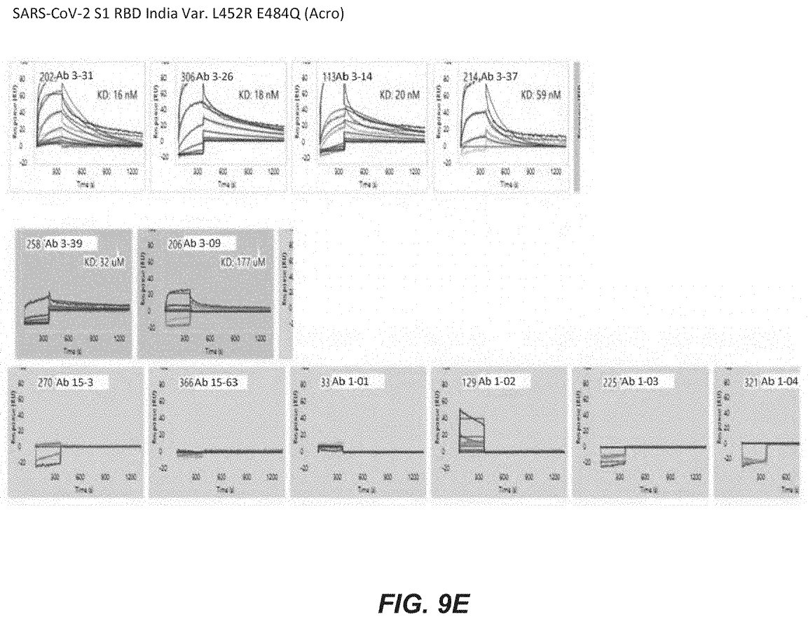

Carterra kinetics rank ordered by affinity are depicted in FIG. 9A-9E and Table 7. In Table 7, the antibodies indicated with a * are cross-reactive, including binding to the India variant mutation L452R E484Q. These are all part of the same CAADGVPEYSDYASGPVW (SEQ ID NO: 718) clonotype.

TABLE 7
Carterra kinects

| | | | | | | | Antibody | |
|---|---|---|---|---|---|---|---|---|
| | | | | | Antibody | Antibody | 178-10_ | |
| | | | | | 178-09_ | 178-08_ | His | S1 RBD |
| | | | | SARS- | His | His | CA_W152C_ | L452R |
| | | | | CoV-2 S1 | B.1.1.7 | 501.V2 | L452R_D614G | E484Q |
| | | | | (Acro) | (27080) | (27079) | (27081) | (Acro) |
| Clone | Target | Library | CDRH3 | KD (nM) | KD (nM) | KD (nM) | KD (nM) | KD (nM) |
| 7-6 | B.1.1.7 | VHH | CAAALSEVWRGS | 42.4 | 42.4 | n.b. | n.b. | n.b. |
| | | hShuffle | ENLREGYDW | | | | | |
| | | | (SEQ ID NO: 719) | | | | | |
| 3-31* | 501.V2 | VHH | CAADGVPEYSDY | 21.0 | 16.1 | 96652.0 | 41.0 | 16.2 |
| | | hShuffle | ASGPVW | | | | | |
| | | | (SEQ ID NO: 718) | | | | | |
| 7-8 | B.1.1.7 | VHH | CAADGVPEYSDY | 52.8 | n.b. | n.b. | n.b. | n.b. |
| | | hShuffle | ASGPVW | | | | | |
| | | | (SEQ ID NO: 718) | | | | | |
| 7-14* | B.1.1.7 | VHH | CAADGVPEYSDY | 20.8 | 19.2 | 16.9 | 43.1 | 20.1 |
| | | hShuffle | ASGPVW | | | | | |
| | | | (SEQ ID NO: 718) | | | | | |
| 7-26* | B.1.1.7 | VHH | CAADGVPEYSDY | 24.8 | 22.7 | 74.9 | 34.8 | 18.0 |
| | | hShuffle | ASGPVW | | | | | |
| | | | (SEQ ID NO: 718) | | | | | |
| 7-32 | B.1.1.7 | VHH | CAADGVPEYSDY | 48.5 | 62.4 | n.b. | n.b. | n.b. |
| | | hShuffle | ASGPVW | | | | | |
| | | | (SEQ ID NO: 718) | | | | | |
| 7-33 | B.1.1.7 | VHH | CAADGVPEYSDY | 21.2 | 21.1 | n.b. | 80.7 | 32467.6 |
| | | hShuffle | ASGPVW | | | | | |
| | | | (SEQ ID NO: 718) | | | | | |
| 7-37* | B.1.1.7 | VHH | CAADGVPEYSDY | 21.6 | 17.8 | 122.9 | 45.1 | 58.5 |
| | | hShuffle | ASGPVW | | | | | |
| | | | (SEQ ID NO: 718) | | | | | |
| 7-11 | B.1.1.7 | VHH | CAADRAADFFAQ | 80.6 | 164242.3 | n.b. | n.b. | n.b. |
| | | hShuffle | RDEYDW | | | | | |
| | | | (SEQ ID NO: 720) | | | | | |
| 7-30 | B.1.1.7 | VHH | CAAEVRNGSDYL | 48.7 | 32.0 | n.b. | 55.9 | n.b. |
| | | hShuffle | PIDW | | | | | |
| | | | (SEQ ID NO: 721) | | | | | |
| 3-16 | 501.V2 | VHH | CAAFDGYSGSDW | 2550.3 | n.b. | n.b. | n.b. | n.b. |
| | | hShuffle | (SEQ ID NO: 722) | | | | | |
| 7-25 | B.1.1.7 | VHH | CAAFDGYTGSDW | 1316.1 | 10.0 | n.b. | n.b. | n.b. |
| | | hShuffle | (SEQ ID NO: 723) | | | | | |
| 7-31 | B.1.1.7 | VHH | CAAQTEDSAQYI | 227.9 | 387.6 | n.b. | n.b. | n.b. |
| | | hShuffle | W | | | | | |
| | | | (SEQ ID NO: 724) | | | | | |
| 7-29 | B.1.1.7 | VHH | CAARRWIPPGPIW | 31.2 | 54.8 | n.b. | n.b. | n.b. |
| | | hShuffle | (SEQ ID NO: 725) | | | | | |
| 7-09 | B.1.1.7 | VHH | CAKEDVGKPFDW | 24.1 | 23.2 | n.b. | 38.7 | 177248.4 |
| | | hShuffle | (SEQ ID NO: 726) | | | | | |
| 7-18 | B.1.1.7 | VHH | CAKEDVGKPFDW | 4766.2 | 862396.3 | n.b. | n.b. | n.b. |
| | | hShuffle | (SEQ ID NO: 726) | | | | | |
| 7-21 | B.1.1.7 | VHH | CAKEDVGKPFDW | 27.1 | 35.6 | n.b. | 276.7 | n.b. |
| | | hShuffle | (SEQ ID NO: 726) | | | | | |
| 7-40 | B.1.1.7 | VHH | CAKEDVGKPFDW | 85612.6 | n.b. | n.b. | n.b. | n.b. |
| | | hShuffle | (SEQ ID NO: 726) | | | | | |
| 7-41 | B.1.1.7 | VHH | CAKEDVGKPFDW | 48.1 | 35.6 | n.b. | 95.5 | n.b. |
| | | hShuffle | (SEQ ID NO: 726) | | | | | |
| 7-45 | B.1.1.7 | VHH | CAKEDVGKPFDW | 36.3 | 43.4 | n.b. | n.b. | n.b. |
| | | hShuffle | (SEQ ID NO: 726) | | | | | |
| 7-22 | B.1.1.7 | VHH | CAKQDVGKPFD | 40.9 | 41.7 | n.b. | 698.5 | n.b. |
| | | hShuffle | W | | | | | |
| | | | (SEQ ID NO: 727) | | | | | |
| 3-24 | 501.V2 | VHH | CALRVRPYGQYD | n.b. | 2606.7 | 585.5 | n.b. | n.b. |
| | | hShuffle | W | | | | | |
| | | | (SEQ ID NO: 728) | | | | | |
| 8-3 | B.1.1.7 | VHH | CAREDYYDSSGY | 18366.4 | 40.5 | 1.7 | 100.5 | n.b. |
| | | hShuffle | SW | | | | | |
| | | HI | (SEQ ID NO: 729) | | | | | |
| 8-10 | B.1.1.7 | VHH | CAREGYYYDSSG | 657633.8 | 376.8 | n.b. | n.b. | n.b. |
| | | hShuffle | YPYYFDYW | | | | | |
| | | HI | (SEQ ID NO: 730) | | | | | |
| 8-2 | B.1.1.7 | VHH | CARERRYYDSSG | 4208.4 | 27.1 | n.b. | n.b. | n.b. |
| | | hShuffle | YPYYFDYW | | | | | |
| | | HI | (SEQ ID NO: 731) | | | | | |
| 7-24 | B.1.1.7 | VHH | CAREVGLYYYGS | 4257477.8 | n.b. | n.b. | n.b. | n.b. |
| | | hShuffle | GSSSRRLLGRIDY | | | | | |
| | | | YFDYW | | | | | |
| | | | (SEQ ID NO: 732) | | | | | |
| 8-06 | B.1.1.7 | VHH | CARWGPFDIW | 37.7 | 141.0 | n.b. | n.b. | n.b. |
| | | hShuffle | (SEQ ID NO: 733) | | | | | |
| | | HI | | | | | | |
| 7-17 | B.1.1.7 | VHH | CASAYNPGIGYD | 60.4 | 39.8 | n.b. | n.b. | n.b. |
| | | hShuffle | W | | | | | |
| | | | (SEQ ID NO: 734) | | | | | |
| 3-17 | 501.V2 | VHH | CATGPYRSYFAR | 141.2 | n.b. | n.b. | n.b. | n.b. |
| | | hShuffle | SYLW | | | | | |
| | | | (SEQ ID NO: 735) | | | | | |
| 3-28 | 501.V2 | VHH | CAVDLSGDAVYD | 52.2 | 16.6 | 22.0 | n.b. | n.b. |
| | | hShuffle | W | | | | | |
| | | | (SEQ ID NO: 736) | | | | | |
| 8-5 | B.1.1.7 | VHH | CAVVAMRMVTT | 665983.2 | 224.2 | n.b. | n.b. | n.b. |
| | | hShuffle | EGPDVLDVW | | | | | |
| | | HI | (SEQ ID NO: 737) | | | | | |

Tables 8A-8B depict a set of cross-reactive leads to test in the Vero E6 competition assay. Many of the cross-reactive leads are part of the same CAADGVPEYSDYASGPVW (SEQ ID NO: 718) clonotype.

TABLE 8A
Cross-reactive leads

| Clone | Target | Library | CDRH1 | CDRH2 | CDRH3 |
|---|---|---|---|---|---|
| 14-1 | Wuhan | VHH | GTFSSIGMG | VAAISWDGGATAYA | CAKEDVGKPFDW |
| | | hShuffle | (SEQ ID NO: | (SEQ ID NO: 747) | (SEQ ID NO: 726) |
| | | | 740) | | |
| 15.3 | Wuhan | VHH | FTFPSPWMG | VATINEYGGRNYA | CARVDRDFDYW |
| | | hShuffle | (SEQ ID NO: | (SEQ ID NO: 748) | (SEQ ID NO: 738) |
| | | HI | 741) | | |
| 15-63 | Wuhan | VHH | QTFNMG (SEQ | VAAIGSGGSTSYA | CWRLGNDYFDYW |
| | | hShuffle | ID NO: 742) | (SEQ ID NO: 749) | (SEQ ID NO: 739) |
| | | HI | | | |
| 3-28 | 501.V2 | VHH | FTFRRYDMG | SAISGGLAYYA (SEQ | CAVDLSGDAVYDW |
| | | hShuffle | (SEQ ID NO: | ID NO: 750) | (SEQ ID NO: 736) |
| | | | 743) | | |
| 3-31 | 501.V2 | VHH | STFSINAMG | AGTTSSGGYTNYA | CAADGVPEYSDYASGPVW |
| | | hShuffle | (SEQ ID NO: | SEQ ID NO: 751) | (SEQ ID NO: 718) |
| | | | 744) | | |
| 7-09 | B.1.1.7 | VHH | GTFSSIGMG | AAISWDGGATAYA | CAKEDVGKPFDW |
| | | hShuffle | (SEQ ID NO: | (SEQ ID NO: 752) | (SEQ ID NO: 726) |
| | | | 740) | | |
| 7-14 | B.1.1.7 | VHH | STFSINAMG | AGISRGGTTNYA (SEQ | CAADGVPEYSDYASGPVW |
| | | hShuffle | (SEQ ID NO: | ID NO: 753) | (SEQ ID NO: 718) |
| | | | 744) | | |
| 7-26 | B.1.1.7 | VHH | STFSINAMG | AGITSSGGYTNYA | CAADGVPEYSDYASGPVW |
| | | hShuffle | (SEQ ID NO: | (SEQ ID NO: 751) | (SEQ ID NO: 718) |
| | | | 744) | | |
| 7-30 | B.1.1.7 | VHH | RTFSMHAMG | ASISSQGRTNYA (SEQ | CAAEVRNGSDYLPIDW |
| | | hShuffle | (SEQ ID NO: | ID NO: 752) | (SEQ ID NO: 721) |
| | | | 745) | | |
| 7-37 | B.1.1.7 | VHH | STLSINAMG | AGTTRSGSVTNYA | CAADGVPEYSDYASGPVW |
| | | hShuffle | (SEQ ID NO: | (SEQ ID NO: 753) | (SEQ ID NO: 718) |
| | | | 746) | | |

TABLE 8B
Cross-reactive leads

| | | Antibody | Antibody | Antibody178- | |
|---|---|---|---|---|---|
| | SARS- | 178-09_ | 178-08_ | 10_His CA_ | S1 RBD |
| | CoV-2 | His | His | W152C_ | L452R |
| | S1 | B.1.1.7 | 501.V2 | L452R_ | E4S4Q |
| | (Acro) | (27080) | (27079) | D614G (27081) | (Aero) |
| Clone | KD (nM) | KD (nM) | KD (M) | KD (nM) | KD (nM) |
| 14-11 | 6.6 | t.b.d. | t.b.d. | 12.7 | t.b.d. |
| 15-3 | 31.5 | t.b.d. | t.b.d. | 26.8 | t.b.d. |
| 15-63 | 46.4 | t.b.d. | t.b.d. | n.b. | t.b.d. |
| 3-28 | 52.2 | 16.6 | 22.0 | n.b. | n.b. |
| 3-31 | 21.0 | 16.1 | 96652.0 | 41.0 | 16.2 |
| 7-09 | 24.1 | 23.2 | n.b. | 38.7 | 177248.4 |
| 7-14 | 20.8 | 19.2 | 16.9 | 43.1 | 20.1 |
| 7-26 | 24.8 | 22.7 | 74.9 | 34.8 | 18.0 |
| 7-30 | 48.7 | 32.0 | n.b. | 55.9 | n.b. |
| 7-37 | 21.6 | 17.8 | 122.9 | 45.1 | 58.5 |

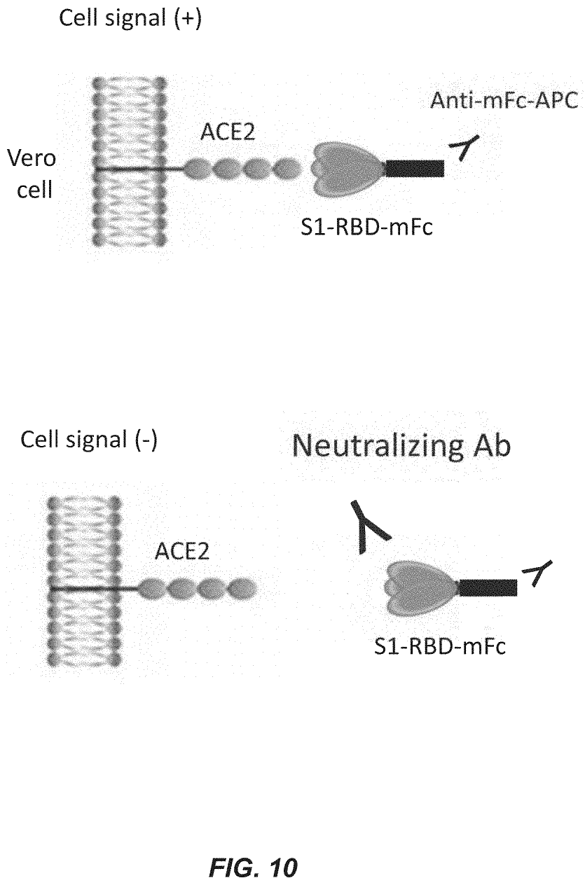

Competition ELISAs were performed on the variant antibodies. The protocol is depicted in FIG. 10. Variant antibodies with high potency in order of potency included 15-3, 15-63, 15-63 fc mutant, 14-1, 16-3, 16-4. Antibody 251-Antibody 201-1 (Lot 19898). Antibody 251-Antibody 201-1 (Lot 19442). Antibody 251-Antibody 202-76_Antibody 201-1 Antibody 201-1 and Acro mAb. SARS-CoV2 strains tested include wildtype. D614G variant, 501.V2 variant and B.1.1.7 variant.

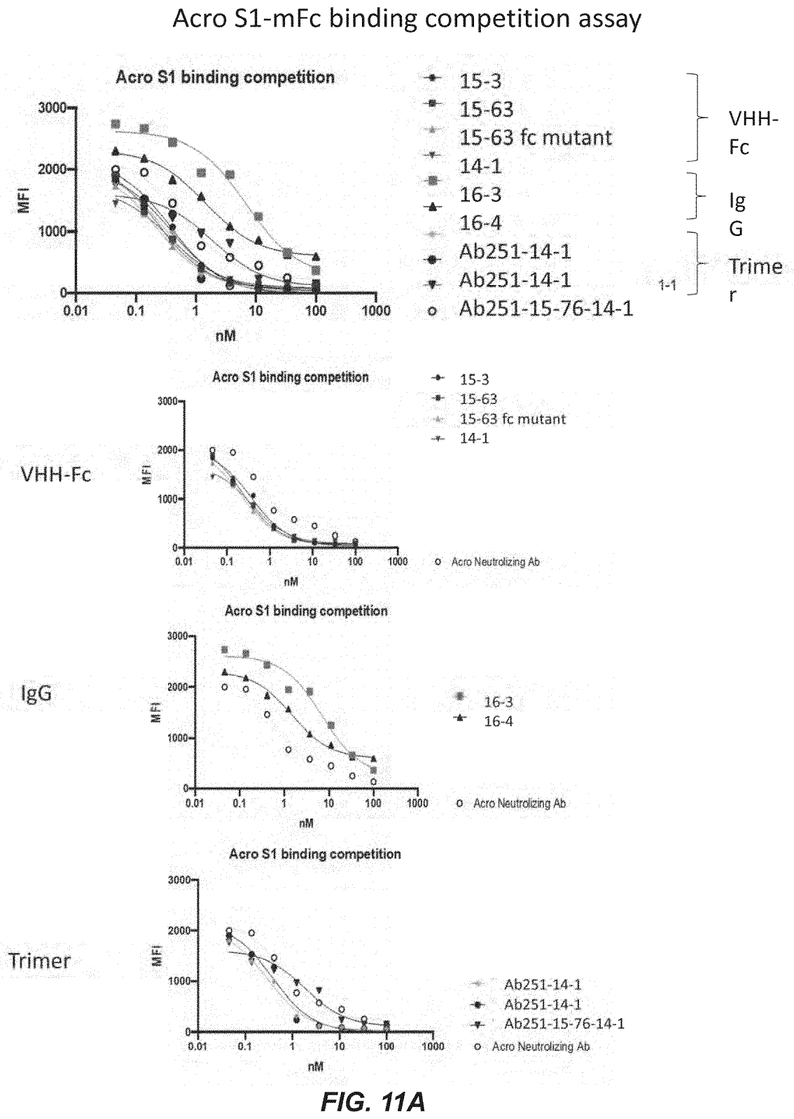

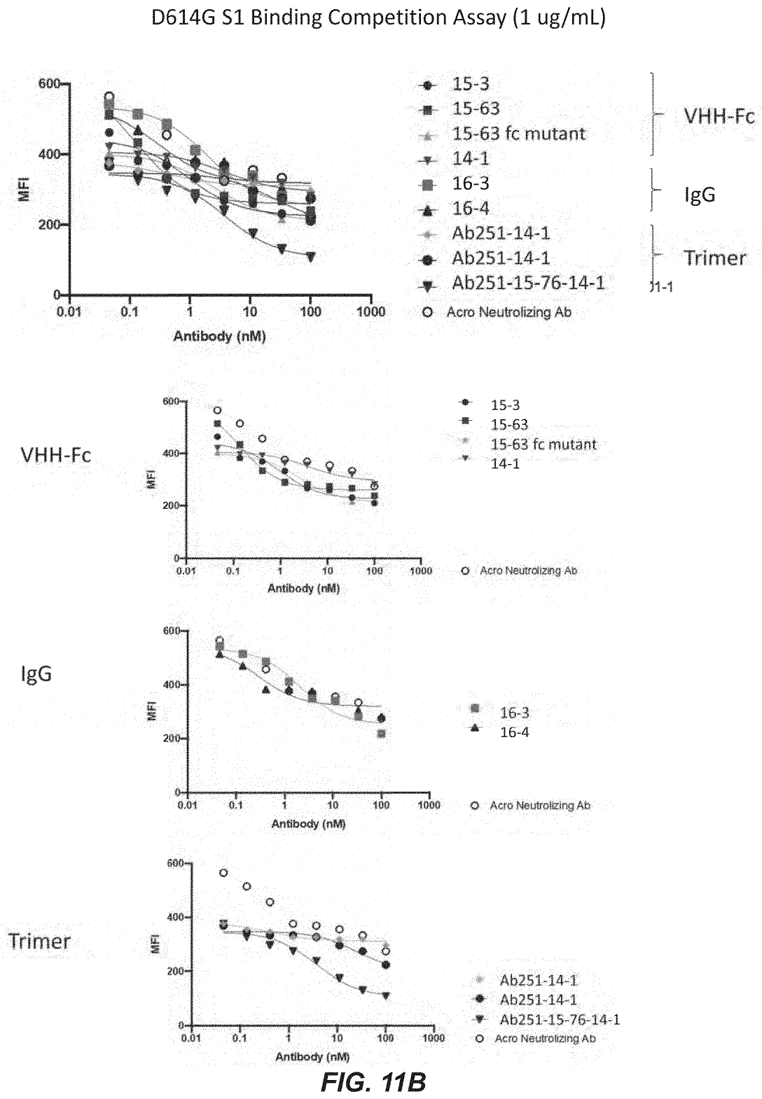

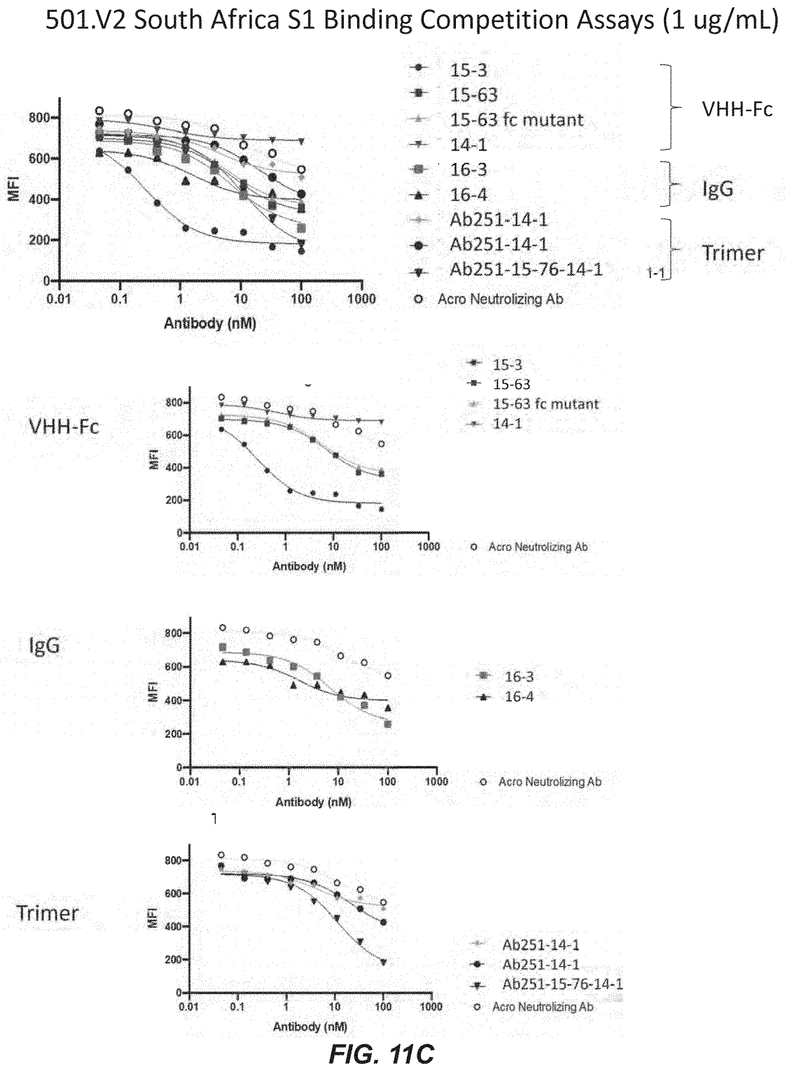

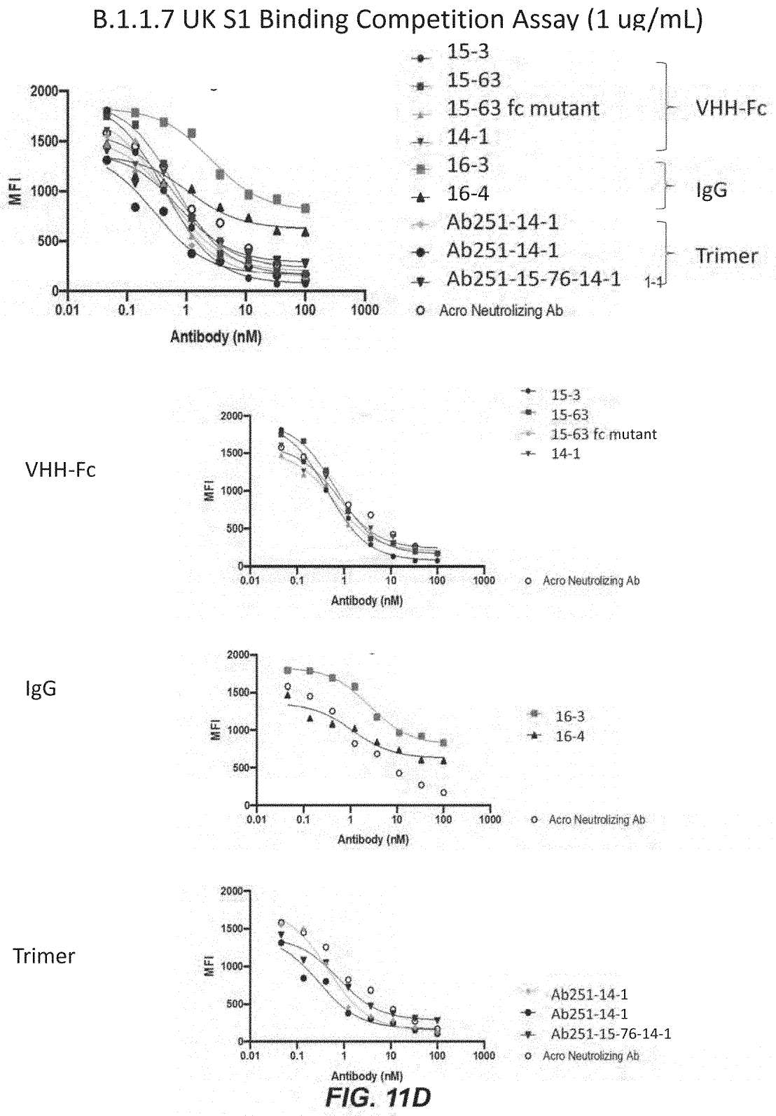

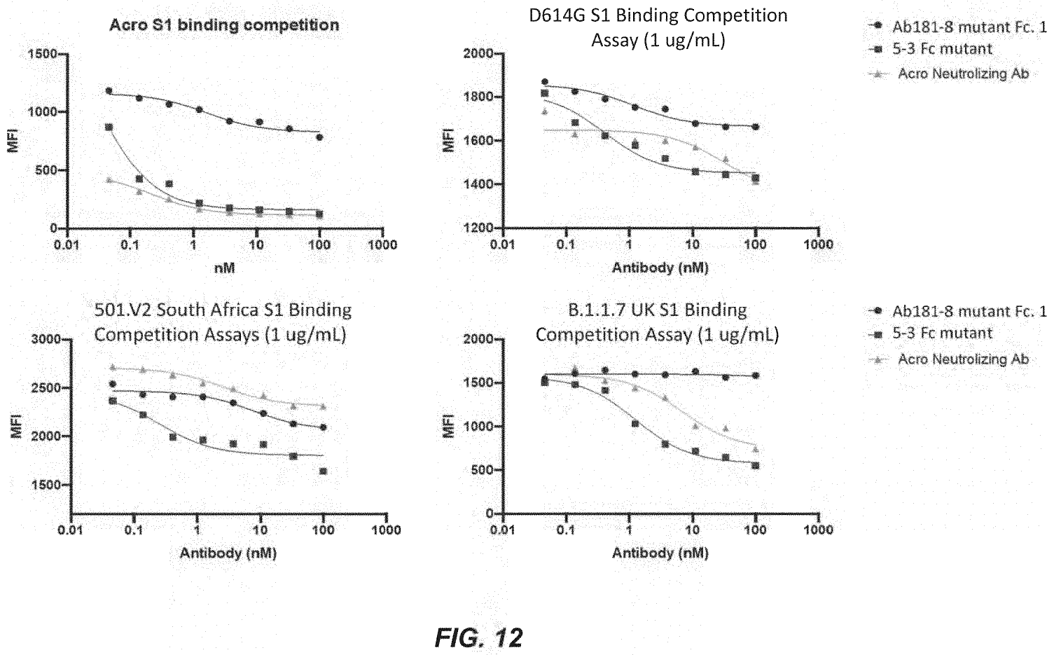

SARS-CoV-2 variant antibodies were assayed for Vero inhibition using FACS. Briefly, Vero cells stripped with Cell Stripper (˜20 minutes with 90% viability after removal). Cells were plated at 0.1×10⁶ cells per well. Stock solution of the variant antibodies were at 100 nM titrated 1:3. SARS-CoV-2 S protein RBD, SPD-C5259 were made up at 1 ug/mL. Variant antibody titrations were mixed 1:1 with 1 ug-mL S protein (50 uL IgG: 50 uL S protein). 100 uL of the mixture were added to cells and then incubated on ice for 1 hour. The cells were washed 1× followed by addition of goat anti-mouse secondary made up at 1:200. The cells were then incubated on ice for 1 hour in the dark, washed three times, and the plates were then read. Results are depicted in FIGS. 11A-11D. FIG. 12 depicts the results of an Acro S1-mFc binding competition assay comparing Antibody 181-8 mutant fc, 15-3_fc_mutant and Acro neutralizing antibody.

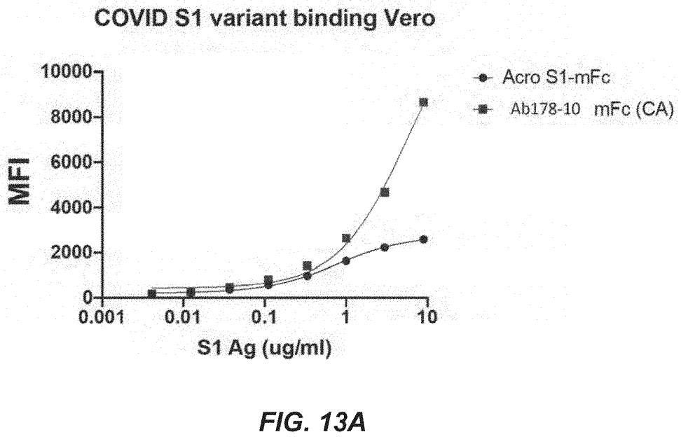

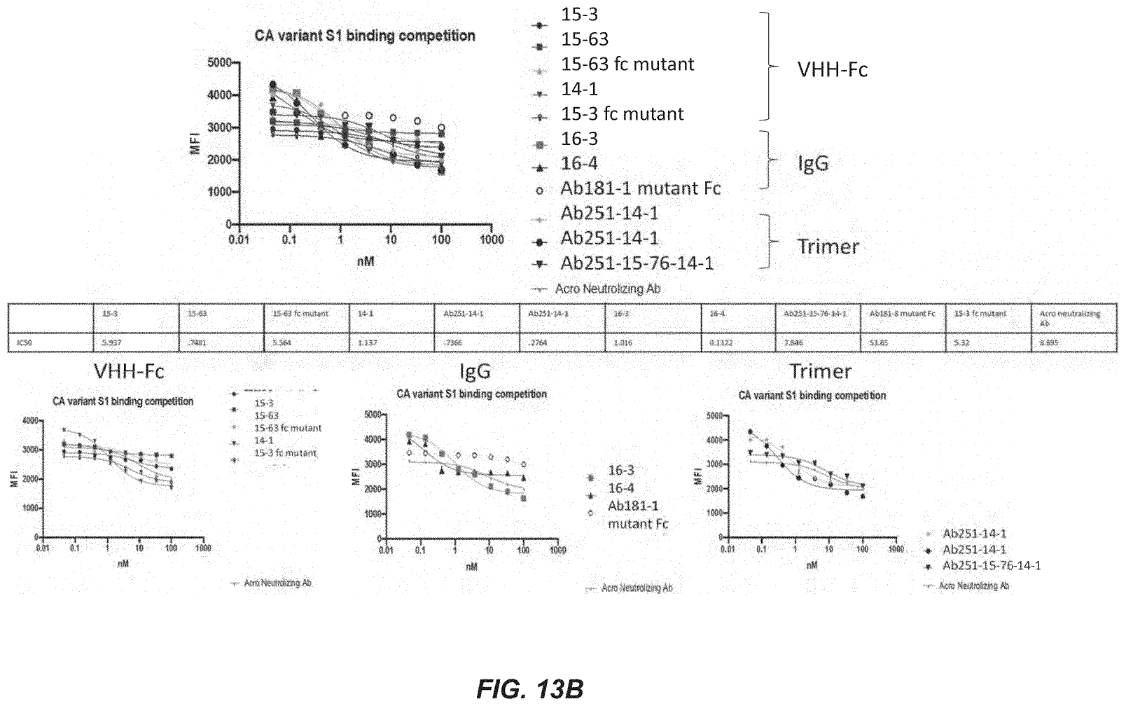

California variant S1 protein's ability to bind Vero cells was tested. As depicted in FIG. 13A, the CA si variant binds strongly to Vero cells. FIG. 13B depicts the results of a competition assay of the panel of variants against the CCA S1 spike protein.

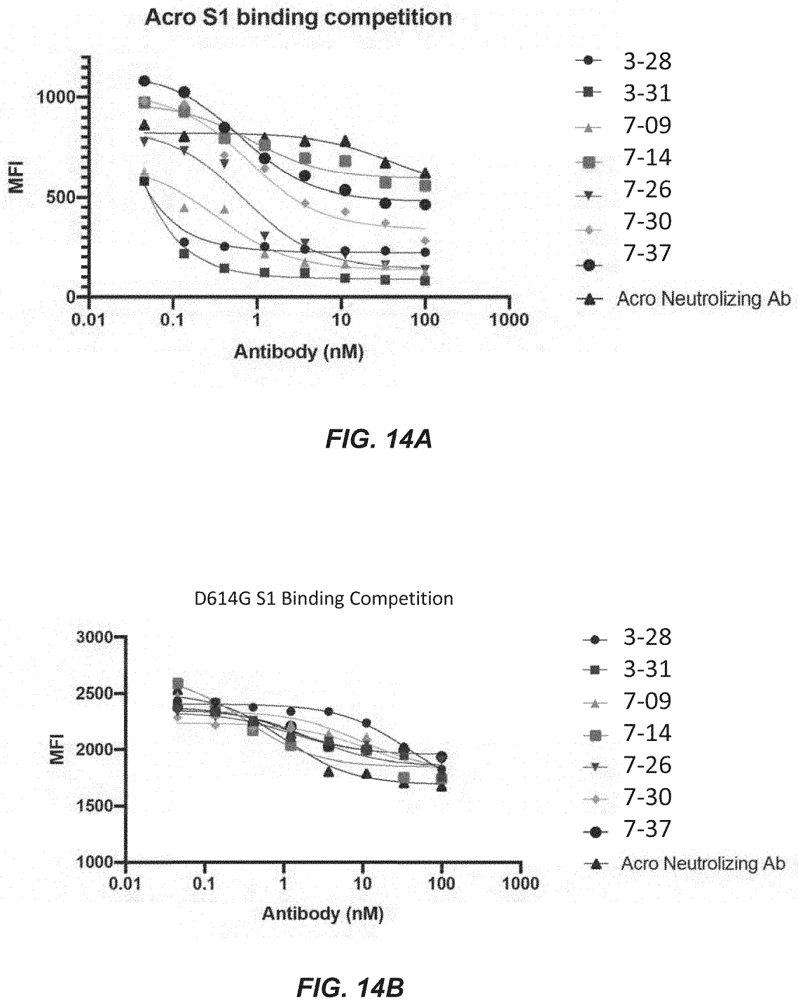

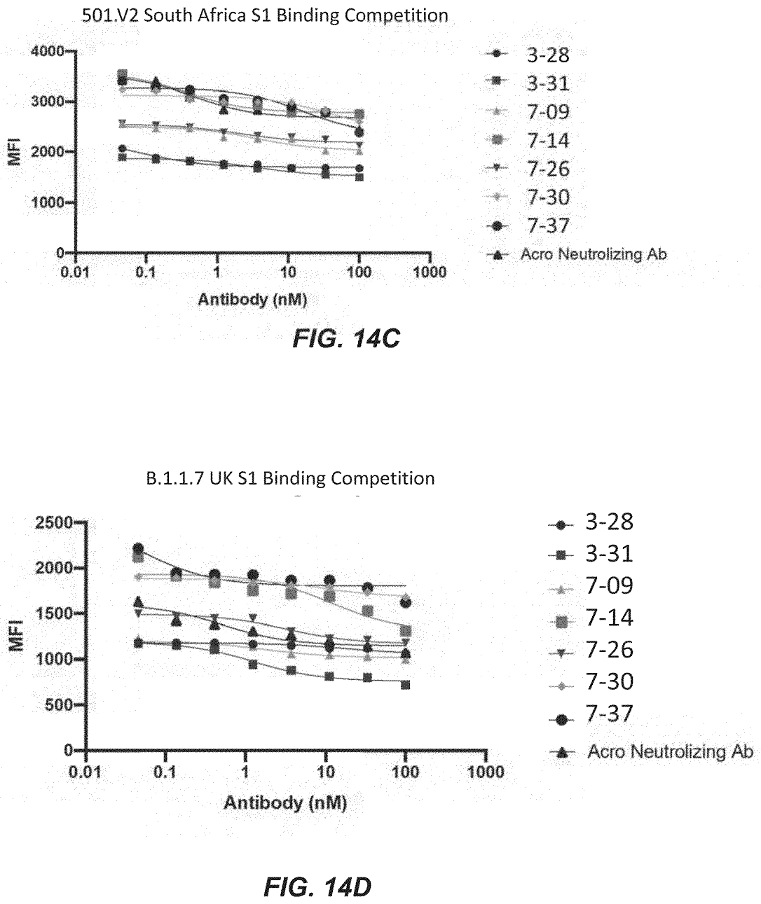

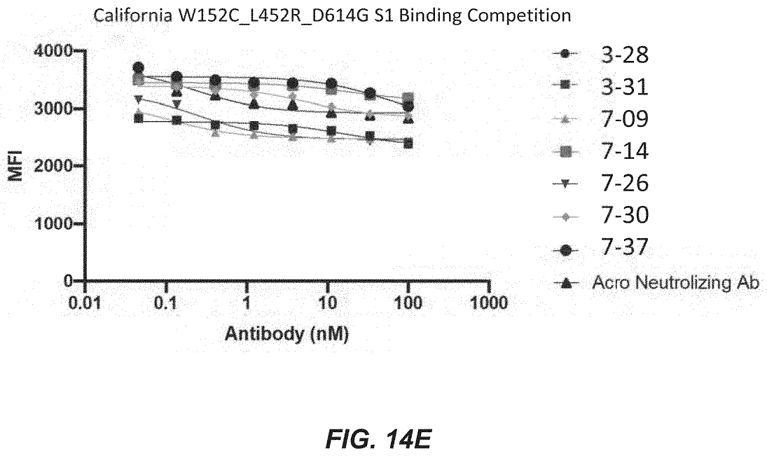

The crossreactors were also tested in a binding competition assay. SARS-CoV-2 antibody variants 3-28, 3-31, 7-9, 7-14, 7-26, 7-30, 7-37 and Acro neutralizing mAb were tested for cross-reactivity with Acro S1, Antibody 178-6 in the D614G SARS-CoV-2 variant, Antibody 178-09 in the B.1.1.7 UK variant, and Antibody 178-10 in the CA_W152C_L452R_D614G variant. Results are depicted in FIGS. 14A-14E.

### Example 5: SARS-CoV-2 Variant Panning

Phage displayed scFv, VHH, and Fab libraries were panned for binding to biotinylated SARS-CoV-2 S1 variants B.1.1.7. B.1.351, P.1, and CA (Antibody 187-10 his) S1 variants. Biotinylated antigen was bound to streptavidin coated magnetic beads at a density of 200 pmol antigen per mg of beads (Thermo Fisher #11206D). Phage libraries were blocked with 0.5% BSA in PBS. Following magnetic bead depletion for 1 hour at room temperature (RT), the beads were removed, and phage supernatant was transferred to 1 mg of antigen-bound beads in 1 ml PBS and incubated at RT with rotation for 1 hour. Non-binding clones were washed away by addition of 1 ml PBST, increasing the number of washes with each panning round. Trypsin was used to elute the phage bound to the antigen-bead complex. Phage were amplified in TG1 E. coli for the next round of selection. This selection strategy was repeated for four rounds, with successively lower amounts of antigen per round. Following all four selection rounds, 400 clones from each of round 2, 3, and 4 were selected for phage expression and phage ELISA screening. Data from the panning is seen in Table 9A and ELISA data is seen in Table 9B.

TABLE 9A
Panning Data

| Antibody | R1 | R2 | R3 | R4 |
|---|---|---|---|---|
| Antibody | B.1.1.7 | B.1.1.7 | B.1.1.7 | B.1.1.7 |
| 9 | Output: | Output: | Output: | Output |
| | 4e7 | 2e7* | 1e6 | 2.4e6 |
| Antibody | B.1.1.7 | B.1.1.7 | B.1.1.7 | B.1.1.7 |
| 10 | Output: | Output: | Output: | Output: |
| | 1e7 | 1.6e7* | 5e6 | 3.2e6 |
| Antibody | B.1.351 | B.1.351 | B.1.351 | B.1.351 |
| 11 | Output: | Output: | Output: | Output |
| | 2.6e7 | 2e7* | 3e6 | 2.4e6 |
| Antibody | B.1.351 | B.1.351 | B.1.351 | B.1.351 |
| 12 | Output: | Output: | Output: | Output: |
| | 2.8e6 | 1e8* | 5e5 | 1.8e6 |
| Antibody | P.1 | P.1 | P.1 | P.1 |
| 13 | Output: | Output: | Output: | Output: |
| | 3.2e6 | N/A* | 6e5 | 1.6e6 |
| Antibody | P.1 | P.1 | P.1 | P.1 |
| 14 | Output: | Output: | Output: | Output: |
| | 1.4e7 | N/A* | 3e5 | 1.6e6 |
| Antibody | CA | CA | CA | CA |
| 15 | Output: | Output: | Output: | Output: |
| | 3e7 | 1.4e7 | 2e6 | 1.4e7 |
| Antibody | CA | CA | CA | CA |
| 16 | Output: | Output: | Output: | Output: |
| | 8e7 | 2e7 | 1e6 | 1.4e7 |

TABLE 9B
ELISA Data

| Antibody | ELISA |
|---|---|
| 9-1 | 12.32 |
| 9-2 | 12.18 |
| 9-3 | 7.48 |
| 9-4 | 6.56 |
| 9-5 | 5.96 |
| 9-6 | 5.77 |
| 9-7 | 5.60 |
| 9-8 | 4.63 |
| 9-9 | 3.85 |
| 9-10 | 3.68 |
| 9-11 | 3.62 |
| 9-12 | 3.49 |
| 9-13 | 3.19 |
| 9-14 | 3.13 |
| 9-15 | 3.07 |
| 10-1 | 21.61 |
| 10-2 | 9.68 |
| 10-3 | 9.65 |
| 10-4 | 8.98 |
| 10-5 | 7.92 |
| 10-6 | 7.83 |
| 11-1 | 21.66 |
| 11-2 | 19.40 |
| 11-3 | 17.26 |
| 11-4 | 16.29 |
| 11-5 | 16.01 |
| 11-6 | 12.60 |
| 11-7 | 11.53 |
| 11-8 | 11.35 |
| 11-9 | 9.14 |
| 11-10 | 7.06 |
| 11-11 | 6.43 |
| 11-12 | 5.59 |
| 11-13 | 5.27 |
| 11-14 | 4.98 |
| 11-15 | 4.97 |
| 11-16 | 4.65 |
| 11-17 | 4.22 |
| 11-18 | 3.70 |
| 11-19 | 3.65 |
| 11-20 | 3.42 |
| 12-1 | 29.16 |
| 12-2 | 25.89 |
| 12-3 | 25.22 |
| 12-4 | 23.53 |
| 12-5 | 22.81 |
| 12-6 | 22.35 |
| 12-7 | 22.24 |
| 12-8 | 21.42 |
| 12-9 | 19.94 |
| 12-10 | 19.72 |
| 12-11 | 18.79 |
| 12-12 | 18.65 |
| 12-13 | 18.11 |
| 12-14 | 17.92 |
| 12-15 | 17.65 |
| 12-16 | 17.01 |
| 12-17 | 16.17 |
| 12-18 | 14.20 |
| 12-19 | 13.79 |
| 12-20 | 13.34 |
| 12-21 | 11.70 |
| 12-22 | 10.50 |
| 12-23 | 10.30 |
| 12-24 | 9.63 |
| 12-25 | 9.60 |
| 12-26 | 9.05 |
| 12-27 | 8.83 |
| 12-28 | 8.63 |
| 12-29 | 7.55 |
| 12-30 | 7.37 |
| 12-31 | 7.01 |
| 12-32 | 6.88 |
| 12-33 | 6.41 |
| 12-34 | 5.81 |
| 12-35 | 5.57 |
| 12-36 | 5.51 |
| 12-37 | 5.50 |
| 12-38 | 5.22 |
| 12-39 | 5.09 |
| 12-40 | 5.09 |
| 12-41 | 4.99 |
| 12-42 | 4.93 |
| 12-43 | 4.82 |
| 13-1 | 22.23 |
| 13-2 | 20.46 |
| 13-3 | 19.48 |
| 13-4 | 18.91 |
| 13-5 | 10.28 |

### Example 6: Exemplary Sequences

TABLE 10
Variable Domain Heavy Chain CDR Sequences

| | SEQ | | SEQ | | SEQ | |
|---|---|---|---|---|---|---|
| | ID | ID | ID | | ID | |
| Antibody | NO | NO | NO | CDRH2 | NO | CDRH3 |
| 9-1 | 1 | FTFSSYAMH | 90 | AVISYDGNHEYY | 179 | CARGYKGYYYMD |
| | | | | A | | VW |
| 9-2 | 2 | FSFNNYGMH | 91 | AVISFDGSNEYY | 180 | CAKENWLGYFDP |
| | | | | A | | W |
| 9-3 | 3 | FTFGTYAMH | 92 | AVVSTEGGTTY | 181 | CAGSYGAYFDYW |
| | | | | YA | | |
| 9-4 | 4 | FDFSDYYMH | 93 | AVISYDGSNKYY | 182 | CAREEPVYGMDV |
| | | | | A | | W |
| 9-5 | 5 | FTFGTYAMH | 94 | AVVSTEGGTTY | 183 | CAGSYGAYFDYW |
| | | | | YA | | |
| 9-6 | 6 | FTFSGYAMH | 95 | AVISYDGSNEYY | 184 | CARTNSGSYYGPF |
| | | | | A | | DYW |
| 9-7 | 7 | FTFSSYAMH | 96 | AVISYDGNHEYY | 185 | CARGYKGYYYMD |
| | | | | A | | VW |
| 9-8 | 8 | FTFSSYAMH | 97 | AVISYDGNHEYY | 186 | CARGYKGYYYMD |
| | | | | | | VW |
| 9-9 | 9 | FIFRSYAMH | 98 | AVISYDGSSKYY | 187 | CARPSSGSYFPPFD |
| | | | | A | | YW |
| 9-10 | 10 | FTFSDYGMH | 99 | AVVSYDGTTKY | 188 | CAKENWLGYFDP |
| | | | | YA | | W |
| 9-11 | 11 | FTFSNFPMH | 100 | AVISYDGSLKYY | 189 | CARYQGGYMDV |
| | | | | A | | W |
| 9-12 | 12 | FTTSRFAMH | 101 | AVISYDGSNKYY | 190 | CARDTGLGFDPW |
| | | | | A | | |
| 9-13 | 13 | FTFNNYAMH | 102 | AVISYDGNNKY | 191 | CAKTMGGSYFDA |
| | | | | YA | | FDIW |
| 9-14 | 14 | FTFSDYTMH | 103 | AVISYEGSIKYY | 192 | CARSSSGSYPSLV |
| | | | | A | | DYW |
| 9-15 | 15 | #N/A | 104 | #N/A | 193 | CARDYWVDYFKP |
| | | | | | | G |
| 10-1 | 16 | FTFSRYAMH | 105 | AVISYDGTNEYY | 194 | CARDTGLGFDPW |
| | | | | A | | |
| 10-2 | 17 | FTFSRYAMH | 106 | AVISYDGTNEYY | 195 | CARDTGLGFDPW |
| | | | | A | | |
| 10-3 | 18 | FTFSRYAMH | 107 | AVISYDGTNEYY | 196 | CARDTGLGFDPW |
| | | | | A | | |
| 10-4 | 19 | FTFSRYAMH | 108 | AVISYDGTNEYY | 197 | CARDTGLGFDPW |
| | | | | A | | |
| 10-5 | 20 | FTFSRYAMH | 109 | AVISYDGTNEYY | 198 | CARDTGLGFDPW |
| | | | | A | | |
| 10-6 | 21 | FTFSRYAMH | 110 | AVISYDGTNEYY | 199 | CARDTGLGFDPW |
| | | | | A | | |
| 11-1 | 22 | FTFGSYGMH | 111 | AVISYDGGDEYY | 200 | CARDISRYGYYGM |
| | | | | A | | DVW |
| 11-2 | 23 | FTFGTYAMH | 112 | AVVSTEGGTTY | 201 | CAGSYGAYFDYW |
| | | | | YA | | |
| 11-3 | 24 | FTFSNFAMH | 113 | AVISYDGNHEYY | 202 | CAKTNSGSYGGM |
| | | | | A | | FDYW |
| 11-4 | 25 | FTFDNYAMH | 114 | AVISDDGRNKY | 203 | CAKDNYYDSSGY |
| | | | | YA | | YGGGMDVW |
| 11-5 | 26 | FTFSSFAMH | 115 | AVISYDGSNKYY | 204 | CARSRSGSYSSYF |
| | | | | A | | DYW |
| 11-6 | 27 | FTFGTYAMH | 116 | AVVSTEGGTTY | 205 | CAGEYYDSSGSSI |
| | | | | YA | | DYW |
| 11-7 | 28 | FTFSSYAMH | 117 | AVISYDGSNQYY | 206 | CARAKGGGYRGA |
| | | | | A | | FDIW |
| 11-8 | 29 | FTFSSYAMH | 118 | AVISYDGSNTYY | 207 | CARPRGOSYWTYF |
| | | | | A | | DYW |
| 11-9 | 30 | FTFGTYAMH | 119 | AVVSTEGGTTY | 208 | CAGSYGAYFDYW |
| | | | | YA | | |
| 11-10 | 31 | FIFNNYGMH | 120 | AVISYDGSNIYY | 209 | CARDYNDGIGSYT |
| | | | | A | | GAFDSW |
| 11-11 | 32 | FTFDNYAMH | 121 | AVISYDGSNKYY | 210 | CLREGILWDVW |
| | | | | A | | |
| 11-12 | 33 | FTFSSQAMH | 122 | AVISYDGSNKYY | 211 | CAKTEGGTYGGAF |
| | | | | A | | DIW |
| 11-13 | 34 | FSFSSYGMH | 123 | AVISYDGSDKYY | 212 | CARDNYYDSSGY |
| | | | | A | | YGGGMDVW |
| 11-14 | 35 | FTFSSYSMH | 124 | AVISYDGSHKYY | 213 | CARDGWGYFDYW |
| | | | | A | | |
| 11-15 | 36 | FIFSNYGMH | 125 | AVISYDGSDKYY | 214 | CARDDYMYGFEH |
| | | | | A | | W |
| 11-16 | 37 | FTFSDHYMH | 126 | AVISYDGSNEYY | 215 | CAKDLGPAGVDY |
| | | | | A | | W |
| 11-17 | 38 | FIFSSYAMH | 127 | AVISYDGSNKYY | 216 | CARSRSGSYSSWP |
| | | | | A | | DYW |
| 11-18 | 39 | FTFGTYAMH | 128 | AVISYDGNNKY | 217 | CAKTGSGSYYSWF |
| | | | | YA | | DYW |
| 11-19 | 40 | FTFSSYAMH | 129 | AVISYDGTNDYY | 218 | CARTRGGSYFTPP |
| | | | | A | | DYW |
| 11-20 | 41 | FTFDDYAMH | 130 | AVISYDGSNKYY | 219 | CASPHSGSYWAAF |
| | | | | A | | DIW |
| 12-12-1 | 42 | FTFSYYGMH | 131 | AVTSYDGSNKY | 220 | CARPQGGSYFAAF |
| | | | | YA | | DIW |
| 12-2 | 43 | FIFRSYAMH | 132 | AVISYDGSSKYY | 221 | CARPSSGSYFPPFD |
| | | | | A | | YW |
| 12-3 | 44 | FTFSSYAMH | 133 | AVISYDGSNQYY | 222 | CAKTRTGSYFSAF |
| | | | | A | | DIW |
| 12-4 | 45 | FTFSYYGMH | 134 | AVISYDGTNDYY | 223 | CAKPHSGSYRGYF |
| | | | | A | | DYW |
| 12-5 | 46 | FTFSYYGMH | 135 | AVTSYDGSNKY | 224 | CARPKSGSYATYF |
| | | | | YS | | DYW |
| 12-6 | 47 | FIFRNYAMH | 136 | AVISYDGSNKYY | 225 | CARPRGGSYHGAF |
| | | | | A | | DIW |
| 12-7 | 48 | FTFSIYAMH | 137 | AVISYDGTNEYY | 226 | CAKSRGGSYYGAF |
| | | | | A | | DYW |
| 12-8 | 49 | FTFNNYVMH | 138 | AVISYDGTNDYY | 227 | CARGESGSYWGA |
| | | | | A | | FDYW |
| 12-9 | 50 | FTFSSYGMH | 139 | AVISYDGTTEYY | 228 | CARPSSGSYLGFF |
| | | | | A | | DYW |
| 12-10 | 51 | FIFRSYAMH | 140 | AVISYDOSIKYY | 229 | CARTRGGSYYGAF |
| | | | | A | | DYW |
| 12-11 | 52 | FSFGGYGMH | 141 | AVISYDGSNEYY | 230 | CAKSYSGSYSSYE |
| | | | | A | | DYW |
| 12-12 | 53 | FAFSSHAMH | 142 | AVISYDGSNKYY | 231 | CAKAYSGSYMGY |
| | | | | A | | FDYW |
| 12-13 | 54 | FSFSTYGMH | 143 | AVISYDGSNKYY | 232 | CARPLSGSYWSWF |
| | | | | A | | DPW |
| 12-14 | 55 | FTFSSYSMH | 144 | AVISYDGSNKYY | 233 | CARGKGGGYYSSF |
| | | | | A | | DFW |
| 12-15 | 56 | FSFGGYGMH | 145 | AVISYDGSNKYY | 234 | CARPYSGSYISWF |
| | | | | A | | DYW |
| 12-16 | 57 | FIFRSYAMH | 146 | AVISYDGSSKYY | 235 | CARTLGGSYFAAF |
| | | | | A | | DIW |
| 12-17 | 58 | FTTGSYGMH | 147 | AVISYDGNHEYY | 236 | CARPHSGSYTAYF |
| | | | | A | | DYW |
| 12-18 | 59 | FTFSSYAMH | 148 | AVISYDGSNQYY | 230 | CARGYGGSYSYFD |
| | | | | A | | YW |
| 12-19 | 60 | FAFSSYAMH | 149 | AVISYDGTYEYY | 238 | CARSLGGSYFSGM |
| | | | | A | | DVW |
| 12-20 | 61 | FSFGGYGMH | 150 | AVSYDGSNKYY | 239 | CARSKGGSYYGPF |
| | | | | A | | DYW |
| 12-21 | 62 | FSFGGYGMH | 151 | AVISYDGSNKYY | 240 | CARPKGGNYWNA |
| | | | | A | | FDIW |
| 12-22 | 63 | FTFSSYGMH | 152 | AVISYDGNHEYY | 241 | CARPKSGSYVSYF |
| | | | | A | | DYW |
| 12-23 | 64 | FIFSSYAMH | 153 | AVISYDGSNKYY | 242 | CARPRGGNYLNYF |
| | | | | A | | DYW |
| 12-24 | 65 | FTFSNFPMH | 154 | AVISYDGNNKY | 243 | CAKDHGDHYFDY |
| | | | | YA | | W |
| 12-25 | 66 | FTTSSYAMH | 155 | AVISYDGSNQYY | 244 | CARDKGGSYYGPF |
| | | | | A | | DYW |
| 12-26 | 67 | FTFSNYAMH | 156 | AVISYDGSNEYY | 245 | CAKSGSGSYFSPF |
| | | | | A | | DYW |
| 12-27 | 68 | FSFGGYGMH | 157 | AVISYDGSTKYY | 246 | CARPRGGSYKDAF |
| | | | | A | | DIW |
| 12-28 | 69 | FTFSSYAMH | 158 | AVISYDGTNEYY | 247 | CARAHGGSYFSG |
| | | | | A | | MDVW |
| 12-29 | 70 | FSFSNYGMH | 159 | AVISYDGNNKY | 248 | CARSKGGSYYGPF |
| | | | | YA | | DDW |
| 12-30 | 71 | FTFSGYAMH | 160 | AVISYDGSNKYY | 249 | CARSRGGSYYAPF |
| | | | | A | | DYW |
| 12-31 | 72 | #N/A | 161 | #N/A | 250 | CARPLGGSYFAAF |
| | | | | | | DIW |
| 12-32 | 73 | FTFGTYAMH | 162 | AVISYDGNNKY | 251 | CAKTMSGSYFSAF |
| | | | | YA | | DIW |
| 12-33 | 74 | FTFSSYAMH | 163 | AVISYDGSNQYY | 252 | CARPHGGNYFDW |
| | | | | A | | FDPW |
| 12-34 | 75 | FIFRSYAMH | 164 | AVISYDGSSKYY | 253 | CARPSGGSYFDPF |
| | | | | A | | DYW |
| 12-35 | 76 | FTFSSSSMH | 165 | AVISYDGSNKYY | 254 | CAKVDSGSYVGYP |
| | | | | A | | DYW |
| 12-36 | 77 | FSFNNYGMH | 166 | AVISYDGSNDYY | 255 | CARPNSGSYSNYF |
| | | | | A | | DYW |
| 12-37 | 78 | FTFSSYAMH | 167 | AVISYDGSNQYY | 256 | CARSRSGSYLAYF |
| | | | | A | | DYW |
| 12-38 | 79 | FTFSSYAMH | 168 | AVISYDGSNQYY | 257 | CARAAGGSYSSWF |
| | | | | A | | DPW |
| 12-39 | 80 | FTFSSYAMH | 169 | AVISYDGNHEYY | 58 | CARAHSGSYFSHF |
| | | | | A | | DYW |
| 12-40 | 81 | FTFSSYAMH | 170 | AVISYDGSNTYY | 259 | CARPTSGSYFSWF |
| | | | | A | | DPW |
| 12-41 | 82 | FIFSSYAMH | 171 | AVISYDGSNKYY | 260 | CARPNSGSYWGPF |
| | | | | A | | DYW |
| 12-42 | 83 | FTPGSYGMH | 172 | AVISYDGSHKYY | 261 | CARALGGNYYYF |
| | | | | A | | DYW |
| 12-43 | 84 | FIFSSYGMH | 173 | AVISYDGSNEYY | 262 | CARPRSGSYLSAF |
| | | | | A | | DYW |
| 13-1 | 85 | FTTSSYSMH | 174 | AVISYDGRNQY | 263 | CAKGYGGNYYYM |
| | | | | YA | | DGW |
| 13-2 | 86 | FTFSSYAMH | 175 | AVISYDGNNKY | 264 | CARTYGGSYYSAF |
| | | | | YA | | DYW |
| 13-3 | 87 | FSFNNHAMH | 176 | AVISYDGSDKYY | 265 | CARNLLRGYGMD |
| | | | | A | | VW |
| 13-4 | 88 | FAFDDYAMH | 177 | AVISYDGSNKYY | 266 | CATLGYGDYPDY |
| | | | | | | W |
| 13-5 | 89 | FIFRSYAMH | 178 | AVISYDGSSKYY | 267 | CARPLGGGYQDAF |
| | | | | A | | DIW |

TABLE 11
Variable Domain Light Chain CDR Sequences

| | | | SEQ | | SEQ | |
|---|---|---|---|---|---|---|
| | SEQ | | ID | | ID | |
| Antibody | ID NO | CDRL1 | NO | CDRL2 | NO | CDRL3 |
| 9-1 | 268 | RASQGVSNYLA | 357 | DASNRAT | 446 | CQQRYSWVTF |
| 9-2 | 269 | RASQSVSSSLA | 358 | DASNRAT | 447 | CQQRINWPRSF |
| 9-3 | 270 | RASQSVNSYLA | 359 | DVSNRAT | 448 | COQFSNWPTF |
| 9-4 | 271 | RASQSVGTSLA | 360 | GASNRAT | 449 | CQQRSNWQPF |
| 9-5 | 029 | RATQYVNSYLA | 361 | DVSNRAT | 450 | CQQFSNWPTF |
| 9-6 | 273 | RASQSVGTSLA | 362 | GASNRAT | 451 | CQLRSNWYTF |
| 9-7 | 274 | RASQGVSNYLA | 363 | DASNRAT | 452 | CQQRYSWVTF |
| 9-8 | 275 | RASQGVSNYLA | 364 | DASNRAT | 453 | CQQRYSWVTF |
| 9-9 | 276 | RASQSVDSRLA | 365 | DTSNRAT | 454 | CQQRSTWPPVF |
| 9-10 | 277 | RASQSVRHHLA | 366 | DASNRAT | 455 | CQQRTDWPRAF |
| 9-11 | 278 | RASQSVGNFLA | 367 | DASNRAT | 456 | CQQSSTWPLTF |
| 9-12 | 279 | RASESISTYLA | 368 | DASNRAT | 457 | CQQRSGLITE |
| 9-13 | 280 | RASQSVGDPLA | 369 | DTSNRAT | 458 | CQQRSNLTP |
| 9-14 | 281 | RASQTIRNSLN | 370 | ASSSLQS | 459 | CQQTHSIPKTF |
| 9-15 | 282 | RASQSVSSSLA | 371 | DASNRAT | 460 | CQQRINWPRSP |
| 10-1 | 283 | RASQDVSTYLA | 371 | DASNRAT | 461 | CQQRRDWPQTF |
| 10-2 | 284 | RASQDVSTYLA | 373 | DASNRAT | 462 | CQQRRDWPQTF |
| 10-3 | 285 | RASQDVSTYLA | 374 | DASNRAT | 463 | CQQRRDWPQTF |
| 10-4 | 286 | RASQDVSTYLA | 375 | DASNRAT | 464 | CQQRRDWPQTF |
| 10-5 | 287 | RASQDVSTYLA | 376 | DASNRAT | 465 | CQQRRDWPQTF |
| 10-6 | 288 | RASQDVSTYLA | 377 | DASNRAT | 466 | CQQRRDWPQTF |
| 11-1 | 289 | RASQSLGSFLA | 378 | DASNRAT | 467 | CQQRALWPRLTF |
| 11-2 | 290 | RASQSVNSYLA | 379 | DVSNRAT | 468 | CQQFSNWPTF |
| 11-3 | 291 | RASQNIGNHLA | 380 | DASNRAT | 469 | CQQRDNGPPEGTF |
| 11-4 | 292 | RASQSVGSYLA | 381 | DAVNRAT | 470 | CQQRFTWPTTF |
| 11-5 | 293 | RASQSITDYLA | 382 | DASNRAT | 471 | CHQRNNWPPTF |
| 11-6 | 294 | RASQSVDSSLA | 383 | DASNRAT | 472 | CQQQSNWPGTF |
| 11-7 | 295 | RASQSIGSYLA | 384 | DGSNRAT | 473 | CQQRTNWPLPSP |
| 11-8 | 296 | RASQTVTNYLA | 385 | DTSNRAT | 474 | CQHRDDWPPTF |
| 11-9 | 297 | RASQSVSYYLA | 386 | DSSNRAT | 475 | CQQRSNWQGNF |
| 11-10 | 298 | RASQSVSTSLA | 387 | DATNRAT | 476 | CQQHYSWPLTF |
| 11-11 | 299 | RASHNINNFLA | 388 | DTSNRAT | 477 | CQQGRNWPPSSF |
| 11-12 | 300 | RASQSVGTSLA | 389 | GASNRAT | 478 | CQERSNWPDTF |
| 11-13 | 301 | RASQSVSSQLA | 390 | DTSNRAT | 479 | CQQRYNWPSTF |
| 11-14 | 302 | RASQSVDSRLA | 391 | DASNRAT | 480 | CQQRTNLPPSITF |
| 11-15 | 303 | RASQSVGSYLA | 392 | DAVNRAT | 481 | CQQRSDSITF |
| 11-16 | 304 | #N/A | 393 | #N/A | 482 | #N/A |
| 11-17 | 305 | RASQNIGSHLA | 394 | DVSNRAT | 483 | CQQRDYWPPYTF |
| 11-18 | 306 | RASQSLTSYLA | 395 | DASNRAT | 484 | CQQRHYWPPITF |
| 11-19 | 307 | RASQSIGSYLA | 396 | DASNRAT | 485 | CQQRDSWPHTF |
| 11-20 | 308 | RASQSVGSYLA | 397 | DAVNRAT | 486 | CQQRSLWPF |
| 12-1 | 309 | RASQSVSSHLA | 398 | DVSNRAT | 487 | CQQRDTFTF |
| 12-2 | 310 | RASQSVDSRLA | 399 | DTSNRAT | 488 | COQRSTWPPVF |
| 12-3 | 311 | RASQSVGDFLA | 400 | DTSNRAT | 489 | CQYRSNFIF |
| 12-4 | 312 | RASQSVGSHLA | 401 | DASNRAT | 490 | CQQISNWPLTF |
| 12-5 | 313 | RASQNVGQSLA | 402 | DASNRAT | 491 | CQQRENWPPTF |
| 12-6 | 314 | RASQSLGNYLA | 403 | DSSNRAT | 492 | CQQRNWPYTF |
| 12-7 | 315 | RASQSLGNYLA | 404 | DSSNRAT | 493 | CQQRTDWPPSF |
| 12-8 | 316 | RASQNIGNHLA | 405 | DVSNRAT | 494 | CQQRKSWPPFTF |
| 12-9 | 317 | RASQSVSTSLA | 406 | DATNRAT | 495 | CQRRTDWPPTF |
| 12-10 | 318 | RASQSVNSDLA | 407 | DASNRAT | 496 | CQQRTDWPPATF |
| 12-11 | 319 | RASQSVGSYLA | 408 | DAVNRAT | 497 | CQQRFTWPTTF |
| 12-12 | 320 | RASQSVSSSLA | 409 | DASNRAT | 498 | CQHRDDWPPTF |
| 12-13 | 321 | RASQSVGSYLA | 410 | DAVNRAT | 499 | CQQRNSWPPATF |
| 12-14 | 322 | RASQSVGSYLA | 411 | DAVNRAT | 500 | CQQVSNWPLTF |
| 12-15 | 323 | RASQSVSSHLA | 412 | DVSNRAT | 501 | CQVRSDWPPLTF |
| 12-16 | 324 | RASQSLDSYLA | 413 | DVSNRAT | 502 | CQQRRGWPPVTF |
| 12-17 | 325 | RASQSVSKFLA | 414 | DASNRAT | 503 | CHQHSDWPLTF |
| 12-18 | 326 | RASQSIGGSLA | 415 | DASNRAT | 504 | CQQRYSYFTF |
| 12-19 | 327 | RASQSISRYLA | 416 | DVSNRAT | 505 | CQQSSNWPLFTP |
| 12-20 | 328 | RASQSLGNYLA | 417 | DSSNRAT | 506 | CQQRNTWPGVTF |
| 12-21 | 329 | RASQSVNSDLA | 418 | DASNRAT | 507 | CQERSLF |
| 12-22 | 330 | RASQSVRHHLA | 419 | DASNRAT | 508 | CQERSDWPITF |
| 12-23 | 331 | RASQSVDSRLA | 420 | DASNRAT | 509 | CQQRSTWPPVF |
| 12-24 | 332 | RASQSFGDSLA | 421 | DASNRAT | 510 | CQQRSIPITF |
| 12-25 | 333 | RASQSVNSYLA | 422 | DVSNRAT | 511 | CQERGNWPPFTF |
| 12-26 | 334 | RASQSVSTSLA | 423 | DISNRAT | 512 | CQQRRSGLTF |
| 12-27 | 335 | RASDTVSSYLA | 424 | DTSNRAT | 513 | CQQRASWPLSF |
| 12-28 | 336 | RASQSVRHHLA | 425 | DASNRAT | 514 | CQQSGSWPLTF |
| 12-29 | 337 | RASQIISSYLA | 426 | DTSNRAT | 515 | CQVRSNWPPLTF |
| 12-30 | 338 | RASHNIGTYLA | 427 | DVSNRAT | 516 | CQQRADWPQTF |
| 12-31 | 339 | #N/A | 428 | #N/A | 517 | #N/A |
| 12-32 | 340 | RASQSIGSYLA | 429 | DVSNRAT | 518 | CQQRDSFTF |
| 12-33 | 341 | RASQDVSTYLA | 430 | DASNRAT | 519 | CQQRAYWPGTF |
| 12-34 | 342 | RASQSVGNFLA | 431 | DASNRAT | 520 | CQHRRLLTF |
| 12-35 | 343 | RASQRVSSYLA | 432 | DAFNRAT | 521 | CQQRFDWPLTF |
| 12-36 | 344 | RASQGISTYLA | 433 | DASNRAT | 522 | CFFRRRWPPTF |
| 12-37 | 345 | RASESVSESLA | 434 | DASNRAT | 523 | CQQRTHGVTF |
| 12-28 | 346 | RASQSVSTSLA | 435 | DATNRAT | 524 | CFQRQKWPLTF |
| 12-39 | 347 | RASESISTYLA | 436 | DASNRAT | 525 | CQQRRNSLTF |
| 12-40 | 348 | RASQSVNSDLA | 437 | DASNRAT | 526 | CFQRSTWSPLTF |
| 12-41 | 349 | RASQNVGQSLA | 438 | DASNRAT | 527 | CQLRTNWPPVTF |
| 12-42 | 350 | RASQSVDSRLA | 439 | DTSNRAT | 528 | CQQRSSNWTF |
| 12-43 | 351 | RASQSVGKSLA | 440 | DTSNRAT | 529 | CQQRGSFPLTF |
| 13-1 | 352 | RASQSVGDFLA | 441 | DTSNRAT | 530 | CQQRSIRGTF |
| 13-2 | 353 | RASDTVSSYLA | 442 | DTSNRAT | 531 | CQQRGGWPPAF |
| 13-3 | 354 | RASQSIGDYLN | 443 | EASSLQS | 532 | CLHTYLPPYSF |
| 13-4 | 355 | RASQSITRVT | 444 | AASSLQS | 533 | CFQTYNPPHTF |
| 13-5 | 356 | RASQSIGSYLA | 445 | DVSNRAT | 534 | CQQRHHWPPVTF |

TABLE 12
Variable Domain Heavy Chain Sequences

| | SEQ ID | |
|---|---|---|
| Antibody | NO | VH |
| 9-1 | 535 | QVQLVESGGGVVQPGRSLRLSCAASGFTFSSYAMHWVRQAPGKGL |
| | | EWVAVISYDGNHEYYADSVKGRFTISRDNSKNTLYLQMNSLRAED |
| | | TAVYYCARGYKGYYYMDVWGQGTLVTVSS |
| 9-2 | 536 | QVQLVESGGGVVQPGRSLRLSCAASGFSFNNYGMHWVRQAPGKG |
| | | LEWVAVISFDGSNEYYADSVKGRFTISRDNSKNTLYLQMNSLRAED |
| | | TAVYYCAKENWLGYFDPWGQGTLVTVSS |
| 9-3 | 537 | QVQLVESGGGVVQPGRSLRLSCAASGFTFGTYAMHWVRQAPGKG |
| | | LEWVAVVSTEGGTTYYADSVKGRFTISRDNSKNTLYLQMNSLRAE |
| | | DTAVYYCAGSYGAYFDYWGQGTLVTVSS |
| 9-4 | 538 | QVQLVESGGGVVQPGRSLRLSCAASGFDFSDYYMHWVRQAPGKG |
| | | LEWVAVISYDGSNKYYADSVKGRFTISRDNSKNTLYLQMNSLRAE |
| | | DTAVYYCAREEPVYGMDVWGQGTLVTVSS |
| 9-5 | 539 | QVQLVESGGGVVQPGRSLRLSCAASGFTFGTYAMHWVRQAPGKG |
| | | LEWVAVVSTEGGTTYYADSVKGRFTISRDNSKNTLYLQMNSLRAE |
| | | DTAVYYCAGSYGAYFDYWGQGTLVTVSS |
| 9-6 | 540 | QVQLVESGGGVVQPGRSLRLSCAASGFTFSGYAMHWVRQAPGKGL |
| | | EWVAVISYDGSNEYYADSVKGRFTISRDNSKNTLYLQMNSLRAEDT |
| | | AVYYCARTNSGSYYGPFDYWGQGTLVTVSS |
| 9-7 | 541 | QVQLVESGGGVVQPGRSLRLSCAASGFTFSSYAMHWVRQAPGKGL |
| | | EWVAVISYDGNHEYYADSVKGRFTISRDNSKNTLYLQMNSLRAED |
| | | TAVYYCARGYKGYYYMDVWGQGTLVTVSS |
| 9-8 | 542 | QVQLVESGGGVVQPGRSLRLSCAASGFTFSSYAMHWVRQAPGKGL |
| | | EWVAVISYDGNHEYYADSVKGRFTISRDNSKNTLYLQMNSLRAED |
| | | TAVYYCARGAKYYYMDVWGQGTLVTVSS |
| 9-9 | 543 | QVQLVESGGGVVQPGRSLRLSCAASGFIFRSYAMHWVRQAPGKGL |
| | | EWVAVISYDGSSKYYADSVKGRFTISRDNSKNTLYLQMNSLRAEDT |
| | | AVYYCARPSSGSYFPPFDYWGQGTLVTVSS |
| 9-10 | 544 | QVQLVESGGGVVQPGRSLRLSCAASGFTFSDYGMHWVRQAPGKGL |
| | | EWVAVVSYDGTTKYYADSVKGRFTISRDNSKNTLYLQMNSLRAED |
| | | TAVYYCAKENWLGYFDPWGQGTLVTVSS |
| 9-11 | 545 | QVQLVESGGGVVQPGRSLRLSCAASGFTFSNFPMHWVRQAPGKGL |
| | | EWVAVISYDGSLKYYADSVKGRFTISRDNSKNTLYLQMNSLRAEDT |
| | | AVYYCARYQGGYMDVWGQGTLVTVSS |
| 9-12 | 546 | QVQLVESGGGVVQPGRSLRLSCAASGPTFSRFAMHWVRQAPGKGL |
| | | EWVAVISYDGSNKYYADSVKGRFTISRDNSKNTLYLQMNSLRAED |
| | | TAVYYCARDTGLGFDPWGQGTLVTVSS |
| 9-13 | 547 | QVQLVESGGGVVQPGRSLRLSCAASGFTFNNYAMHWVRQAPGKG |
| | | LEWVAVISYDGNNKYYADSVKGRFTISRDNSKNTLYLQMNSLRAE |
| | | DTAVYYCAKTMGGSYFDAFDIWGQGTLVTVSS |
| 9-14 | 548 | QVQLVESGGGVVQPGRSLRLSCAASGFTFSDYTMHWVRQAPGKGL |
| | | EWVAVISYEGSIKYYADSVKGRFTISRDNSKNTLYLQMNSLRAEDT |
| | | AVYYCARSSSGSYPSLVDYWGQGTLVTVSS |
| 9-15 | 549 | QVQLVESGGGVVQPGRSLRLSCAASGFSESSYAMHWVRQAPGKGL |
| | | EWVAVISFDGSNEYYADSVKGRFTISRDNSKNTLYLQMNSLRAEDT |
| | | AVYYCARDYWVDYFKPGGRGALLTTSS |
| 10-1 | 550 | QVQLVESGGGVVQPGRSLRLSCAASGFTESRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 10-2 | 551 | QVQLVESGGGVVQPGRSLRLSCAASGFTFSRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 10-3 | 552 | QVQLVESGGGVVQPGRSLRLSCAASGFTFSRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 10-4 | 553 | QVQLVESGGGVVQPGRSLRLSCAASGFTFSRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 10-5 | 554 | QVQLVESGGGVVQPGRSLRLSCAASGFTFSRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 10-6 | 555 | QVQLVESGGGVVQPGRSLRLSCAASGFTFSRYAMHWVRQAPGKGL |
| | | EWVAVISYDGTNEYYADSVKGRFTISRDNSKNTLYLQMNSLRAED |
| | | TAVYYCARDTGLGFDPWGQGTLVTVSS |
| 11-1 | 556 | QVQLVESGGGVVQPGRSLRLSCAASGFTFGSYGMHWVRQAPGKGL |
| | | EWVAVISYDGGDEYYADSVKGRFTISRDNSKNTLYLQMNSLRAED |
| | | TAVYYCARDISRYGYYGMDVWGQGTLVTVSS |
| 11-2 | 557 | QVQLVESGGGVVQPGRSLRLSCAASGFTFGTYAMHWVRQAPGKG |
| | | LEWVAVVSTEGGITYYADSVKGRFTISRDNSKNILYLQMNSLRAE |
| | | DTAVYYCAGSYGAYFDYWGQGTLVTVSS |
| 11-3 | 558 | QVQLVESGGGVVQPGRSLRLSCAASGFTFSNFAMHWVRQAPGKGL |
| | | EWVAVISYDGNHEYYADSVKGRFTISRDNSKNTLYLQMNSLRAED |
| | | TAVYYCAKTNSGSYGGMFDYWGQGTLVTVSS |
| 11-4 | 559 | QVQLVESGGGVVQPGRSLRLSCAASGFTFDNYAMHWVRQAPGKG |
| | | LEWVAVISDDGRNKYYADSVKGRFTISRDNSKNTLYLQMNSLRAE |
| | | DTAVYYCAKDNYYDSSGYYGGGMDVWGQGTLVTVSS |
| 11-5 | 560 | QVQLVESGGGVVQPGRSLRLSCAASGFTFSSFAMHWVRQAPGKGL |
| | | EWVAVISYDGSNKYYADSVKGRFTISRDNSKNTLYLQMNSLRAED |
| | | TAVYYCARSRSGSYSSYFDYWGQGTLVTVSS |
| 11-6 | 561 | QVQLVESGGGVVQPGRSLRLSCAASGFTFGTYAMHWVRQAPGKG |
| | | LEWVAVVSTEGGTTYYADSVKGRFTISRDNSKNTLYLQMNSLRAE |
| | | DTAVYYCAGEYYDSSGSSIDYWGQGTLVTVSS |
| 11-7 | 562 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCARAKGGGYRGATDIWGQGTLVTVSS |
| 11-8 | 563 | QVQLVESGGGVVQPGRSLRLSCAASGFTFSSYAMHWVRQAPGKGL |
| | | EWVAVISYDGSNTYYADSYRGRFTISRDNSKNTLYLQMNSLRAEDT |
| | | AVYYCARPRGGSYWTYFDYWGQGTLVTVSS |
| 11-9 | 564 | QVQLVESGGGVVQPGRSLRLSCAASGFTFGTYAMHWVRQAPGKG |
| | | LEWVAVVSTEGGTTYYADSVKGRFTISRDNSKNTLYLQMNSLRAE |
| | | DTAVYYCAGSYGAYFDYWGQGTLVTVSS |
| 11-10 | 565 | QVQLVESGGGVVQPGRSLRLSCAASGFIFNNYGMHWVRQAPGKGL |
| | | EWVAVISYDGSNIYYADSVKGRFTISRDNSKNTLYLQMNSLRAEDT |
| | | AVYYCARDYNDGIGSYEGAPDSWGQGTLVTVSS |
| 11-11 | 566 | QVQLVESGGGVVQPGRSLRLSCAASGFTFDNYAMHWVRQAPGKG |
| | | LEWVAVISYDGSNKYYADSVKGRFTISRDNSKNTLYLQMNSLRAE |
| | | DTAVYYCLREGILWDVWGQGTLVTVSS |
| 11-12 | 567 | QVQLVESGGGVVQPGRSLRLSCAASGFTFSSQAMHWVRQAPGKGL |
| | | EWVAVISYDGSNKYYADSVKGRFTISRDNSKNTLYLQMNSLRAED |
| | | TAVYYCAKTEGGTYGGAFDIWGQGTLVTVSS |
| 11-13 | 568 | QVQLVESGGGVVQPGRSLRLSCAASGFSFSSYGMHWVRQAPGKGL |
| | | EWVAVISYDGSDKYYADSVKGRFTISRDNSKNTLYLQMNSLRAED |
| | | TAVYYCAKDNYYDSSGYYGGGMDVWGQGTLVTVSS |
| 11-14 | 569 | QVQLVESGGGVVQPGRSLRLSCAASGFTFSSYSMHWVRQAPGKGL |
| | | EWVAVISYDGSHKYYADSVKGRFTISRDNSKNTLYLQMNSLRAED |
| | | TAVYYCARDGWGYFDYWGQGTLVTVSS |
| 11-15 | 570 | QVQLVESGGGVVQPGRSLRLSCAASGFIFSNYGMHWVRQAPGKGL |
| | | EWVAVISYDGSDKYYADSVKGRFTISRDNSKNTLYLQMNSLRAED |
| | | TAVYYCARDDYMYGFEHWGQGTLVTVSS |
| 11-16 | 571 | QVQLVESGGGVVQPGRSLRLSCAASGFTFSDHYMHWVRQAPGKGL |
| | | EWVAVISYDGSNEYYADSVKGRFTISRDNSKNTLYLQMNSLRAEDT |
| | | AVYYCAKDLGPAGVDYWGQGTLVTVSS |
| 11-17 | 572 | QVQLVESGGGVVQPGRSLRLSCAASGFIFSSYAMHWVRQAPGKGL |
| | | EWVAVISYDGSNKYYADSVKGRFTISRDNSKNTLYLQMNSLRAED |
| | | TAVYYCARSRSGSYSSWFDYWGQGTLVTVSS |
| 11-18 | 573 | QVQLVESGGGVVQPGRSLRLSCAASGFTFGTYAMHWVRQAPGKG |
| | | LEWVAVISYDGNNKYYADSVKGRFTISRDNSKNTLYLQMNSLRAE |
| | | DTAVYYCAKTGSGSYYSWFDYWGQGTLVTVSS |
| 11-19 | 574 | QVQLVESGGGVVQPGRSLRLSCAASGFTFSSYAMHWVRQAPGKGL |
| | | EWVAVISYDGTNDYYADSVKGRFTISRDNSKNTLYLQMNSLRAED |
| | | TAVYYCARTRGGSYFTPFDYWGQGTLVTVSS |
| 11-20 | 575 | QVQLVESGGGVVQPGRSLRLSCAASGFTFDDYAMHWVRQAPGKG |
| | | LEWVAVISYDGSNKYYADSVKGRFTISRDNSKNTLYLQMNSLRAE |
| | | DTAVYYCASPHSGSYWAAFDIWGQGTLVTVSS |
| 12-1 | 576 | QVQLVESGGGVVQPGRSLRLSCASGFTFSYYGMHWVRQAPGKGL |
| | | EWVAVTSYDGSNKYYADSVKGRFTISRDNSKNTLYLQMNSLRAED |
| | | TAVYYCARPQGGSYFAAFDIWGQGTLVTVSS |
| 12-2 | 577 | QVQLVESGGGVVQPGRSLRLSCAASGFIFRSYAMHWVRQAPGKGL |
| | | EWVAVISYDGSSKYYADSVKGRFTISRDNSKNTLYLQMNSLRAEDT |
| | | AVYYCARPSSGSYFPPFDYWGQGTLVTVSS |
| 12-3 | 578 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCAKTRTGSYFSAFDIWGQGTLVTVSS |
| 12-4 | 579 | QVQLVESGGGVVQPGRSLRLSCAASGFTFSYYGMHWVRQAPGKGL |
| | | EWVAVISYDGTNDYYADSVKGRFTISRDNSKNTLYLQMNSLRAED |
| | | TAVYYCAKPHSGSYRGYFDYWGQGTLVTVSS |
| 12-5 | 580 | QVQLVESGGGVVQPGRSLRLSCAASGFTFSYYGMHWVRQAPGKGL |
| | | EWVAVISYDGSNKYYSDSVKGRFTISRDNSKNTLYLQMNSLRAED |
| | | TAVYYCARPKSGSYATYFDYWGQGTLVTVSS |
| 12-6 | 581 | QVQLVESGGGVVQPGRSLRLSCAASGFIFRNYAMHWVRQAPGKGL |
| | | EWVAVISYDGSNKYYADSVKGRFTISRDNSKNTLYLQMNSLRAED |
| | | TAVYYCARPRGGSYHGAFDIWGQGTLVTVSS |
| 12-7 | 582 | QVQLVESGGGVVQPGRSLRLSCASGFTESYAMHWVRQAPGKGL |
| | | EWVAVISYDGTNEYYADSVKGRFTISRDNSKNTLYLQMNSLRAED |
| | | TAVYYCAKSRGGSYYGAFDYWGQGTLVTVSS |
| 12-8 | 583 | QVQLVESGGGVVQPGRSLRLSCAASGFTFNNYVMHWVRQAPGKG |
| | | LEWVAVISYDGTNDYYADSVKGRFTISRDNSKNTLYLQMNSLRAE |
| | | DTAVYYCARGESGSYWGAFDYWGQGTLVTVSS |
| 12-9 | 584 | QVQLVESGGGVVQPGRSLRLSCAASGFTFSSYGMHWVRQAPGKGL |
| | | EWVAVISYDGTTEYYADSVKGRFTISRDNSKNTLYLQMNSLRAEDT |
| | | AVYYCARPSSGSYLGFFDYWGQGTLVTVSS |
| 12-10 | 585 | QVQLVESGGGVVQPGRSLRLSCAASGFIFRSYAMHWVRQAPGKGL |
| | | EWVAVISYDGSIKYYADSVKGRFTISRDNSKNTLYLQMNSLRAEDT |
| | | AVYYCARTRGGSYYGAFDYWGQGTLVTVSS |
| 12-11 | 586 | QVQLVESGGGVVQPGRSLRLSCAASGFSFGGYGMHWVRQAPGKG |
| | | LEWVAVISYDGSNEYYADSVKGRFTISRDNSKNTLYLQMNSLRAED |
| | | TAVYYCAKSYSGSYSSYFDYWGQGTLVTVSS |
| 12-12 | 587 | QVQLVESGGGVVQPGRSLRLSCAASGFAFSSHAMHWVRQAPGKGL |
| | | EWVAVISYDGSNKYYADSEKGRFTISRDNSKNTLYLQMNSLRAEDT |
| | | AVYYCAKAYSGSYMGYFDYWGQGTLVTVSS |
| 12-13 | 588 | QVQLVESGGGVVQPGRSLRLSCAASGPSPSTYGMHWVRQAPGKGL |
| | | EWVAVISYDGSNKYYADSVKGRFTISRDNSKNTLYLQMNSLRAED |
| | | TAVYYCARPLSGSYWSWFDPWGQGTLVTVSS |
| 12-14 | 589 | QVQLVESGGGVVQPGRSLRLSCAASGFTFSSYSMHWVRQAPGKGL |
| | | EWVAVISYDGSNKYYADSVKGRFTISRDNSKNTLYLQMNSLRAED |
| | | TAVYYCARGKGGGYYSSFDFWGQGTLVTVSS |
| 12-15 | 590 | QVQLVESGGGVVQPGRSLRLSCAASGFSFGGYGMHWVRQAPGKG |
| | | LEWVAVISYDGSNKYYADSVKGRFTISRDNSKNTLYLQMNSLRAE |
| | | DTAVYYCARPYSGSYISWFDYWGQGTLVTVSS |
| 12-16 | 591 | QVQLVESGGGVVQPGRSLRLSCAASGFIFRSYAMHWVRQAPGKGL |
| | | EWVAVISYDGSSKYYADSVKGRFTISRDNSKNTLYLQMNSLRAEDT |
| | | AVYYCARTLGGSYFAAFDIWGQGTLVTVSS |
| 12-17 | 592 | QVQLVESGGGVVQPGRSLRLSCAASGFTFGSYGMHWVRQAPGKGL |
| | | EWVAVISYDGNHEYYADSVKGRFTISRDNSKNTLYLQMNSLRAED |
| | | TAVYYCARPHSGSYTAYFDYWGQGTLVTVSS |
| 12-18 | 593 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCARGYGGSYSYFDYWGQGTLVTVSS |
| 12-19 | 594 | QVQLVESGGGVVQPGRSLRLSCAASGFAFSSYAMHWVRQAPGKGL |
| | | EWVAVISYDGTYEYYADSVKGRFTISRDNSKNTLYLQMNSLRAED |
| | | TAVYYCARSLGGSYFSGMDVWGQGTLVTVSS |
| 12-20 | 595 | QVQLVESGGGVVQPGRSLRLSCAASGFSFGGYGMHWVRQAPGKG |
| | | LEWVAVISYDGSNKYYADSVKGRFTISRDNSKNTLYLQMNSLRAE |
| | | DTAVYYCARSKGGSYYGPFDYWGQGTLVTVSS |
| 12-21 | 596 | QVQLVESGGGVVQPGRSLRLSCAASGFSFGGYGMHWVRQAPGKG |
| | | LEWVAVISYDGSNKYYADSVKGRFTISRDNSKNTLYLQMNSLRAE |
| | | DTAVYYCARPKGGNYWNAFDIWGQGTLVTVSS |
| 12-22 | 597 | QVQLVESGGGVVQPGRSLRLSCAASGFTFSSYGMHWVRQAPGKGL |
| | | EWVAVISYDGNHEYYADSVKGRFTISRDNSKNTLYLQMNSLRAED |
| | | TAVYYCARPKSGSYVSYFDYWGQGTLVTVSS |
| 12-23 | 598 | QVQLVESGGGVVQPGRSLRLSCAASGFIFSSYAMHWVRQAPGKGL |
| | | EWVAVISYDGSNKYYADSVKGRFTISRDNSKNTLYLQMNSLRAED |
| | | TAVYYCARPRGGNYLNYFDYWGQGTLVTVSS |
| 12-24 | 599 | QVQLVESGGGVVQPGRSLRLSCAASGFTFSNFPMHWVRQAPGKGL |
| | | EWVAVISYDGNNKYYADSVKGRFTISRDNSKNTLYLQMNSLRAED |
| | | TAVYYCAKDHGDHYFDYWGQGTLVTVSS |
| 12-25 | 600 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCARDKGGSYYGPFDYWGQGTLVTVSS |
| 12-26 | 601 | QVQLVESGGGVVQPGRSLRLSCAASGPTFSNYAMHWVRQAPGKGL |
| | | EWVAVISYDGSNEYYADSEKGRFTISRDNSKNTLYLQMNSLRAEDT |
| | | AVYYCAKSGSGSYFSPFDYWGQGTLVTVSS |
| 12-27 | 602 | QVQLVESGGGVVQPGRSLRLSCAASGFSFGGYGMHWVRQAPGKG |
| | | LEWVAVISYDGSTKYYADSVKGRFTISRDNSKNTLYLQMNSLRAED |
| | | TAVYYCARPRGGSYKDAFDIWGQGTLVTVSS |
| 12-28 | 603 | QVQLVESGGGVVQPGRSLRLSCAASGFTFSSYAMHWVRQAPGKGL |
| | | EWVAVISYDGTNEYYADSEKGRFTISRDNSKNTLYLQMNSLRAEDT |
| | | AVYYCARAHGGSYFSGMDVWGQGTLVTVSS |
| 12-29 | 604 | QVQLVESGGGVVQPGRSLRLSCAASGFSFSNYGMHWVRQAPGKGL |
| | | EWVAVISYDGNNKYYADSVKGRFTISRDNSKNTLYLQMNSLRAED |
| | | TAVYYCARSKGGSYYGPFDDWGQGTLVTVSS |
| 12-39 | 605 | QVQLVESGGGVVQPGRSLRLSCAASGFTFSGYAMHWVRQAPGKGL |
| | | EWVAVISYDGSNKWADSVKGRFTISRDNSKNTLYLQMNSLRAED |
| | | TAVYYCARSRGGSYYAPFDYWGQGTLVTVSS |
| 12-31 | 606 | QVQLVESGGGVVQPGRSLRLSCAASGFTFSYYTMHWVRQAPGKGL |
| | | EWVAVTSYDGSNKYYADSVKGRFTISRDNSKNTLYLQMNSLRAED |
| | | TAVYYCARPLGGSYFAAFDIWGQGTLVTVSS |
| 12-32 | 607 | QVQLVESGGGVVQPGRSLRLSCAASGPTFGTYAMHWVRQAPGKG |
| | | LEWVAVISYDGNNKYYADSVKGRFTISRDNSKNTLYLQMNSLRAE |
| | | DTAVYYCAKTMSGSYFSAFDIWGQGTLVTVSS |
| 12-33 | 608 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCARPHGGNYFDWFDPWGQGTLVTVSS |
| 12-34 | 609 | QVQLVESGGGVVQPGRSLRLSCAASGFIFRSYAMHWVRQAPGKGL |
| | | EWVAVISYDGSSKYYADSVKGRFTISRDNSKNTLYLQMNSLRAEDT |
| | | AVYYCARPSGGSYFDPFDYWGQGTLVTVSS |
| 12-35 | 610 | QVQLVESGGGVVQPGRSLRLSCAASGFTFSSSSMHWVRQAPGKGL |
| | | EWVAVISYDGSNKYYADSVKGRFTISRDNSKNTLYLQMNSLRAED |
| | | TAVYYCAKVDSGSYVGYFDYWGQGTLVTVSS |
| 12-36 | 611 | QVQLVESGGGVVQPGRSLRLSCAASGFSFNNYGMHWVRQAPGKG |
| | | LEWVAVISYDGSNDYYADSVKGRFTISRDNSKNTLYLQMNSLRAE |
| | | DTAVYYCARPNSGSYSNYFDYWGQGTLVTVSS |
| 12-37 | 612 | QVQLVESGGGVVQPGRSLRFSCAGTGFTFSSYAMHWVRQAPGKGL |
| | | EWVAVISYDGSNQYYADSVKGRFTISRDNSKNTLYLQMNSLRAED |
| | | TAVYYCARSRSGSYLAYFDYWGQGTLVTVSS |
| 12-38 | 613 | QVQLVESGGGVVQPGRSLRLSCAASGFTFSSYAMHWVRQAPGKGL |
| | | EWVAVISYDGSNQYYADSVKGRFTISRDNSKNTLYLQMNSLRAED |
| | | TAVYYCARAAGGSYSSWFDPWGQGTLVTVSS |
| 12-39 | 614 | QVQLVESGGGVVQPGRSLRLSCAASGFTFSSYAMHWVRQAPGKGL |
| | | EWVAVISYDGNHEYYADSVKGRFTISRDNSKNTLYLQMNSLRAED |
| | | TAVYYCARAHSGSYFSHFDYWGQGTLVTVSS |
| 12-40 | 615 | QVQLVESGGGVVQPGRSLRLSCAASGFTFSSYAMHWVRQAPGKGL |
| | | EWVAVISYDGSNTYYADSVKGRFTISRDNSKNTLYLQMNSLRAEDT |
| | | AVYYCARPTSGSYFSWFDPWGQGTLVTVSS |
| 12-41 | 616 | QVQLVESGGGVVQPGRSLRLSCAASGFIFSSYAMHWVRQAPGKGL |
| | | EWVAVISYDGSNKYYADSVKGRFTISRDNSKNTLYLQMNSLRAED |
| | | TAVYYCARPNSGSYWGPFDYWGQGTLVTVSS |
| 12-42 | 617 | QVQLVESGGGVVQPGRSLRLSCAASGFTFGSYGMHWVRQAPGKGL |
| | | EWVAVISYDGSHKYYADSVKGRFTISRDNSKNTLYLQMNSLRAED |
| | | TAVYYCARALGGNYYYFDYWGQGTLVTVSS |
| 12-43 | 618 | QVQLVESGGGVVQPGRSLRLSCAASGFIFSSYGMHWVRQAPGKGL |
| | | EWVAVISYDGSNEYYADSVKGRFTISRDNSKNTLYLQMNSLRAEDT |
| | | AVYYCARPRSGSYLSAFDYWGQGTLVTVSS |
| 13-1 | 619 | QVQLVESGGGVVQPGRSLRLSCAASGFTFSSYSMHWVRQAPGKGL |
| | | EWVAVISYDGRNQYYADSVKGRFTISRDNSKNTLYLQMNSLRAED |
| | | TAVYYCAKGYGGNYYYMDGWGQGTLVTVSS |
| 13-2 | 620 | QVQLVESGGGVVQPGRSLRLSCAASGFTFSSYAMHWVRQAPGKGL |
| | | EWVAVISYDGNNKYYADSVKGRFTISRDNSKNTLYLQMNSLRAED |
| | | TAVYYCARTYGGSYYSAFDYWGQGTLVTVSS |
| 13-3 | 621 | QVQLVESGGGVVQPGRSLRLSCAASGFSFNNHAMHWVRQAPGKG |
| | | LEWVAVISYDGSDKYYADSVKGRFTISRDNSKNTLYLQMNSLRAE |
| | | DTAVYYCARNLLRGYGMDVWGQGTLVTVSS |
| 13-4 | 622 | QVQLVESGGGVVQPGRSLRLSCAASGFAFDDYAMHWVRQAPGKG |
| | | LEWVAVISYDGSNKYYADSVKGRFTISRDNSKNTLYLQMNSLRAR |
| | | DTAVYYCATLGYGDYPDYWGQGTLVTVSS |
| 13-5 | 623 | QVQLVESGGGVVQPGRSLRLSCAASGFIFRSYAMHWVRQAPGKGL |
| | | EWVAVISYDGSSKYYADSVKGRFTISRDNSKNTLYLQMNSLRAEDT |
| | | AVYYCARPLGGGYQDAPDIWGQGTLVTVSS |

TABLE 13
Variable Domain Light Chain Sequences

| | SEQ ID | |
|---|---|---|
| Antibody | NO | VL |
| 9-1 | 624 | EIVLTQSPATLSLSPGERATLSCRASQGVSNYLAWYQQKPGQAPRL |
| | | LIYDASNRATGIPARFSGSGSGTDFTLTISSLEPEDFAVYYCQQRYSW |
| | | VTFGGGTKVEIK |
| 9-2 | 625 | EIVLTQSPATLSLSPGERATLSCRASQSVSSSLAWYQQKPGQAPRLLL |
| | | YDASNRATGIPARFSGSGSGTDFTLTISSLEPEDFAVYYCQQRINWP |
| | | RSFGGGTKVEIK |
| 9-3 | 626 | EIVLTQSPATLSLSPGERATLSCRASQSVNSYLAWYQQKPGQAPRLL |
| | | IYDVSNRATGIPARFSGSGSGTDFTLTISSLEPEDFAVYYCQQFSNWP |
| | | TFGGGTKVEIK |
| 9-4 | 627 | EIVLTQSPATLSLSPGERATLSCRASQSVGTSLAWYQQKPCQAPRLL |
| | | IYGASNRATGIPARFSGSGSGTDFTLTISSLEPEDFAVYYCQQRSNW |
| | | QPFGGGTKVEIK |
| 9-5 | 628 | EIVLTQSPATLSLSPGERATLSCRATQYVNSYLAWYQQKPRQAPRLI |
| | | IYDVSNRATGIPARFSGSGSGTDFTLTISSLEPEDFAVYYCQQFSNWP |
| | | TFGGGTKVEIK |
| 9-6 | 629 | EIVLTQSPATLSLSPGERATLSCRASQSVGTSLAWYQQKPGQAPRLL |
| | | IYGASNRATGIPARFSGSGSGTDFTLTISSLEPEDFAVYYCQLRSNWY |
| | | TFGGGTKVEIK |
| 9-7 | 630 | EIVLTQSPATLSLSPGERATLSCRASQGVSNYLAWYQQKPGQAPRL |
| | | LIYDASNRATGIPARFSGSGSGTDFILSISSLEPEDFAVYYCQQRYSW |
| | | VTFGGGTKVEIK |
| 9-8 | 631 | EIVLTQSPATLSLSPGERATLSCRASQGVSNYLAWYQQKPGQAPRL |
| | | LIYDASNRATGIPARFSGSGSGTDFTLTISSLEPEDFAVYYCQQRYSW |
| | | VTFGGGTKVEIK |
| 9-9 | 632 | EIVLTQSPATLSLSPGERATLSCRASQSVDSRLAWYQQKPGQAPRLL |
| | | IYDTSNRATGIPARFSGSGSGTDFTLTISSLEPEDFAVYYCQQRSTWP |
| | | PVFGGGTKVEIK |
| 9-10 | 633 | EIVLTQSPATLSLSPGERATLSCRASQSVRHHLAWYQQKPGQAPRLL |
| | | IYDASNRATGIPARFSGSGSGTDFTLTISSLEPEDFAVYYCQQRTDWP |
| | | RAPGGGTKVEIK |
| 9-11 | 634 | ETVLTQSPATLSLSPGERATLSCRASQSVGNFLAWYQQKPGQAPRLL |
| | | IYDASNRATGIPARFSGSGSGTDFTLTISSLEPEDFAVYYCQQSSTWP |
| | | LTFGGGTKVEIK |
| 9-12 | 635 | EIVLTQSPATLSLSPGERATLSCRASESISTYLAWYQQKPGQAPRLLI |
| | | YDASNRATGIPARFSGSGSGTDFTLTISSLEPEDFAVYYCQQRSGLIT |
| | | PGGGTKVEIK |
| 9-13 | 636 | ETVLTQSPATLSLSPGERATLSCRASQSVGDFLAWYQQKPGQAPRLL |
| | | IYDTSNRATGIPARFSGSGSGTDFTLTISSLEPEDFAVYYCQQRSNLT |
| | | FGGGTKVEIK |
| 9-14 | 637 | DIQMTQSPSSLSASVGDRVTITCRASQTIRNSLNWYQQKPGKAPKLL |
| | | IYASSSLQSGVPSRFSGSGSGTDFTLTISSLQPEDFATYYCQQTHSIPK |
| | | TFGQGTKVEIK |
| 9-15 | 638 | EIVLTQSPATLSLSPGERATLSCRASQSVSSSLAWYQQKPGQAPRLLI |
| | | YDASNRATGIPARFSGSGSGTDFTLTISSLEPEDFAVYYCQQRINWP |
| | | RSPGGGTKVEIK |
| 10-1 | 639 | EIVLTQSPATLSLSPGERATLSCRASQDVSTYLAWYQQKPGQAPRLL |
| | | IYDASNRATGIPARFSGSGSGTDFTLTISSLEPEDFAVYYCQQRRDW |
| | | PQTFGGGTKVEIK |
| 10-2 | 640 | EIVLTQSPATLSLSPGERATLSCRASQDVSTYLAWYQQKPGQAPRLL |
| | | IYDASNRATGIPARFSGSGSGTDFTPFITNFEPEDPAVYYCQQRRDW |
| | | PQTFGGGTKVEIK |
| 10-3 | 641 | ETVLTQSPATLSLSPGERATLSCRASQDVSTYLAWYQQKPGQAPRLL |
| | | IYDASNRATGIPARFSGSGSGTDFTPFITNFEPEDFAVYYCQQRRDW |
| | | PQTFGGGTKVEIK |
| 10-4 | 642 | EIVLTQSPATLSLSPGERATLSCRASQDVSTYLAWYQQKPGQAPRLL |
| | | IYDASNRATGIPARFSGSGSGTDFTLFITNFEPEDFAVYYCQQRRDW |
| | | PQTFGGGTKVEIK |
| 10-5 | 645 | EIVLTQSPATLSLSPGERATLSCRASQDVSTYLAWYQQKPGQAPRLL |
| | | IYDASNRATGIPARFSGSGSCTDFTLTISSFEPEDFAVYYCQQRRDWP |
| | | QTFGGGTKVEIK |
| 10-6 | 644 | EIVLTQSPATLSLSPGERATLSCRASQDVSTYLAWYQQKPGQAPRLL |
| | | IYDASNRATGIPARFSGSGSGTDFTPFITNFEHEDFAVYYCQQRRDW |
| | | PQTFGGGTKVEIK |
| 11-1 | 645 | EIVLTQSPATLSLSPGERATLSCRASQSLGSFLAWYQQKPGQAPRLLL |
| | | YDASNRATGIPARFSGSGSGTDFTLTISSLEPEDFAVYYCQQRALWP |
| | | RLTPGGGTKVEIK |
| 11-2 | 646 | EIVLTQSPATLSLSPGERATLSCRASQSVNSYLAWYQQKPGQAPRLL |
| | | IYDVSNRATGIPARFSGSGSGTDFTLTISSLEPEDFAVYYCQQFSNWP |
| | | TFGGGTKVEIK |
| 11-3 | 647 | EIVLTQSPATLSLSPGERATLSCRASQNIGNHLAWYQQKPGQAPRLL |
| | | IYDASNRATGIPARFSGSGSGTDFTLTISSLEPEDFAVYYCQQRDNGP |
| | | PEGTFGQGTKVEIK |
| 11-4 | 648 | EIVLTQSPATLSLSPGERATLSCRASQSVGSYLAWYQQKPGQAPRLL |
| | | IYDAVNRATGIPARFSGSGSGTDFTLTISSLEPEDFAVYYCQQRFTWP |
| | | TTFGGGTKVEIK |
| 11-5 | 649 | EIVLTQSPATLSLSPGERATLSCRASQSITDYLAWYQQKPGQAPRLLI |
| | | YDASNRATGIPARFSGSGSGTDFTLTISSLEPEDEAVYYCHQRNNWP |
| | | PTFGGGTKVEIK |
| 11-6 | 650 | EIVLTQSPATLSLSPGERATLSCRASQSVDSSLAWYQQKPGQAPRLL |
| | | IYDASNRATGIPARFSGSGSGTDFTLTISSLEPEDFAVYYCQQQSNWP |
| | | GTFGGGTKVEIK |
| 11-7 | 651 | ETVLTQSPATLSLSPGERATLSCRASQSIGSYLAWYQQKPGQAPRLLI |
| | | YDGSNRATGIPARFSGSGSGTDFTLTISSLEPEDFAVYYCQQRTNWP |
| | | LFSFGGGTKVEIK |
| 11-8 | 652 | EIVLTQSPATLSLSPGERATLSCRASQTVTNYLAWYQQKPGQAPRLL |
| | | IYDTSNRATGIPARFSGSGSGTDFTLTISSLEPEDFAVYYCQHRDDWP |
| | | PTFGGGTKVEIK |
| 11-9 | 653 | EIVLTQSPATLSLSPGERATLSCRASQSVSYYLAWYQQKPGQAPRLL |
| | | IYDSSNRATGIPARFSGSGSGTDFTLTISSLEPEDFAVYYCQQRSNWQ |
| | | GNFGGGTKVEIK |
| 11-10 | 654 | EIVLTQSPATLSLSPGERATLSCRASQSVSTSLAWYQQKPGQAPRLLI |
| | | YDATNRATGIPARFSGSGSGTDFTLTISSLEPEDFAVYYCQQHYSWP |
| | | LTFGGGTKVEIK |
| 11-11 | 655 | EIVLTQSPATLSLSPGERATLSCRASHNINNFLAWYQQKPGQAPRLLI |
| | | YDTSNRATGIPARFSGSGSGTDFTLTISSLEPEDFAVYYCQQGRNWP |
| | | PSSFGGGTKVEIK |
| 11-12 | 656 | EIVLTQSPATLSLSPGERATLSCRASQSVGTSLAWYQQKPGQAPRLL |
| | | IYGASNRATGIPARFSGSGSGTDFTLTISSLEPEDFAVYYCQERSNWP |
| | | DTFGGGTKVEIK |
| 11-13 | 657 | EIVLTQSPATLSLSPGERATLSCRASQSVSSQLAWYQQKPGQAPRLL |
| | | MYDTSNRATGIPARFSGSGSGTDFTLTISSLEPEDPAVYYCQQRYNW |
| | | PSTFGGGTKVEIK |
| 11-14 | 658 | EIVLTQSPATLSLSPGERATLSCRASQSVDSRLAWYQQKPGQAPRLL |
| | | IYDASNRATGIPARFSGSGSGTDFTLTISSLEPEDFAVYYCQQRTNLP |
| | | PSITFGGGTKAKLK |
| 11-15 | 659 | EMVVPQSPPTVSLSPGERATLSCRASQSVGSYLAWYQQKPGQAPRL |
| | | LIYDAVNRATGIPARFSGSGSGTDFTLTISSLEPEDFAVYYCQQRSDS |
| | | ITFGGGTKVEIK |
| 11-16 | 660 | EIVLTQSPATLSLSPGERATLSCRASQSLGRYLAWYQQKPGQAPRLL |
| | | IYDSSNRATGIPARFSGSGSGTDFTLTISSLEPEDPAVYYCQQYGDWP |
| | | ETFGGGTKVEIK |
| 11-17 | 661 | EIVLTQSPATLSLSPGERATLSCRASQNIGSHLAWYQQKPGQAPRLLI |
| | | YDVSNRATGIPARFSGSGSGTDFTLTISSLEPEDFAVYYCQQRDYWP |
| | | PYTFGGGTKVEIK |
| 11-18 | 662 | EIVLTQSPATLSLSPGERATLSCRASQSLTSYLAWYQQKPGQAPRLLI |
| | | YDASNRATGIPARFSGSGSGTDFTLTISSLEPEDFAVYYCQQRHYWP |
| | | PITFGGGTKVEIK |
| 11-19 | 663 | EIVLTQSPATLSLSPGERATLSCRASQSIGSYLAWYQQKPGQAPRLLI |
| | | YDASNRATGIPARFSGSGSGTDFTLTISSLEPEDFAVYYCQQRDSWP |
| | | HTFGGGTKVEIK |
| 11-20 | 664 | EIVLTQSPATLSLSPGERATLSCRASQSVGSYLAWYQQKPGQAPRLL |
| | | IYDAVNRATGIPARFSGSGSGTDFTLTISSLEPEDRAVYYCQQRSLWP |
| | | FGGGTKVELK |
| 12-1 | 665 | EIVLTQSPATLSLSPGERATLSCRASQSVSSHLAWYQQKPGQAPRLL |
| | | IYDVSNRATGIPARFSGSGSGTDFTLTISSLEPEDFAVYYCQQRDTFT |
| | | FGGGTKVEIK |
| 12-2 | 666 | EIVLTQSPATLSLSPGERATLSCRASQSVDSRLAWYQQKPGQAPRLL |
| | | IYDTSNRATGIPARFSGSGSGTDFTLTISSLEPEDFAVYYCQQRSTWP |
| | | PVFGGGTKVEIK |
| 12-3 | 667 | EIVLTQSPATLSLSPGERATLSCRASQSVGDFLAWYQQKPGQAPRLL |
| | | IYDTSNRATGIPARFSGSGSGTDFTLTISSLEPEDPAVYYCQYRSNFT |
| | | FGGGTKVEIK |
| 12-4 | 668 | EIVLTQSPATLSLSPGERATLSCRASQSVGSHLAWYQQKPGQAPRLL |
| | | IYDASNRATGIPARFSGSGSGTDFTLTISSLEPEDFAVYYCQQISNWP |
| | | LTFGGGTKVEIK |
| 12-5 | 669 | ETVLTQSPATLSLSPGERATLSCRASQNVGQSLAWYQQKPGQAPRL |
| | | LIYDASNRATGIPARFSGSGSGTDFTLTISSLEPEDFAVYYCQQREN |
| | | WPPTFGGGTKVEIK |
| 12-6 | 670 | EIVLTQSPATLSLSPGERATLSCRASQSLGNYLAWYQQKPGQAPRLL |
| | | IYDSSNRATGIPARFSGSGSGTDFTLTISSLEPEDFAVYYCQQRNWPY |
| | | TFGGGTKVEIK |
| 12-7 | 671 | EIVLTQSPATLSLSPGERATLSCRASQSLGNYLAWYQQKPGQAPRLL |
| | | IYDSSNRATGIPARFSGSGSGTDFTLTISSLEPEDEAVYYCQQRTDWP |
| | | PSFGGGTKVEIK |
| 12-8 | 672 | EIVLTQSPATLSLSPGERATLSCRASQNIGNHLAWYQQKPGQAPRLL |
| | | IYDVSNRATGIPARFSGSGSGTDFTLTISSLEPEDFAVYYCQQRKSWP |
| | | PFTFGGGTKVEIK |
| 12-9 | 673 | EIVLTQSPATLSLSPGERATLSCRASQSVSTSLAWYQQKPGQAPRLLI |
| | | YDATNRATGIPARFSGSGSGTDFTLTISSLEPEDFAVYYCQRRTDWP |
| | | PTFGGGTKVEIK |
| 12-10 | 674 | ETVLTQSPATLSLSPGERATLSCRASQSVNSDLAWYQQKPGQAPRLL |
| | | IYDASNRATGIPARFSGSGSGTDFTLTISSLEPEDFAVYYCQQRTDWP |
| | | PATFGGGTKVEIK |
| 12-11 | 675 | ETVLTQSPATLSLSPGERATLSCRASQSVGSYLAWYQQKPGQAPRLL |
| | | IYDAVNRATGIPARFSGSGSGTDFTLTISSLEPEDFAVYYCQQRFTWP |
| | | TTFGGGTKVEIK |
| 12-12 | 676 | EIVLTQSPATLSLSPGERATLSCRASQSVSSSLAWYQQKPGQAPRLLI |
| | | YDASNRATGIPARFSGSGSGTDFTLTISSLEPEDFAVYYCQHRDDWP |
| | | PTFGGGTKVEIK |
| 12-13 | 677 | EIVLTQSPATLSLSPGERATLSCRASQSVGSYLAWYQQKPGQAPRLL |
| | | IYDAVNRATGIPARFSGSGSGTDFTLTISSLEPEDFAVYYCQQRNSW |
| | | PPATFGGGTKVEIK |
| 12-14 | 678 | EIVLTQSPATLSLSPGERATLSCRASQSVGSYLAWYQQKPGQAPRLL |
| | | IYDAVNRATGIPARFSGSGSGTDFTLTISSLEPEDFAVYYCQQVSNW |
| | | PLTPGGGTKVEIK |
| 12-15 | 679 | EIVLTQSPATLSLSPGERATLSCRASQSVSSHLAWYQQKPGQAPRLL |
| | | IYDVSNRATGIPARPSGSGSGTDFTLTISSLEPEDFAVYYCQVRSDWP |
| | | PLTFGGGTKVEIK |
| 12-16 | 680 | EIVLTQSPATLSLSPGERATLSCRASQSLDSYLAWYQQKPGQAPRLL |
| | | IYDVSNRATGIPARFSGSGSGTDFTLTISSLEPEDFAVYYCQQRRGW |
| | | PPVTFGGGTKVEIK |
| 12-17 | 681 | EIVLTQSPATLSLSPGERATLSCRASQSVSKFLAWYQQKPGQAPRLL |
| | | IYDASNRATGIPARFSGSGSGTDFTLTISSLEPEDFAVYYCHQHSDWP |
| | | LTFGGGTKVEIK |
| 12-18 | 682 | EIVLTQSPATLSLSPGERATLSCRASQSIGGSLAWYQQKPGQAPRLLI |
| | | YDASNRATGIPARFSGSGSGTDFTLTISSLEPEDFAVYYCQQRYSYFT |
| | | FGGGTKVEIK |
| 12-19 | 683 | EIVLTQSPATLSLSPGERATLSCRASQSISRYLAWYQQKPGQAPRLLI |
| | | YDVSNRATGIPARFSGSGSGTDFTLTISSLEPEDFAVYYCQQSSNWP |
| | | LFTFGGGTKVEIK |
| 12-20 | 684 | EIVLTQSPATLSLSPGERATLSCRASQSLGNYLAWYQQKPGQAPRLL |
| | | IYDSSNRATGIPARFSGSGSGTDFTLTISSLEPEDFAVYYCQQRNTWP |
| | | GVTFGGGTKVEIK |
| 12-21 | 685 | EIVLTQSPATLSLSPGERATLSCRASQSVNSDLAWYQQKPGQAPRLL |
| | | IYDASNRATGIPARFSGSGSGTDFTLTISSLEPEDFAVYYCQERSLFG |
| | | GGTKVEIK |
| 12-22 | 686 | EIVLTQSPATLSLSPGERATLSCRASQSVRHHLAWYQQKPGQAPRLL |
| | | IYDASNRATGIPARFSGSGSGTDFTLTISSLEPEDFAVYYCQERSDWP |
| | | ITFGGGTKVEIK |
| 12-23 | 687 | EIVLTQSPATLSLSPGERATLSCRASQSVDSRLAWYQQKPGQAPRLL |
| | | IYDASNRATGIPARFSGSGSGTDFTLTISSLEPEDFAVYYCQQRSTWP |
| | | PVFGGGTKVEIK |
| 12-24 | 688 | EIVLTQSPATLSLSPGERATLSCRASQSFGDSLAWYQQKPGQAPRLL |
| | | IYDASNRATGIPARFSGSGSGTDFTLTISSLEPEDFAVYYCQQRSIPIT |
| | | FGGGTKVEIK |
| 12-25 | 689 | ETVLTQSPATLSLSPGERATLSCRASQSVNSYLAWYQQKPGQAPRLL |
| | | IYDVSNRATGIPARFSGSGSGTDFTLTISSLEPEDFAVYYCQERGNWP |
| | | PFTFGGGTKVEIK |
| 12-26 | 690 | EIVLTQSPATLSLSPGERATLSCRASQSVSTSLAWYQQKPGQAPRLLI |
| | | YDISNRATGIPARFSGSGSGTDFTLTISSLEPEDFAVYYCQQRRSGLT |
| | | FGGGTKVEIK |
| 12-27 | 691 | EIVLTQSPATLSLSPGERATLSCRASDTVSSYLAWYQQKPGQAPRLL |
| | | IYDTSNRATGIPARFSGSGSGTDFTLTISSLEPEDFAVYYCQQRASWP |
| | | LSFGGGTKVEIK |
| 12-28 | 692 | EIVLTQSPATLSLSPGERATLSCRASQSVRHHLAWYQQKPGQAPRLL |
| | | IYDASNRATGIPARFSGSGSGTDFTLTISSLEPEDPAVYYCQQSGSWP |
| | | LTFGGGTKVEIK |
| 12-29 | 693 | EIVLTQSPATLSLSPGERATLSCRASQLISSYLAWYQQKPGQAPRLLI |
| | | YDTSNRATGIPARFSGSGSGTDFTLTISSLEPEDFAVYYCQVRSNWP |
| | | PLTFGGGTKVEIK |
| 12-30 | 694 | EIVLTQSPATLSLSPGERATLSCRASHNIGTYLAWYQQKPGQAPRLL |
| | | IYDVSNRATGIPARFSGSGSGTDFTLTISSLEPEDFAVYYCQQRADW |
| | | PQTFGGGTKVEIK |
| 12-31 | 695 | EIVLTQSPATLSLSPGERATLSCRASQSIGSYLAWYQQKPGQAPRLLI |
| | | YDVSNRATGIPARFSGSGSGTDFTLTISSLEPEDFAVYYCQQRDSFTE |
| | | GGGTKVEIK |
| 12-32 | 696 | ETVLTQSPATLSLSPGERATLSCRASQSIGSYLAWYQQKPGQAPRLLI |
| | | YDVSNRATGIPARFSGSGSGTDFTLTISSLEPEDFAVYYCQQRDSFTF |
| | | GGGTKVEIK |
| 12-33 | 697 | EIVLTQSPATLSLSPGERATLSCRASQDVSTYLAWYQQKPGQAPRLL |
| | | IYDASNRATGIPARFSGSGSGTDFTLTISSLEPEDFAVYYCQQRAYW |
| | | PGTFGGGTKVEIK |
| 12-34 | 698 | EIVLTQSPATLSLSPGERATLSCRASQSVGNFLAWYQQKPGQAPRLL |
| | | IYDASNRATGIPARFSGSGSCTDFTLTISSLEPEDFAVYYCQHRRLLT |
| | | FGGGTKVEIK |
| 12-35 | 699 | EIVLTQSPATLSLSPGERATLSCRASQRVSSYLAWYQQKPGQAPRLL |
| | | IYDAFNRATGIPARFSGSGSGTDFTLTISSLEPEDFAVYYCQQRQDW |
| | | PLTFGGGTKVEIK |
| 12-36 | 700 | EIVLTQSPATLSLSPGERATLSCRASQGISTYLAWYQQKPGQAPRLLI |
| | | YDASNRATGIPARFSGSGSGTDFTLTISSLEPEDFAVYYCQQRRRWP |
| | | PTFGGGTKVEIK |
| 12-37 | 701 | EIELTQSPATLSLSPGERATLSCRASESVSESLAWYQQKPGQAPRLLI |
| | | YDASNRATGIPARFSGSGSGTDFTLTISSLEPEDEAVYYCQQRTHGV |
| | | TFGGGTKVEIK |
| 12-38 | 702 | EIVLTQSPATLSLSPGERATLSCRASQSVSTSLAWYQQKPGQAPRLLI |
| | | YDATNRATGIPARFSGSGSGTDFTLTISSLEPEDFAVYYCQQRQKWP |
| | | LTFGGGTKVEIK |
| 12-39 | 703 | EIVLTQSPATLSLSPGERATLSCRASESISTYLAWYQQKPGQAPRLLI |
| | | YDASNRATGIPARFSGSGSGTDFTLTISSLEPEDLAVYYCQQRRNSL |
| | | TFGGGTKVEIK |
| 12-40 | 704 | EIVLTQSPATLSLSPGERATLSCRASQSVNSDLAWYQQKPGQAPRLL |
| | | IYDASNRATGIPARFSGSGSGTDFTLTISSLEPEDFAVYYCQQRSTWS |
| | | PLTFGGGTKVEIK |
| 12-41 | 705 | ETVLTQSPATLSLSPGERATLSCRASQNVGQSLAWYQQKPGQAPRL |
| | | LIYDASNRATGIPARFSGSGSGTDFTLTISSLEPEDFAVYYCQLRTNW |
| | | PPVTFGGGTKVEIK |
| 12-42 | 706 | EIVLTQSPATLSLSPGERATLSCRASQSVDSRLAWYQQKPGQAPRLL |
| | | IYDTSNRATGIPARFSGSGSGTDFTLTISSLEPEDPAVYYCQQRSSNW |
| | | TFGGGTKVEIK |
| 12-43 | 707 | EIVLTQSPATLSLSPGERATLSCRASQSVGKSLAWYQQKPGQAPRLL |
| | | IYDTSNRATGIPARFSGSGSGTDFTLTISSLEPEDFAVYYCQQRGSFP |
| | | LTFGGGTKVEIK |
| 13-1 | 708 | ETVLTQSPATLSLSPGERATLSCRASQSVGDFLAWYQQKPGQAPRLL |
| | | IYDTSNRATGIPARFSGSGSGTDFTLTISSLEPEDFAVYYCQQRSIRGT |
| | | FGGGTKVEIK |
| 13-2 | 709 | EIVLTQSPATLSLSPGERATLSCRASDTVSSYLAWYQQKPGQAPRLL |
| | | IYDTSNRATGIPARFSGSGSGTDFTLTISSLEPEDFAVYYCQQRGGWP |
| | | PAFGGGTKVEIK |
| 13-3 | 710 | DIQMTQSPSSLSASVGDRVTITCRASQSIGDYLNWYQQKPGKAPKL |
| | | LIYEASSLQSGVPSRFSGSGSGTDFTLTISSLQPEDFATYYCLHTYLPP |
| | | YSFGGGTKVEIK |
| 13-4 | 711 | DIQMTQSPSSLSASVGDRVTITCRASQSITRYLNWYQQKPGKAPKLL |
| | | IYAASSLQSGVPSRFSGSGSGTDFTLTISSLQPEDFATYYCQQTYNFP |
| | | HTFGQGTKVEIK |
| 13-5 | 712 | EIVLTQSPATLSLSPGERATLSCRASQSIGSYLAWYQQKPGQAPRLLI |
| | | YDVSNRATGIPARFSGSGSGTDFTLTISSPEPEDFAVYYCQQRHHWP |
| | | PVTFGGGTKVEIK |

### Example 7: Antibody 813

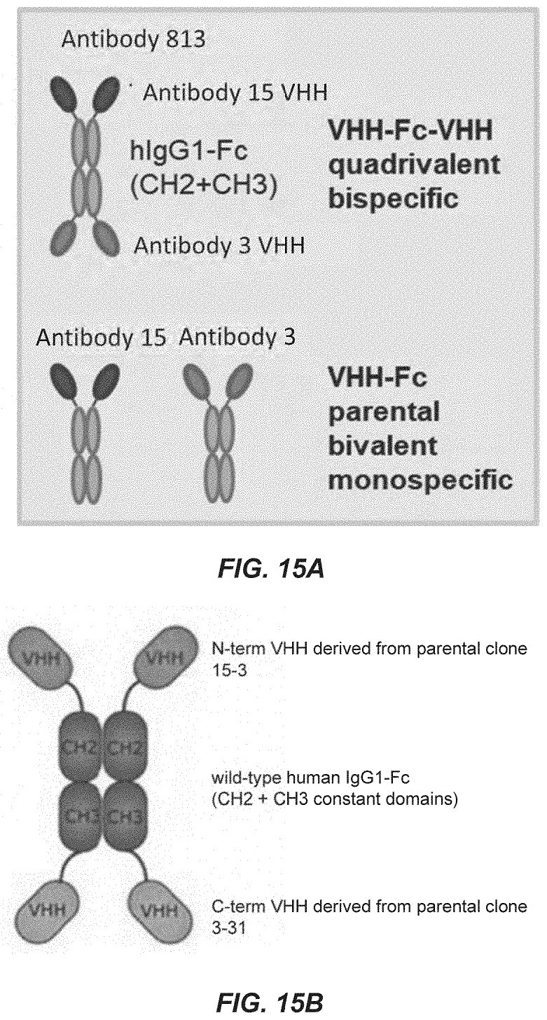

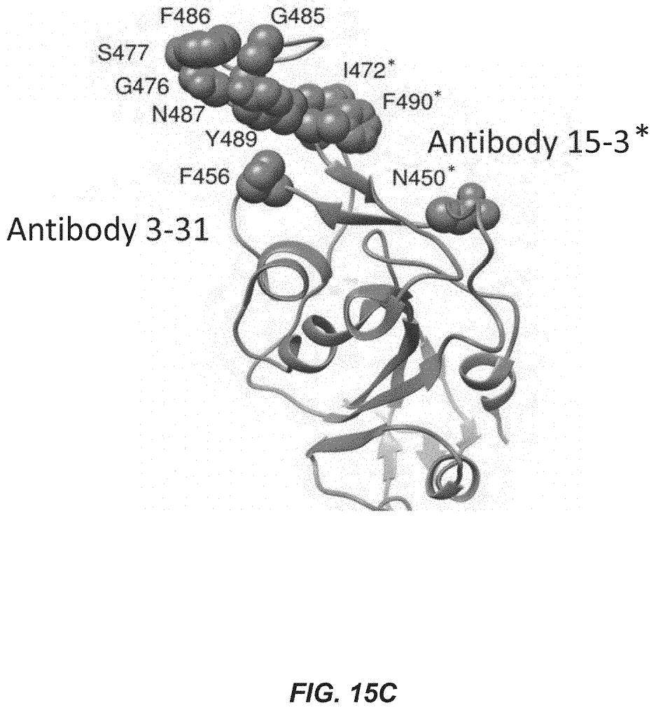

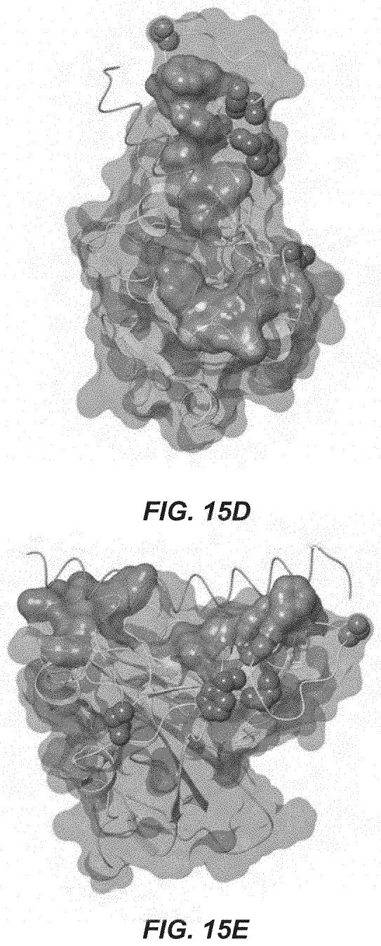

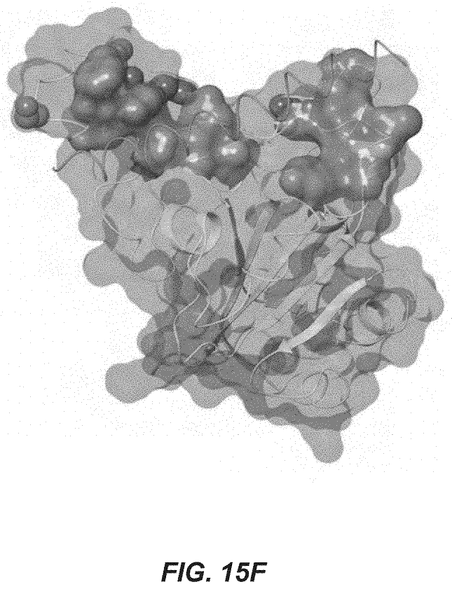

These experiments test antibody 813, a quadrivalent bispecific antibody made from parental bivalent monospecific antibodies 15 (specifically, 15-3) and 3 (specifically, 3-31) (FIGS. 15A-15B). The sequence for Antibody 813 can be found in Table 14.

TABLE 14
Antibody Sequences

| | SEQ ID | |
|---|---|---|
| Antibody | NO | Sequence |
| 813 | 713 | EVQLVESGGGLVQPGGSLRLSCAASGFTFSPSWMGWFRQAPGKER |
| | | EFVATINEYGGRNYADSVKGRFTISADNSKNTAYLQMNSLKPEDTA |
| | | VYYCARVDRDFDYWGQGTLVTVSSGGGGSEPKSSDKTHTCPPCPA |
| | | PELLGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKS |
| | | RWQQGNVFSCSVMHEALHNHYTQKSLSLSPGGGGGSGGGGSASEV |
| | | QLVESGGGLVQPGGSLRLSCAASGSTFSINAMGWFRQAPGKEREFV |
| | | AGITSSGGYTNYADSVKGRFTISADNSKNTAYLQMNSLKPEDTAVY |
| | | YCAADGVPEYSDYASGPVWGQGTLVTVSS |

Epitope alanine scanning mutagenesis experiments were performed to identify for each VHH binding specificity of the Antibody 813 bispecific using the VHH-Fc parents (Antibody 15-3 and Antibody 3-31) (FIG. 15C). In this experiment, a comprehensive SARS-CoV-2 (Wuhan-Hu-1) S protein RBD mutation library was generated in which all 184 residues (between residues 335-526) were individually mutated to alanine, and alanine residues to serine. Each mutation was confirmed by DNA sequencing, and clones were arrayed in a 384-well plate, one mutant per well. Cells expressing the SARS-CoV-2 RBD mutants were fixed, permeabilized, and immuno-stained with the indicated antibodies. Mean cellular fluorescence was detected by flow cytometry. A distinct set of critical contacts were identified for each VHH, with none shared. Critical contacts of parental Antibody 15-3, located at the N-terminus of Antibody 813, were identified as N450, I472, and F490. Critical contacts of parental Antibody 3-31, located at the C-terminus of Antibody 813, were identified as F456, G476, S477, G485, F486, N487, and Y489, with S477 and Y489 identified as likely most important. As depicted in FIG. 15C, VHH epitope footprints are so closely adjacent such that they exclude one another (e.g., the two VHHs cannot bind simultaneously to the same receptor binding domain (RBD)). Antibody 913's epitope was also investigated in the context of an ACE2 binding interface on the Ancestral SARS-CoV-2 RBD (FIGS. 15D-15F).

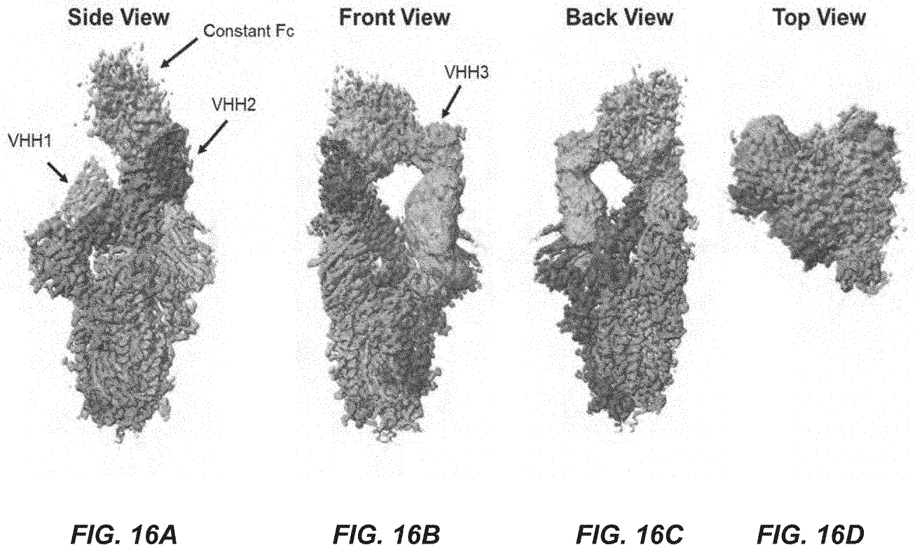

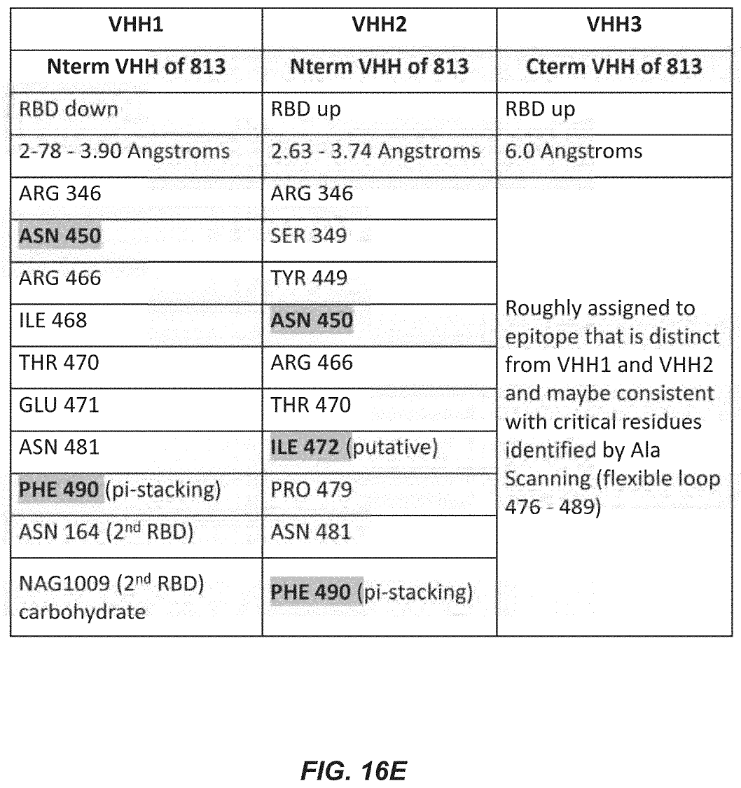

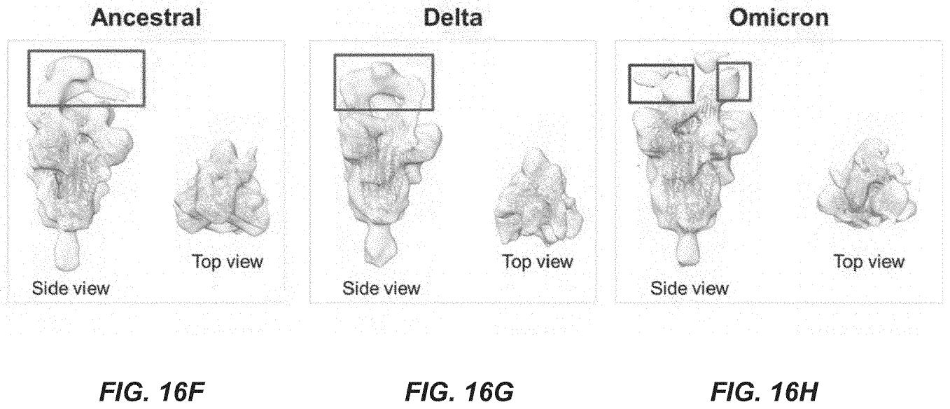

Critical contacts deduced here were confirmed by cryo-EM as seen in FIGS. 16A-E, which show reconstructed density m aps of the Antibody 813/Ancestral spike trimer complex viewed in different poses. FIG. 16E lists spike residues in explicit bonds for the three distinct binding epitopes (VHH1, VHH2, and VHH3) while highlighted residues represent the critical contacts which were also identified in the alanine scanning mutagenesis experiments. Antibody 813 engages all three RBDs in the spike trimer (two RBDs ‘up’ and one RBD ‘down’), demonstrating a multi-valent attachment. Densities were identified for three out of the four VHHs present on the bispecific Antibody 813 and the constant Fc fragment (with the 4th VHH location unconfirmed). Explicit bonds were identified for two of the three binding sites, representing the two identical N-term VHHs in Antibody 813, binding to RBD ‘down’ (VHH1) and ‘up’ (VHH2). VHH3 represents the C-term VHH in Antibody 813 and binds a distinct epitope (possibly a flexible loop) on a third RBD in the ‘up’ position (precise contacts unresolved). VHH1 and VHH2 epitopes confirmed the critical contacts identified by Alanine Scanning Mutagenesis (e.g., ASN 450, ILE 472 and PHE 490).

Negative stain analysis experiments show that Antibody 813 (bispecific) is bound to 1) the Ancestral variant and Delta spike trimers with two RBDs ‘up’ and 2) the Omicron spike trimer with only one RBD ‘up’. Multi-valent attachment of Antibody 813 is observed in all three complexes, consistent with avidity-boosted binding. Particles readily reconstruct into plausible 3D map at ˜14 Å resolution and agree with published coordinates for two RBDs ‘up’ (PBD ID 7DD2) for Ancestral and Delta, and one RBD ‘up’ (PBD ID 7V7P) for Omicron.

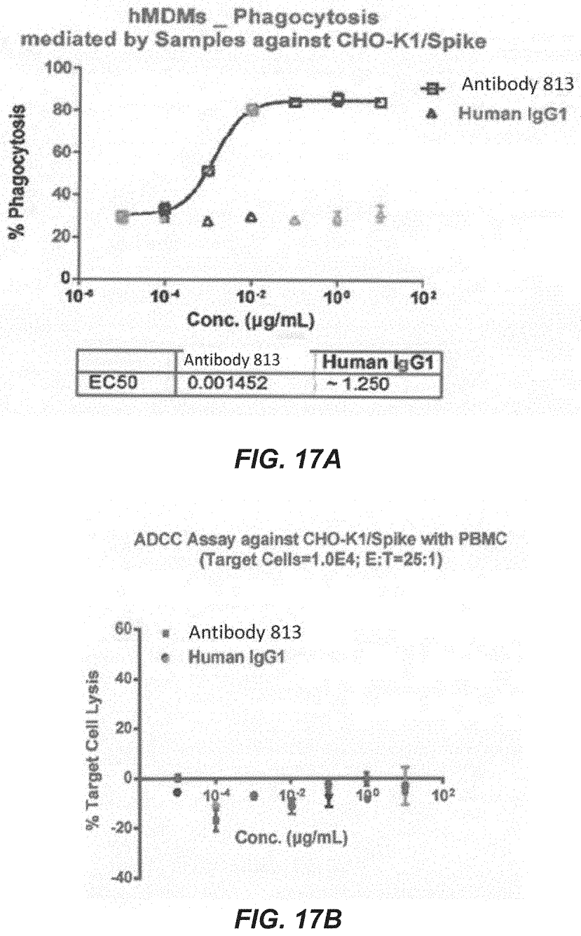

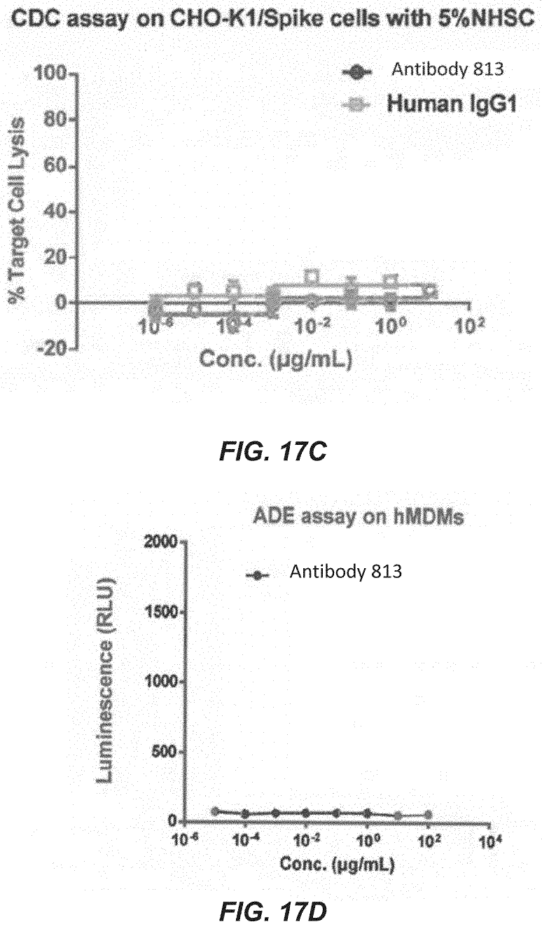

Several functionality-based assays were performed on Antibody 813. An assay for dose-dependent phagocytosis, which is dependent on engagement of the IgG1-Fc with CD32a (FCGRIIa)(FIG. 17A) showed that Antibody 813 was observed to induce ADCP effects against CHO-K1/Spike cells. The EC₅₀ value for Antibody 813 was determined to be 0.001452 μg/mL. An antibody-dependent cell-mediated cytotoxicity (ADCC) assay (FIG. 17B) showed no ADCC effects observed against CHO-K1/Spike cells. A complement-dependent cytotoxicity (CDC) assay (FIG. 17C) showed no CDC effects observed against CHO-K1/Spike cells. An antibody-dependent enhancement (ADE) assay (FIG. 17D) showed no ADE effects observed for Antibody 813. Overall, the functionality assays found that Antibody 813 shows ADCP as a secondary mechanism of action, but not ADCC, CDC, or ADE.

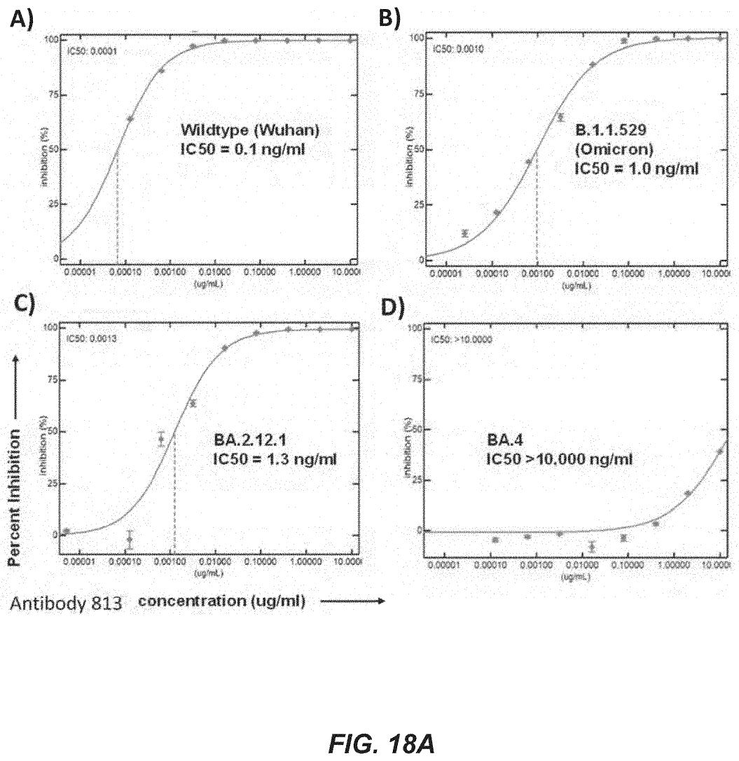

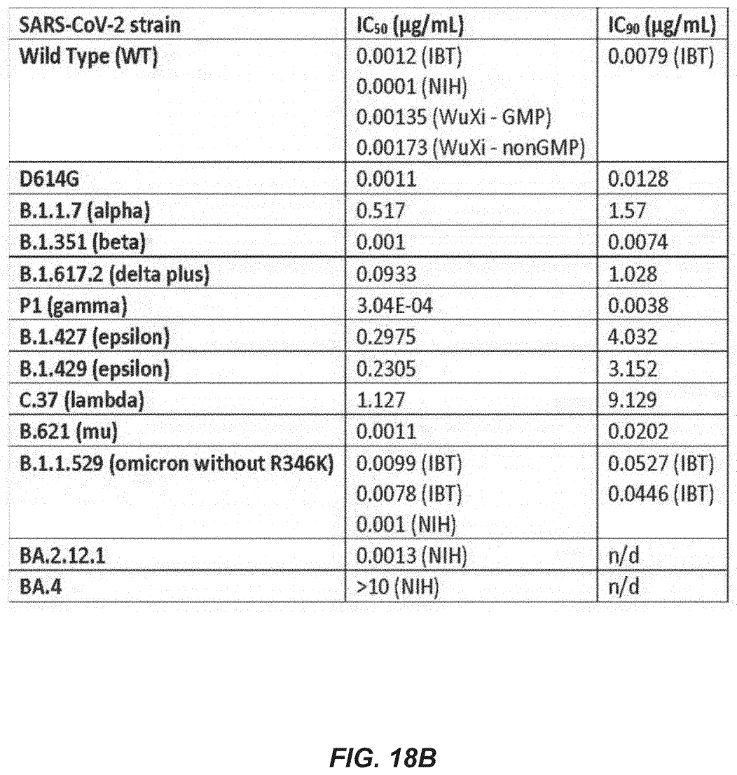

In vitro neutralization potency assays using pseudovirus were performed (FIGS. 18A-18B). Results show that Antibody 813 potently neutralizes the Ancestral SARS-CoV-2 variant with IC50 of <1 ng/ml and IC90 of 8 ng/ml by pseudovirus. Antibody 813 was also potent to prior variants of concern (e.g., beta, delta, gamma, mu, omicron; within 10× of Ancestral) including BA.2.12.1 but loses >1000× potency to BA.4.

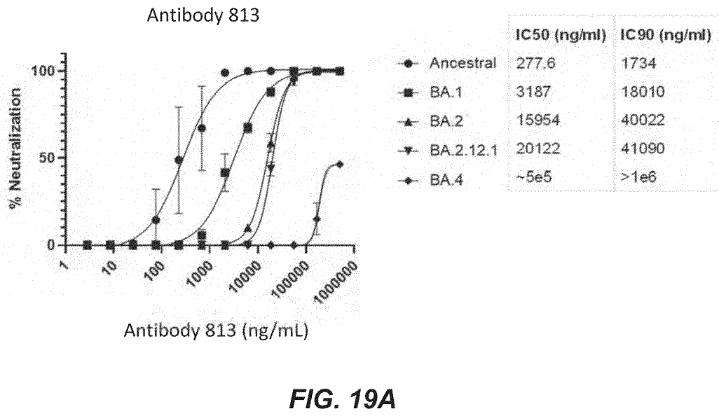

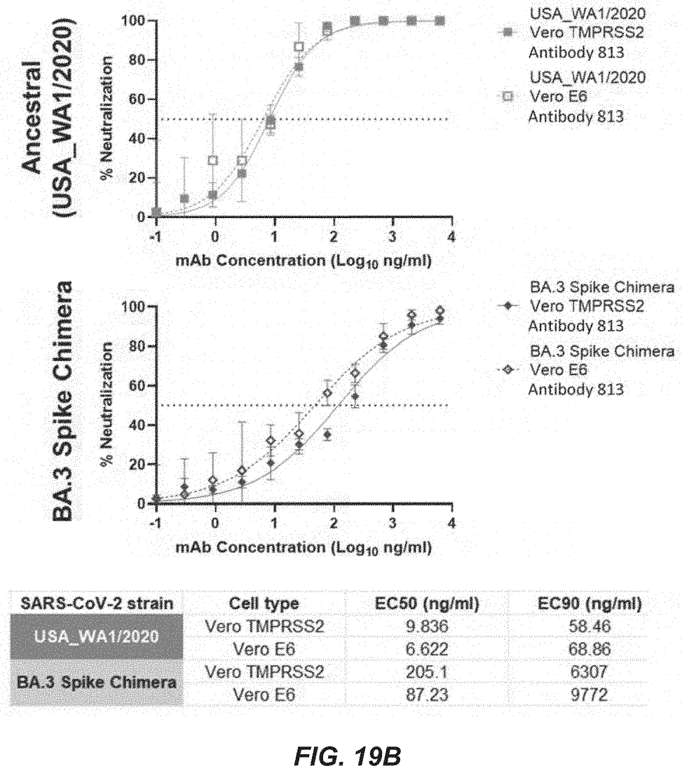

In vitro neutralization potency assays using authentic/live virus were also performed (FIGS. 19A-19B). Results show that Antibody 813 potently neutralizes live Ancestral SARS-CoV-2 with 1) IC50=6.6-9.8 ng/ml and IC90=69-58 ng/ml using Vero TMPRSS2 or Vero E6 cell types and 2) IC50=278 ng/ml and IC90=1734 ng/ml using Vero E6 TMPRSS2. Antibody 813 loses 13× potency to BA.3 vs. Ancestral. Antibody 813 also loses 11, 57, 72, and >1000 fold potency to BA.1, BA.2, BA.2.12.1, and BA.4 vs. Ancestral.

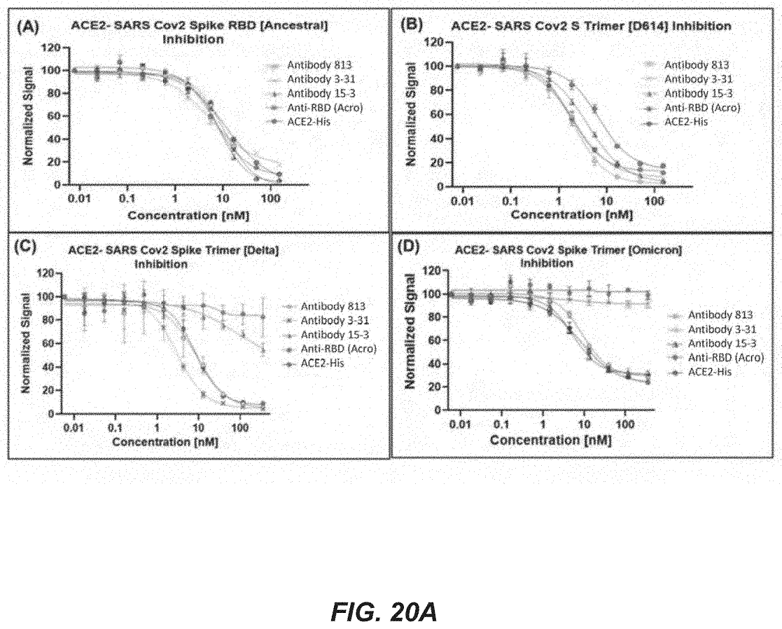

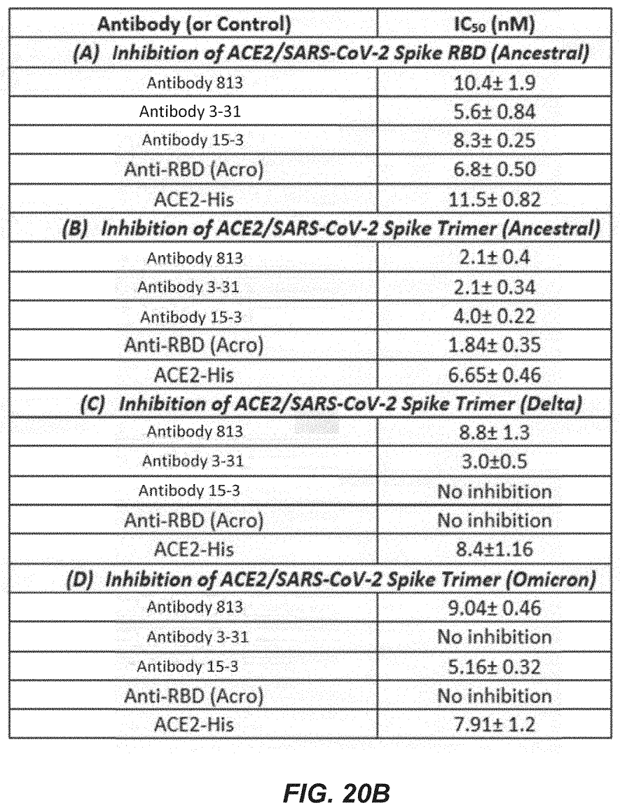

ACE2 Blockade experiments were performed using a biochemical inhibition assay (AlphaLISA) in order to determine IC50 values for the inhibition of the ACE2/SARS-CoV-2 Spike interactions by Antibody 813 (and Antibody 3-31 and Antibody 15-3, the bivalent monospecific parental VHH-Fc constructs from which the quadrivalent bispecific RBT-0813 is derived) (FIGS. 20A-20B). An anti-RBD neutralizing mAb from Acro Biosystems and ACE2-His were used as positive controls. Antibody 813 potently inhibited Ancestral spike RBD and SARS-CoV-2 spike trimers of Ancestral, Delta and Omicron variants.

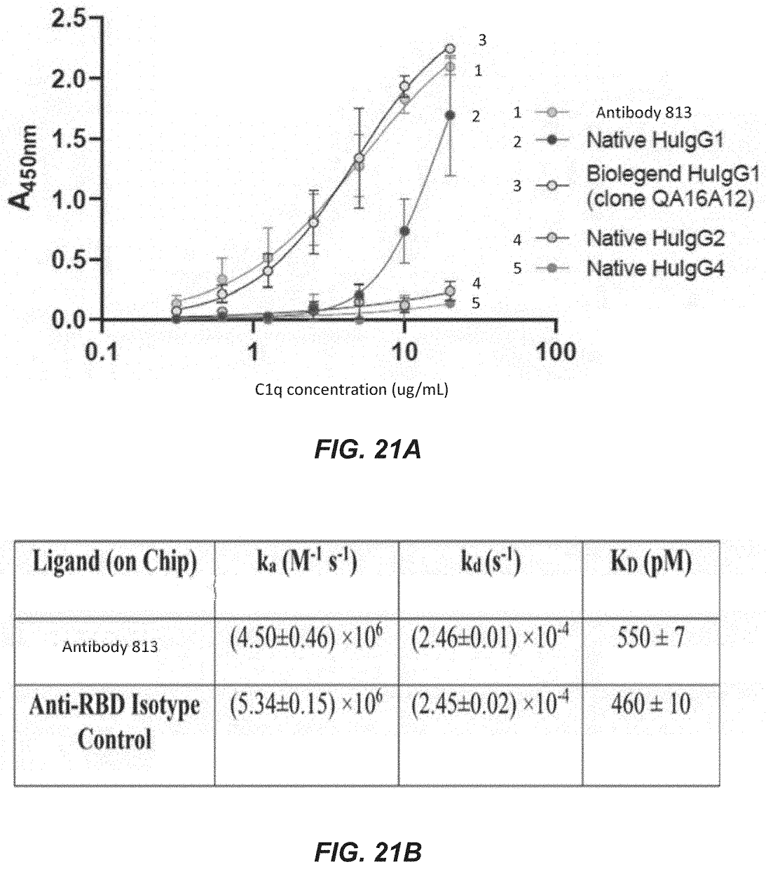

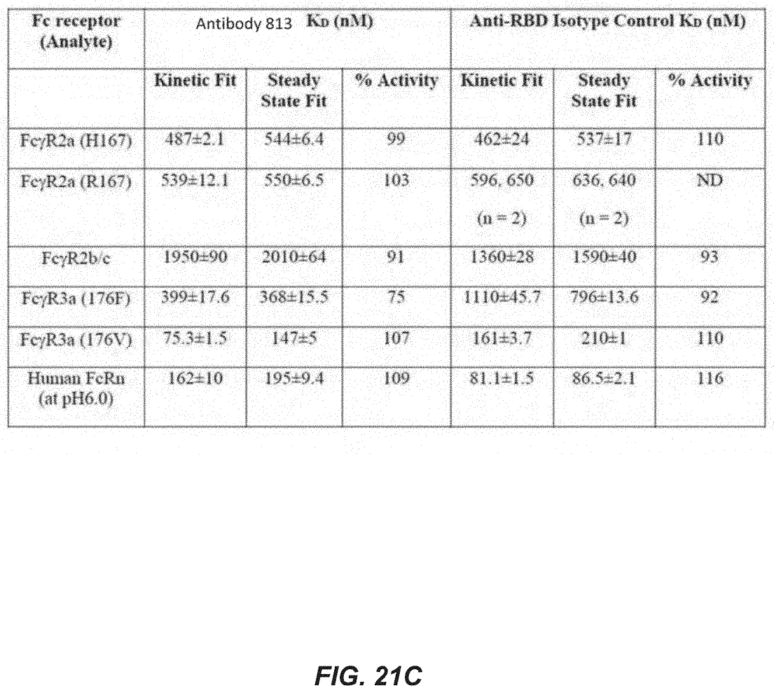

Fc functionality binding assays (FIGS. 21A-21C) were performed. Results showed that Antibody 813's interactions with Fc receptors (C1q, FCGR and FcRn) showed similar dose-dependent binding as a human IgG1 isotype control. These results suggest that Antibody 813 and the IgG1 control show 1) a similar ability to activate complement in-vivo (C1q); 2) a similar effector functions in vivo [FCGR interactions, including FCGR1, 2a (H/R 167), 2b/c, and 3a (F/V 176 isoforms]; and 3) a similar serum half life in vivo (FcRn).

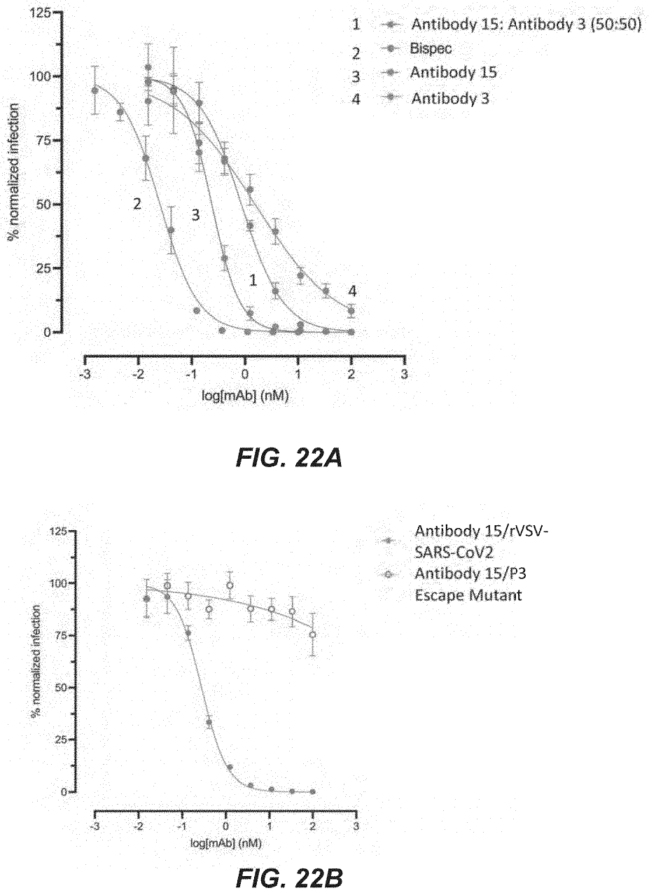

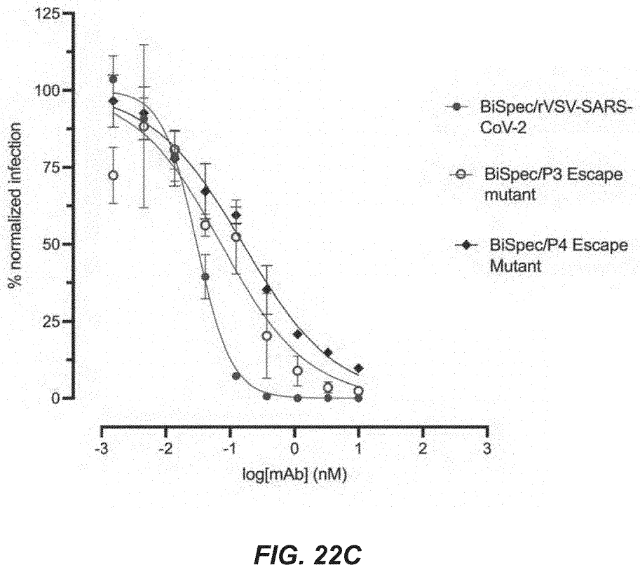

Antibody escape assay results tested plaque-purified escape mutants for Antibody 813 (bispecific) and the parental VHH-Fc constructs (FIGS. 22A-22C). The starling virus used was VSV-SARS-CoV-2 c10 containing mutations W64R, A372T (RBD) and H655Y. Results showed that antibody 813 (bispecific) was more potent than either the parental alone or as a cocktail.

### Example 8: Rat Studies of Antibody 813

These experiments test antibody 813 in rat an human tissue to determine clinical safety and preclinical PK/PD data in rats.

To obtain cross-tissue reactivity data, immunohistochemistry staining determined the binding activity of the biotinylated test article (Biotin-WBP2445) at 1 and 25 μg/mL with frozen normal human and Sprague Dawley rat tissues. Biotinylated control article (Biotin-Human IgG1, 1 and 25 μg/mL) and Phosphate Buffered Saline (PBS, 0.01 mol/L) were used as the isotype and reagent controls, respectively.

Results showed that in the human tissues, positive Biotin-Antibody 813 staining was observed in the cytoplasm of the histiocytes from 1/3 of the adrenal at 1 μg/mL, the staining intensity was weak, and the staining frequency was “rare”. At 25 μg/mL, positive Biotin-Antibody 813 staining was observed in the cytoplasm of the histiocytes from 1/3 of the fallopian tube, heart, esophagus, 2/3 of the bone marrows, 3/3 of the bladders, colons, livers (Kupffer cells), lymph nodes, small intestines, striated muscles. The staining intensity was weak to strong, and the staining frequency was “very rare” to “frequent”. There was no Biotin-WBP2495 staining in other human tissues.

In the Sprague Dawley rat tissues, no Biotin-Antibody 813 staining was observed at 1 μg/mL. Positive Biotin-Antibody 813 staining was observed at 25 μg/mL in the cytoplasm of the histiocytes from 3/3 of the lymph nodes. The staining intensity was weak, and the staining frequency was “rare” to “rare to occasional”. Positive Biotin-Antibody 813 staining was also observed in the cytoplasm of the histiocytes from 3/3 of the thymi. The staining intensity was weak, and the staining frequency was “rare”. There was no Biotin-Antibody 813 staining in other SD rat tissues. There was no Biotin-Human IgG1 staining observed in human or Sprague Dawley rat tissues.

The cross-tissue reactivity studies showed that positive cytoplasmic staining of Antibody 813 was observed in the histiocytes from human and SD rat tissues. The staining has little toxicological significance due to the limited accessibility of the test article to cytoplasmic structure in vivo.

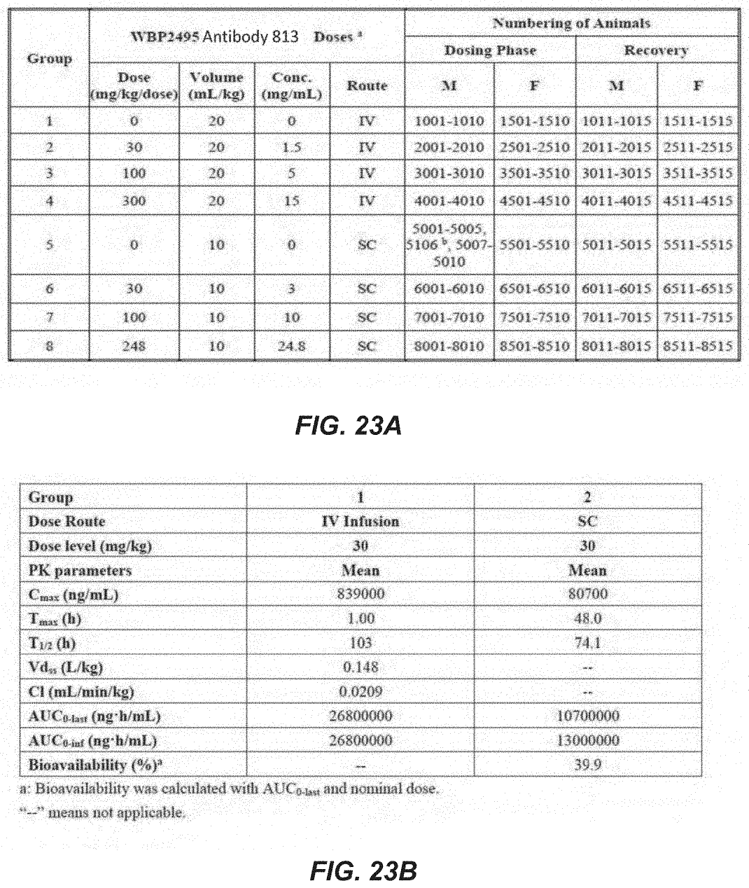

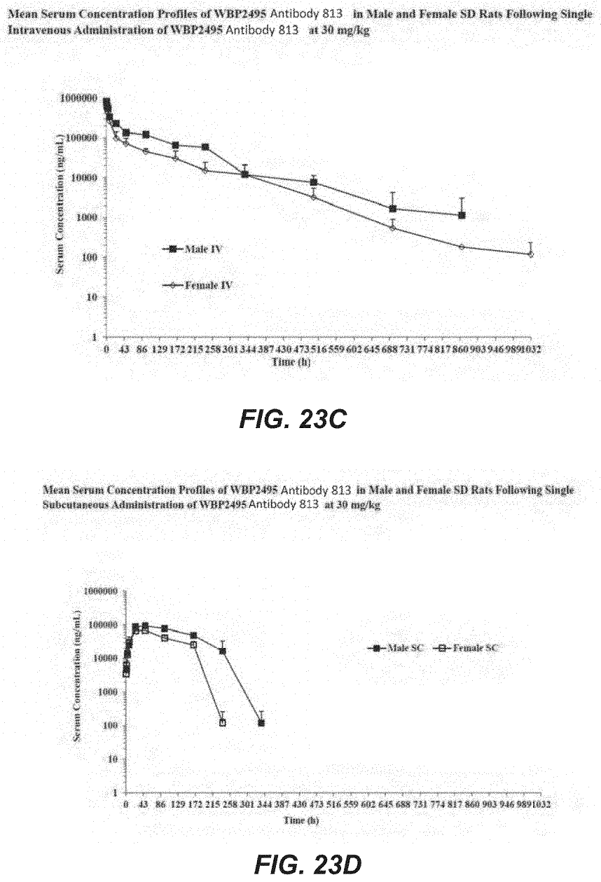

In vivo safety data was acquired through the administration of WBP2495 (Antibody 813 DS) to Sprague Dawley rats (FIG. 23A). Intravenous infusion at dosages of 0, 30, 100, or 300 mg/kg/dose and subcutaneous injection at dosages of 0, 30, 100, or 248 mg/kg/dose once weekly for 3 doses on Days 1, 8, and 15 followed by a 28-day recovery period was well tolerated. There were no test article related effects noted in this study. No observed adverse effect level (NOAEL) considered to be 300 mg/kg/dose via IV infusion or 248 mg/kg/dose via subcutaneous injection. The corresponding AUC0-168 h and Cmax of WBP2495 (Antibody 813 DS) following the last dose at 300 mg/kg/dose were 67500000 h*ng/mL and 4770000 ng/mL for males, and 46200000 h*ng/mL and 3950000 ng/mL for females, respectively. The corresponding AUC0-168 h and Cmax of WBP2495 (Antibody 813 DS) following the last dose at 248 mg/kg/dose were 1420000 h*ng/mL and 38700 ng/mL for males, and 3400000 h*ng/mL and 113000 ng/mL for females, respectively. Preclinical PK/PD data in rats was also obtained (FIGS. 23B-23D).

### Example 8: Clinical Studies of Antibody 813

Data from the rat PK study showed that the concentration of Antibody 813 remains above 3 μg/ml for approximately 21 days. The projected concentration-time profile for humans for 3 mg/kg Antibody 813 dose indicate that the concentrations of Antibody 813 remain above 3 μg/mL, at least for 10 days. Accordingly, the human efficacious dose is a human equivalent dose of 30 mg/kg in rats (i.e., 4.7 mg/kg).

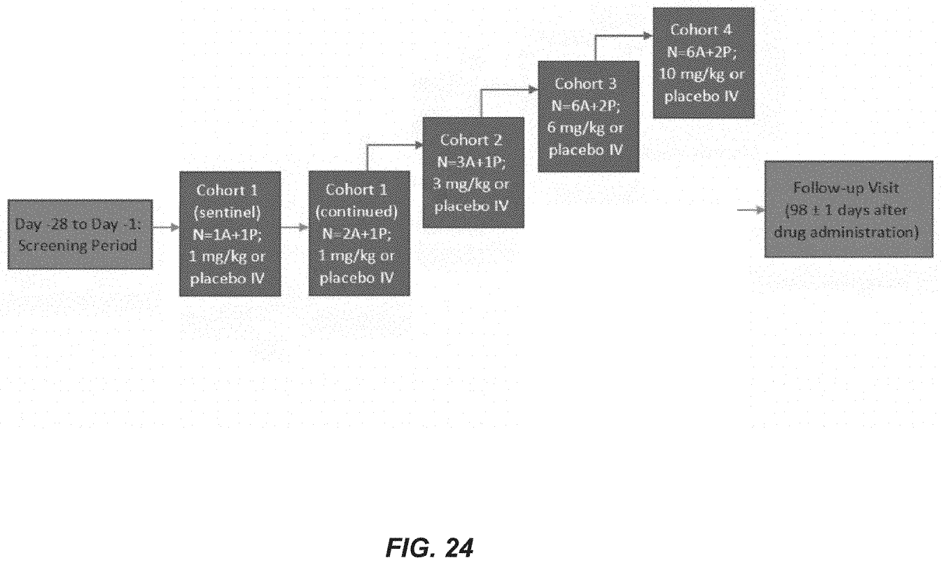

The phase 1 study (FIG. 24) is a randomized, double-blind, single-center, placebo-controlled, single ascending dose (SAD), first-in-human (FIH) study in healthy adult subjects. Subjects receive 1 of 4 SAD of Antibody 813 administered via approximately 1 hour IV infusion (I mg/kg, 3 mg/kg, 6 mg/kg, or 10 mg/kg) in one of a max of 4 cohorts.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An antibody or antibody fragment comprising a variable domain, heavy chain region (VH) and a variable domain, light chain region (VL), wherein the VH region comprises complementarity determining regions CDRH1, CDRH2, and CDRH3, and wherein (a) an amino acid sequence of CDRH1 is as set forth in any one of SEQ ID NOs: 1-89; (b) an amino acid sequence of CDRH2 is as set forth in any one of SEQ ID NOs: 90-178; (c) an amino acid sequence of CDRH3 is as set forth in any one of SEQ ID NOs: 179-267, and wherein the VL region comprise comprises complementarity determining regions CDRL1, CDRL2, and CDRL3, and wherein (a) an amino acid sequence of CDRL1 is as set forth in any one of SEQ ID NOs: 268-356; (b) an amino acid sequence of CDRL2 is as set forth in any one of SEQ ID NOs: 357-445; (c) an amino acid sequence of CDRL3 is as set forth in any one of SEQ ID NOs: 446-534.

2. The antibody or antibody fragment of claim 1, wherein the antibody or antibody fragment binds to a spike glycoprotein.

3. The antibody or antibody fragment of claim 1, wherein the antibody or antibody fragment binds to a receptor binding domain of the spike glycoprotein.

4. The antibody or antibody fragment of claim 1, wherein the antibody or antibody fragment comprises a K_{D} of less than 50 nM.

5-7. (canceled)

8. The antibody or antibody fragment of claim 1, wherein the antibody is a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, an isolated complementarity determining region (CDR), a diabody, a fragment comprised of only a single monomeric variable domain, disulfide-linked Fvs (sdFv), an intrabody, an anti-idiotypic (anti-Id) antibody, or ab antigen-binding fragments thereof.

9. The antibody or antibody fragment of claim 1, wherein the antibody is a single domain antibody.

10. An antibody or antibody fragment comprising a variable domain, heavy chain region (VH) comprising an amino acid sequence at least about 90% identical to a sequence as set forth in any one of SEQ ID NOs: 535-623 and a variable domain, light chain region (VL) comprising an amino acid sequence at least about 90% identical to a sequence as set forth in any one of SEQ ID NOs: 624-712.

11-18. (canceled)

19. A nucleic acid composition comprising: a) a first nucleic acid encoding a variable domain, heavy chain region (VH) comprising an amino acid sequence at least about 90% identical to a sequence as set forth in any one of SEQ ID NOs: 535-623; b) a second nucleic acid encoding a variable domain, light chain region (VL) comprising at least about 90% identical to a sequence as set forth in any one of SEQ ID NOs: 624-712; and an excipient.

20. A method of treating a SARS-CoV-2 infection, comprising administering the antibody or antibody fragment of claim 1.

21. The method of claim 20, wherein the antibody is administered prior to exposure to SARS-CoV-2.

22. The method of claim 21, wherein the antibody is administered at least about 1 week prior to exposure to SARS-CoV-2.

23-24. (canceled)

25. The method of claim 19, wherein the antibody is administered after exposure to SARS-CoV-2.

26-28. (canceled)

29. A method of treating an individual with a SARS-CoV-2 infection with the antibody or antibody fragment of claim 1 comprising:
a. obtaining or having obtained a sample from the individual;
b. performing or having performed an expression level assay on the sample to determine expression levels of SARS-CoV-2 antibodies; and
c. if the sample has an expression level of the SARS-CoV-2 antibodies then administering to the individual the antibody or antibody fragment of claim 1, thereby treating the SARS-CoV-2 infection.

30. A method for optimizing an antibody comprising:
a. providing a plurality of polynucleotide sequences encoding for an antibody or antibody fragment, wherein the antibody or antibody fragment is derived from a subject having SARS-CoV-2;
b. generating a nucleic acid library comprising the plurality of sequences that when translated encode for antibodies or antibody fragments that bind SARS-CoV-2 or ACE2 protein, wherein each of the sequences comprises a predetermined number of variants within a CDR relative to an input sequence that encodes an antibody; wherein the library comprises at least 50,000 variant sequences; and
c. synthesizing the at least 50,000 variant sequences.

31-34. (canceled)

35. The method of claim 30, wherein each sequence of the plurality of variant sequences comprises at least one variant in each CDR of a heavy chain or light chain, relative to the input sequence.

36. The method of claim 30, wherein each sequence of the plurality of variant sequences comprises at least two variants in each CDR of a heavy chain or light chain relative to the input sequence.

37. The method of claim 30, wherein at least one sequence when translated encodes for an antibody or antibody fragment having at least 5× higher binding affinity than a binding affinity of the input sequence.

38-39. (canceled)

40. The method of claim 30, wherein each sequence comprises at least one variant in each CDR of a heavy chain or light chain relative to a germline sequence of the input sequence.

41-42. (canceled)

43. An antibody or antibody fragment comprising an amino acid sequence at least about 90% identical to a sequence as set forth in SEQ ID NO: 713.

44-59. (canceled)

60. A method of treating an individual with a SARS-CoV-2 infection with the antibody or antibody fragment of claim 43 comprising:
a. obtaining or having obtained a sample from the individual;
b. performing or having performed an expression level assay on the sample to determine expression levels of SARS-CoV-2 antibodies; and
c. if the sample has an expression level of the SARS-CoV-2 antibodies then administering to the individual the antibody or antibody fragment of claim 43, thereby treating the SARS-CoV-2 infection.


SEQUENCE LISTING (753 sequences)

SEQ ID NO: 1 - protein, 9 aa, synthetic construct
FTFSSYAMH

SEQ ID NO: 2 - protein, 9 aa, synthetic construct
FSFNNYGMH

SEQ ID NO: 3 - protein, 9 aa, synthetic construct
FTFGTYAMH

SEQ ID NO: 4 - protein, 9 aa, synthetic construct
FDFSDYYMH

SEQ ID NO: 5 - protein, 9 aa, synthetic construct
FTFGTYAMH

SEQ ID NO: 6 - protein, 9 aa, synthetic construct
FTFSGYAMH

SEQ ID NO: 7 - protein, 9 aa, synthetic construct
FTFSSYAMH

SEQ ID NO: 8 - protein, 9 aa, synthetic construct
FTFSSYAMH

SEQ ID NO: 9 - protein, 9 aa, synthetic construct
FIFRSYAMH

SEQ ID NO: 10 - protein, 9 aa, synthetic construct
FTFSDYGMH

SEQ ID NO: 11 - protein, 9 aa, synthetic construct
FTFSNFPMH

SEQ ID NO: 12 - protein, 9 aa, synthetic construct
FTFSRFAMH

SEQ ID NO: 13 - protein, 9 aa, synthetic construct
FTFNNYAMH

SEQ ID NO: 14 - protein, 9 aa, synthetic construct
FTFSDYTMH

SEQ ID NO: 15 - skipped

SEQ ID NO: 16 - protein, 9 aa, synthetic construct
FTFSRYAMH

SEQ ID NO: 17 - protein, 9 aa, synthetic construct
FTFSRYAMH

SEQ ID NO: 18 - protein, 9 aa, synthetic construct
FTFSRYAMH

SEQ ID NO: 19 - protein, 9 aa, synthetic construct
FTFSRYAMH

SEQ ID NO: 20 - protein, 9 aa, synthetic construct
FTFSRYAMH

SEQ ID NO: 21 - protein, 9 aa, synthetic construct
FTFSRYAMH

SEQ ID NO: 22 - protein, 9 aa, synthetic construct
FTFGSYGMH

SEQ ID NO: 23 - protein, 9 aa, synthetic construct
FTFGTYAMH

SEQ ID NO: 24 - protein, 9 aa, synthetic construct
FTFSNFAMH

SEQ ID NO: 25 - protein, 9 aa, synthetic construct
FTFDNYAMH

SEQ ID NO: 26 - protein, 9 aa, synthetic construct
FTFSSFAMH

SEQ ID NO: 27 - protein, 9 aa, synthetic construct
FTFGTYAMH

SEQ ID NO: 28 - protein, 9 aa, synthetic construct
FTFSSYAMH

SEQ ID NO: 29 - protein, 9 aa, synthetic construct
FTFSSYAMH

SEQ ID NO: 30 - protein, 9 aa, synthetic construct
FTFGTYAMH

SEQ ID NO: 31 - protein, 9 aa, synthetic construct
FIFNNYGMH

SEQ ID NO: 32 - protein, 9 aa, synthetic construct
FTFDNYAMH

SEQ ID NO: 33 - protein, 9 aa, synthetic construct
FTFSSQAMH

SEQ ID NO: 34 - protein, 9 aa, synthetic construct
FSFSSYGMH

SEQ ID NO: 35 - protein, 9 aa, synthetic construct
FTFSSYSMH

SEQ ID NO: 36 - protein, 9 aa, synthetic construct
FIFSNYGMH

SEQ ID NO: 37 - protein, 9 aa, synthetic construct
FTFSDHYMH

SEQ ID NO: 38 - protein, 9 aa, synthetic construct
FIFSSYAMH

SEQ ID NO: 39 - protein, 9 aa, synthetic construct
FTFGTYAMH

SEQ ID NO: 40 - protein, 9 aa, synthetic construct
FTFSSYAMH

SEQ ID NO: 41 - protein, 9 aa, synthetic construct
FTFDDYAMH

SEQ ID NO: 42 - protein, 9 aa, synthetic construct
FTFSYYGMH

SEQ ID NO: 43 - protein, 9 aa, synthetic construct
FIFRSYAMH

SEQ ID NO: 44 - protein, 9 aa, synthetic construct
FTFSSYAMH

SEQ ID NO: 45 - protein, 9 aa, synthetic construct
FTFSYYGMH

SEQ ID NO: 46 - protein, 9 aa, synthetic construct
FTFSYYGMH

SEQ ID NO: 47 - protein, 9 aa, synthetic construct
FIFRNYAMH

SEQ ID NO: 48 - protein, 9 aa, synthetic construct
FTFSIYAMH

SEQ ID NO: 49 - protein, 9 aa, synthetic construct
FTFNNYVMH

SEQ ID NO: 50 - protein, 9 aa, synthetic construct
FTFSSYGMH

SEQ ID NO: 51 - protein, 9 aa, synthetic construct
FIFRSYAMH

SEQ ID NO: 52 - protein, 9 aa, synthetic construct
FSFGGYGMH

SEQ ID NO: 53 - protein, 9 aa, synthetic construct
FAFSSHAMH

SEQ ID NO: 54 - protein, 9 aa, synthetic construct
FSFSTYGMH

SEQ ID NO: 55 - protein, 9 aa, synthetic construct
FTFSSYSMH

SEQ ID NO: 56 - protein, 9 aa, synthetic construct
FSFGGYGMH

SEQ ID NO: 57 - protein, 9 aa, synthetic construct
FIFRSYAMH

SEQ ID NO: 58 - protein, 9 aa, synthetic construct
FTFGSYGMH

SEQ ID NO: 59 - protein, 9 aa, synthetic construct
FTFSSYAMH

SEQ ID NO: 60 - protein, 9 aa, synthetic construct
FAFSSYAMH

SEQ ID NO: 61 - protein, 9 aa, synthetic construct
FSFGGYGMH

SEQ ID NO: 62 - protein, 9 aa, synthetic construct
FSFGGYGMH

SEQ ID NO: 63 - protein, 9 aa, synthetic construct
FTFSSYGMH

SEQ ID NO: 64 - protein, 9 aa, synthetic construct
FIFSSYAMH

SEQ ID NO: 65 - protein, 9 aa, synthetic construct
FTFSNFPMH

SEQ ID NO: 66 - protein, 9 aa, synthetic construct
FTFSSYAMH

SEQ ID NO: 67 - protein, 9 aa, synthetic construct
FTFSNYAMH

SEQ ID NO: 68 - protein, 9 aa, synthetic construct
FSFGGYGMH

SEQ ID NO: 69 - protein, 9 aa, synthetic construct
FTFSSYAMH

SEQ ID NO: 70 - protein, 9 aa, synthetic construct
FSFSNYGMH

SEQ ID NO: 71 - protein, 9 aa, synthetic construct
FTFSGYAMH

SEQ ID NO: 72 - skipped

SEQ ID NO: 73 - protein, 9 aa, synthetic construct
FTFGTYAMH

SEQ ID NO: 74 - protein, 9 aa, synthetic construct
FTFSSYAMH

SEQ ID NO: 75 - protein, 9 aa, synthetic construct
FIFRSYAMH

SEQ ID NO: 76 - protein, 9 aa, synthetic construct
FTFSSSSMH

SEQ ID NO: 77 - protein, 9 aa, synthetic construct
FSFNNYGMH

SEQ ID NO: 78 - protein, 9 aa, synthetic construct
FTFSSYAMH

SEQ ID NO: 79 - protein, 9 aa, synthetic construct
FTFSSYAMH

SEQ ID NO: 80 - protein, 9 aa, synthetic construct
FTFSSYAMH

SEQ ID NO: 81 - protein, 9 aa, synthetic construct
FTFSSYAMH

SEQ ID NO: 82 - protein, 9 aa, synthetic construct
FIFSSYAMH

SEQ ID NO: 83 - protein, 9 aa, synthetic construct
FTFGSYGMH

SEQ ID NO: 84 - protein, 9 aa, synthetic construct
FIFSSYGMH

SEQ ID NO: 85 - protein, 9 aa, synthetic construct
FTFSSYSMH

SEQ ID NO: 86 - protein, 9 aa, synthetic construct
FTFSSYAMH

SEQ ID NO: 87 - protein, 9 aa, synthetic construct
FSFNNHAMH

SEQ ID NO: 88 - protein, 9 aa, synthetic construct
FAFDDYAMH

SEQ ID NO: 89 - protein, 9 aa, synthetic construct
FIFRSYAMH

SEQ ID NO: 90 - protein, 13 aa, synthetic construct
AVISYDGNHEYYA

SEQ ID NO: 91 - protein, 13 aa, synthetic construct
AVISFDGSNEYYA

SEQ ID NO: 92 - protein, 13 aa, synthetic construct
AVVSTEGGTTYYA

SEQ ID NO: 93 - protein, 13 aa, synthetic construct
AVISYDGSNKYYA

SEQ ID NO: 94 - protein, 13 aa, synthetic construct
AVVSTEGGTTYYA

SEQ ID NO: 95 - protein, 13 aa, synthetic construct
AVISYDGSNEYYA

SEQ ID NO: 96 - protein, 13 aa, synthetic construct
AVISYDGNHEYYA

SEQ ID NO: 97 - protein, 13 aa, synthetic construct
AVISYDGNHEYYA

SEQ ID NO: 98 - protein, 13 aa, synthetic construct
AVISYDGSSKYYA

SEQ ID NO: 99 - protein, 13 aa, synthetic construct
AVVSYDGTTKYYA

SEQ ID NO: 100 - protein, 13 aa, synthetic construct
AVISYDGSLKYYA

SEQ ID NO: 101 - protein, 13 aa, synthetic construct
AVISYDGSNKYYA

SEQ ID NO: 102 - protein, 13 aa, synthetic construct
AVISYDGNNKYYA

SEQ ID NO: 103 - protein, 13 aa, synthetic construct
AVISYEGSIKYYA

SEQ ID NO: 104 - skipped

SEQ ID NO: 105 - protein, 13 aa, synthetic construct
AVISYDGTNEYYA

SEQ ID NO: 106 - protein, 13 aa, synthetic construct
AVISYDGTNEYYA

SEQ ID NO: 107 - protein, 13 aa, synthetic construct
AVISYDGTNEYYA

SEQ ID NO: 108 - protein, 13 aa, synthetic construct
AVISYDGTNEYYA

SEQ ID NO: 109 - protein, 13 aa, synthetic construct
AVISYDGTNEYYA

SEQ ID NO: 110 - protein, 13 aa, synthetic construct
AVISYDGTNEYYA

SEQ ID NO: 111 - protein, 13 aa, synthetic construct
AVISYDGGDEYYA

SEQ ID NO: 112 - protein, 13 aa, synthetic construct
AVVSTEGGTTYYA

SEQ ID NO: 113 - protein, 13 aa, synthetic construct
AVISYDGNHEYYA

SEQ ID NO: 114 - protein, 13 aa, synthetic construct
AVISDDGRNKYYA

SEQ ID NO: 115 - protein, 13 aa, synthetic construct
AVISYDGSNKYYA

SEQ ID NO: 116 - protein, 13 aa, synthetic construct
AVVSTEGGTTYYA

SEQ ID NO: 117 - protein, 13 aa, synthetic construct
AVISYDGSNQYYA

SEQ ID NO: 118 - protein, 13 aa, synthetic construct
AVISYDGSNTYYA

SEQ ID NO: 119 - protein, 13 aa, synthetic construct
AVVSTEGGTTYYA

SEQ ID NO: 120 - protein, 13 aa, synthetic construct
AVISYDGSNIYYA

SEQ ID NO: 121 - protein, 13 aa, synthetic construct
AVISYDGSNKYYA

SEQ ID NO: 122 - protein, 13 aa, synthetic construct
AVISYDGSNKYYA

SEQ ID NO: 123 - protein, 13 aa, synthetic construct
AVISYDGSDKYYA

SEQ ID NO: 124 - protein, 13 aa, synthetic construct
AVISYDGSHKYYA

SEQ ID NO: 125 - protein, 13 aa, synthetic construct
AVISYDGSDKYYA

SEQ ID NO: 126 - protein, 13 aa, synthetic construct
AVISYDGSNEYYA

SEQ ID NO: 127 - protein, 13 aa, synthetic construct
AVISYDGSNKYYA

SEQ ID NO: 128 - protein, 13 aa, synthetic construct
AVISYDGNNKYYA

SEQ ID NO: 129 - protein, 13 aa, synthetic construct
AVISYDGTNDYYA

SEQ ID NO: 130 - protein, 13 aa, synthetic construct
AVISYDGSNKYYA

SEQ ID NO: 131 - protein, 13 aa, synthetic construct
AVTSYDGSNKYYA

SEQ ID NO: 132 - protein, 13 aa, synthetic construct
AVISYDGSSKYYA

SEQ ID NO: 133 - protein, 13 aa, synthetic construct
AVISYDGSNQYYA

SEQ ID NO: 134 - protein, 13 aa, synthetic construct
AVISYDGTNDYYA

SEQ ID NO: 135 - protein, 13 aa, synthetic construct
AVTSYDGSNKYYS

SEQ ID NO: 136 - protein, 13 aa, synthetic construct
AVISYDGSNKYYA

SEQ ID NO: 137 - protein, 13 aa, synthetic construct
AVISYDGTNEYYA

SEQ ID NO: 138 - protein, 13 aa, synthetic construct
AVISYDGTNDYYA

SEQ ID NO: 139 - protein, 13 aa, synthetic construct
AVISYDGTTEYYA

SEQ ID NO: 140 - protein, 13 aa, synthetic construct
AVISYDGSIKYYA

SEQ ID NO: 141 - protein, 13 aa, synthetic construct
AVISYDGSNEYYA

SEQ ID NO: 142 - protein, 13 aa, synthetic construct
AVISYDGSNKYYA

SEQ ID NO: 143 - protein, 13 aa, synthetic construct
AVISYDGSNKYYA

SEQ ID NO: 144 - protein, 13 aa, synthetic construct
AVISYDGSNKYYA

SEQ ID NO: 145 - protein, 13 aa, synthetic construct
AVISYDGSNKYYA

SEQ ID NO: 146 - protein, 13 aa, synthetic construct
AVISYDGSSKYYA

SEQ ID NO: 147 - protein, 13 aa, synthetic construct
AVISYDGNHEYYA

SEQ ID NO: 148 - protein, 13 aa, synthetic construct
AVISYDGSNQYYA

SEQ ID NO: 149 - protein, 13 aa, synthetic construct
AVISYDGTYEYYA

SEQ ID NO: 150 - protein, 13 aa, synthetic construct
AVISYDGSNKYYA

SEQ ID NO: 151 - protein, 13 aa, synthetic construct
AVISYDGSNKYYA

SEQ ID NO: 152 - protein, 13 aa, synthetic construct
AVISYDGNHEYYA

SEQ ID NO: 153 - protein, 13 aa, synthetic construct
AVISYDGSNKYYA

SEQ ID NO: 154 - protein, 13 aa, synthetic construct
AVISYDGNNKYYA

SEQ ID NO: 155 - protein, 13 aa, synthetic construct
AVISYDGSNQYYA

SEQ ID NO: 156 - protein, 13 aa, synthetic construct
AVISYDGSNEYYA

SEQ ID NO: 157 - protein, 13 aa, synthetic construct
AVISYDGSTKYYA

SEQ ID NO: 158 - protein, 13 aa, synthetic construct
AVISYDGTNEYYA

SEQ ID NO: 159 - protein, 13 aa, synthetic construct
AVISYDGNNKYYA

SEQ ID NO: 160 - protein, 13 aa, synthetic construct
AVISYDGSNKYYA

SEQ ID NO: 161 - skipped

SEQ ID NO: 162 - protein, 13 aa, synthetic construct
AVISYDGNNKYYA

SEQ ID NO: 163 - protein, 13 aa, synthetic construct
AVISYDGSNQYYA

SEQ ID NO: 164 - protein, 13 aa, synthetic construct
AVISYDGSSKYYA

SEQ ID NO: 165 - protein, 13 aa, synthetic construct
AVISYDGSNKYYA

SEQ ID NO: 166 - protein, 13 aa, synthetic construct
AVISYDGSNDYYA

SEQ ID NO: 167 - protein, 13 aa, synthetic construct
AVISYDGSNQYYA

SEQ ID NO: 168 - protein, 13 aa, synthetic construct
AVISYDGSNQYYA

SEQ ID NO: 169 - protein, 13 aa, synthetic construct
AVISYDGNHEYYA

SEQ ID NO: 170 - protein, 13 aa, synthetic construct
AVISYDGSNTYYA

SEQ ID NO: 171 - protein, 13 aa, synthetic construct
AVISYDGSNKYYA

SEQ ID NO: 172 - protein, 13 aa, synthetic construct
AVISYDGSHKYYA

SEQ ID NO: 173 - protein, 13 aa, synthetic construct
AVISYDGSNEYYA

SEQ ID NO: 174 - protein, 13 aa, synthetic construct
AVISYDGRNQYYA

SEQ ID NO: 175 - protein, 13 aa, synthetic construct
AVISYDGNNKYYA

SEQ ID NO: 176 - protein, 13 aa, synthetic construct
AVISYDGSDKYYA

SEQ ID NO: 177 - protein, 13 aa, synthetic construct
AVISYDGSNKYYA

SEQ ID NO: 178 - protein, 13 aa, synthetic construct
AVISYDGSSKYYA

SEQ ID NO: 179 - protein, 14 aa, synthetic construct
CARGYKGYYYMDVW

SEQ ID NO: 180 - protein, 13 aa, synthetic construct
CAKENWLGYFDPW

SEQ ID NO: 181 - protein, 12 aa, synthetic construct
CAGSYGAYFDYW

SEQ ID NO: 182 - protein, 13 aa, synthetic construct
CAREEPVYGMDVW

SEQ ID NO: 183 - protein, 12 aa, synthetic construct
CAGSYGAYFDYW

SEQ ID NO: 184 - protein, 16 aa, synthetic construct
CARTNSGSYYGPFDYW

SEQ ID NO: 185 - protein, 14 aa, synthetic construct
CARGYKGYYYMDVW

SEQ ID NO: 186 - protein, 14 aa, synthetic construct
CARGYKGYYYMDVW

SEQ ID NO: 187 - protein, 16 aa, synthetic construct
CARPSSGSYFPPFDYW

SEQ ID NO: 188 - protein, 13 aa, synthetic construct
CAKENWLGYFDPW

SEQ ID NO: 189 - protein, 12 aa, synthetic construct
CARYQGGYMDVW

SEQ ID NO: 190 - protein, 12 aa, synthetic construct
CARDTGLGFDPW

SEQ ID NO: 191 - protein, 16 aa, synthetic construct
CAKTMGGSYFDAFDIW

SEQ ID NO: 192 - protein, 16 aa, synthetic construct
CARSSSGSYPSLVDYW

SEQ ID NO: 193 - protein, 13 aa, synthetic construct
CARDYWVDYFKPG

SEQ ID NO: 194 - protein, 12 aa, synthetic construct
CARDTGLGFDPW

SEQ ID NO: 195 - protein, 12 aa, synthetic construct
CARDTGLGFDPW

SEQ ID NO: 196 - protein, 12 aa, synthetic construct
CARDTGLGFDPW

SEQ ID NO: 197 - protein, 12 aa, synthetic construct
CARDTGLGFDPW

SEQ ID NO: 198 - protein, 12 aa, synthetic construct
CARDTGLGFDPW

SEQ ID NO: 199 - protein, 12 aa, synthetic construct
CARDTGLGFDPW

SEQ ID NO: 200 - protein, 16 aa, synthetic construct
CARDISRYGYYGMDVW

SEQ ID NO: 201 - protein, 12 aa, synthetic construct
CAGSYGAYFDYW

SEQ ID NO: 202 - protein, 16 aa, synthetic construct
CAKTNSGSYGGMFDYW

SEQ ID NO: 203 - protein, 20 aa, synthetic construct
CAKDNYYDSSGYYGGGMDVW

SEQ ID NO: 204 - protein, 16 aa, synthetic construct
CARSRSGSYSSYFDYW

SEQ ID NO: 205 - protein, 16 aa, synthetic construct
CAGEYYDSSGSSIDYW

SEQ ID NO: 206 - protein, 16 aa, synthetic construct
CARAKGGGYRGAFDIW

SEQ ID NO: 207 - protein, 16 aa, synthetic construct
CARPRGGSYWTYFDYW

SEQ ID NO: 208 - protein, 12 aa, synthetic construct
CAGSYGAYFDYW

SEQ ID NO: 209 - protein, 19 aa, synthetic construct
CARDYNDGIGSYTGAFDSW

SEQ ID NO: 210 - protein, 11 aa, synthetic construct
CLREGILWDVW

SEQ ID NO: 211 - protein, 16 aa, synthetic construct
CAKTEGGTYGGAFDIW

SEQ ID NO: 212 - protein, 20 aa, synthetic construct
CAKDNYYDSSGYYGGGMDVW

SEQ ID NO: 213 - protein, 12 aa, synthetic construct
CARDGWGYFDYW

SEQ ID NO: 214 - protein, 13 aa, synthetic construct
CARDDYMYGFEHW

SEQ ID NO: 215 - protein, 13 aa, synthetic construct
CAKDLGPAGVDYW

SEQ ID NO: 216 - protein, 16 aa, synthetic construct
CARSRSGSYSSWFDYW

SEQ ID NO: 217 - protein, 16 aa, synthetic construct
CAKTGSGSYYSWFDYW

SEQ ID NO: 218 - protein, 16 aa, synthetic construct
CARTRGGSYFTPFDYW

SEQ ID NO: 219 - protein, 16 aa, synthetic construct
CASPHSGSYWAAFDIW

SEQ ID NO: 220 - protein, 16 aa, synthetic construct
CARPQGGSYFAAFDIW

SEQ ID NO: 221 - protein, 16 aa, synthetic construct
CARPSSGSYFPPFDYW

SEQ ID NO: 222 - protein, 16 aa, synthetic construct
CAKTRTGSYFSAFDIW

SEQ ID NO: 223 - protein, 16 aa, synthetic construct
CAKPHSGSYRGYFDYW

SEQ ID NO: 224 - protein, 16 aa, synthetic construct
CARPKSGSYATYFDYW

SEQ ID NO: 225 - protein, 16 aa, synthetic construct
CARPRGGSYHGAFDIW

SEQ ID NO: 226 - protein, 16 aa, synthetic construct
CAKSRGGSYYGAFDYW

SEQ ID NO: 227 - protein, 16 aa, synthetic construct
CARGESGSYWGAFDYW

SEQ ID NO: 228 - protein, 16 aa, synthetic construct
CARPSSGSYLGFFDYW

SEQ ID NO: 229 - protein, 16 aa, synthetic construct
CARTRGGSYYGAFDYW

SEQ ID NO: 230 - protein, 16 aa, synthetic construct
CAKSYSGSYSSYFDYW

SEQ ID NO: 231 - protein, 16 aa, synthetic construct
CAKAYSGSYMGYFDYW

SEQ ID NO: 232 - protein, 16 aa, synthetic construct
CARPLSGSYWSWFDPW

SEQ ID NO: 233 - protein, 16 aa, synthetic construct
CARGKGGGYYSSFDFW

SEQ ID NO: 234 - protein, 16 aa, synthetic construct
CARPYSGSYISWFDYW

SEQ ID NO: 235 - protein, 16 aa, synthetic construct
CARTLGGSYFAAFDIW

SEQ ID NO: 236 - protein, 16 aa, synthetic construct
CARPHSGSYTAYFDYW

SEQ ID NO: 237 - protein, 15 aa, synthetic construct
CARGYGGSYSYFDYW

SEQ ID NO: 238 - protein, 16 aa, synthetic construct
CARSLGGSYFSGMDVW

SEQ ID NO: 239 - protein, 16 aa, synthetic construct
CARSKGGSYYGPFDYW

SEQ ID NO: 240 - protein, 16 aa, synthetic construct
CARPKGGNYWNAFDIW

SEQ ID NO: 241 - protein, 16 aa, synthetic construct
CARPKSGSYVSYFDYW

SEQ ID NO: 242 - protein, 16 aa, synthetic construct
CARPRGGNYLNYFDYW

SEQ ID NO: 243 - protein, 13 aa, synthetic construct
CAKDHGDHYFDYW

SEQ ID NO: 244 - protein, 16 aa, synthetic construct
CARDKGGSYYGPFDYW

SEQ ID NO: 245 - protein, 16 aa, synthetic construct
CAKSGSGSYFSPFDYW

SEQ ID NO: 246 - protein, 16 aa, synthetic construct
CARPRGGSYKDAFDIW

SEQ ID NO: 247 - protein, 16 aa, synthetic construct
CARAHGGSYFSGMDVW

SEQ ID NO: 248 - protein, 16 aa, synthetic construct
CARSKGGSYYGPFDDW

SEQ ID NO: 249 - protein, 16 aa, synthetic construct
CARSRGGSYYAPFDYW

SEQ ID NO: 250 - protein, 16 aa, synthetic construct
CARPLGGSYFAAFDIW

SEQ ID NO: 251 - protein, 16 aa, synthetic construct
CAKTMSGSYFSAFDIW

SEQ ID NO: 252 - protein, 16 aa, synthetic construct
CARPHGGNYFDWFDPW

SEQ ID NO: 253 - protein, 16 aa, synthetic construct
CARPSGGSYFDPFDYW

SEQ ID NO: 254 - protein, 16 aa, synthetic construct
CAKVDSGSYVGYFDYW

SEQ ID NO: 255 - protein, 16 aa, synthetic construct
CARPNSGSYSNYFDYW

SEQ ID NO: 256 - protein, 16 aa, synthetic construct
CARSRSGSYLAYFDYW

SEQ ID NO: 257 - protein, 16 aa, synthetic construct
CARAAGGSYSSWFDPW

SEQ ID NO: 258 - protein, 16 aa, synthetic construct
CARAHSGSYFSHFDYW

SEQ ID NO: 259 - protein, 16 aa, synthetic construct
CARPTSGSYFSWFDPW

SEQ ID NO: 260 - protein, 16 aa, synthetic construct
CARPNSGSYWGPFDYW

SEQ ID NO: 261 - protein, 15 aa, synthetic construct
CARALGGNYYYFDYW

SEQ ID NO: 262 - protein, 16 aa, synthetic construct
CARPRSGSYLSAFDYW

SEQ ID NO: 263 - protein, 15 aa, synthetic construct
CAKGYGGNYYYMDGW

SEQ ID NO: 264 - protein, 16 aa, synthetic construct
CARTYGGSYYSAFDYW

SEQ ID NO: 265 - protein, 14 aa, synthetic construct
CARNLLRGYGMDVW

SEQ ID NO: 266 - protein, 13 aa, synthetic construct
CATLGYGDYPDYW

SEQ ID NO: 267 - protein, 16 aa, synthetic construct
CARPLGGGYQDAFDIW

SEQ ID NO: 268 - protein, 11 aa, synthetic construct
RASQGVSNYLA

SEQ ID NO: 269 - protein, 11 aa, synthetic construct
RASQSVSSSLA

SEQ ID NO: 270 - protein, 11 aa, synthetic construct
RASQSVNSYLA

SEQ ID NO: 271 - protein, 11 aa, synthetic construct
RASQSVGTSLA

SEQ ID NO: 272 - protein, 11 aa, synthetic construct
RATQYVNSYLA

SEQ ID NO: 273 - protein, 11 aa, synthetic construct
RASQSVGTSLA

SEQ ID NO: 274 - protein, 11 aa, synthetic construct
RASQGVSNYLA

SEQ ID NO: 275 - protein, 11 aa, synthetic construct
RASQGVSNYLA

SEQ ID NO: 276 - protein, 11 aa, synthetic construct
RASQSVDSRLA

SEQ ID NO: 277 - protein, 11 aa, synthetic construct
RASQSVRHHLA

SEQ ID NO: 278 - protein, 11 aa, synthetic construct
RASQSVGNFLA

SEQ ID NO: 279 - protein, 11 aa, synthetic construct
RASESISTYLA

SEQ ID NO: 280 - protein, 11 aa, synthetic construct
RASQSVGDFLA

SEQ ID NO: 281 - protein, 11 aa, synthetic construct
RASQTIRNSLN

SEQ ID NO: 282 - protein, 11 aa, synthetic construct
RASQSVSSSLA

SEQ ID NO: 283 - protein, 11 aa, synthetic construct
RASQDVSTYLA

SEQ ID NO: 284 - protein, 11 aa, synthetic construct
RASQDVSTYLA

SEQ ID NO: 285 - protein, 11 aa, synthetic construct
RASQDVSTYLA

SEQ ID NO: 286 - protein, 11 aa, synthetic construct
RASQDVSTYLA

SEQ ID NO: 287 - protein, 11 aa, synthetic construct
RASQDVSTYLA

SEQ ID NO: 288 - protein, 11 aa, synthetic construct
RASQDVSTYLA

SEQ ID NO: 289 - protein, 11 aa, synthetic construct
RASQSLGSFLA

SEQ ID NO: 290 - protein, 11 aa, synthetic construct
RASQSVNSYLA

SEQ ID NO: 291 - protein, 11 aa, synthetic construct
RASQNIGNHLA

SEQ ID NO: 292 - protein, 11 aa, synthetic construct
RASQSVGSYLA

SEQ ID NO: 293 - protein, 11 aa, synthetic construct
RASQSITDYLA

SEQ ID NO: 294 - protein, 11 aa, synthetic construct
RASQSVDSSLA

SEQ ID NO: 295 - protein, 11 aa, synthetic construct
RASQSIGSYLA

SEQ ID NO: 296 - protein, 11 aa, synthetic construct
RASQTVTNYLA

SEQ ID NO: 297 - protein, 11 aa, synthetic construct
RASQSVSYYLA

SEQ ID NO: 298 - protein, 11 aa, synthetic construct
RASQSVSTSLA

SEQ ID NO: 299 - protein, 11 aa, synthetic construct
RASHNINNFLA

SEQ ID NO: 300 - protein, 11 aa, synthetic construct
RASQSVGTSLA

SEQ ID NO: 301 - protein, 11 aa, synthetic construct
RASQSVSSQLA

SEQ ID NO: 302 - protein, 11 aa, synthetic construct
RASQSVDSRLA

SEQ ID NO: 303 - protein, 11 aa, synthetic construct
RASQSVGSYLA

SEQ ID NO: 304 - skipped

SEQ ID NO: 305 - protein, 11 aa, synthetic construct
RASQNIGSHLA

SEQ ID NO: 306 - protein, 11 aa, synthetic construct
RASQSLTSYLA

SEQ ID NO: 307 - protein, 11 aa, synthetic construct
RASQSIGSYLA

SEQ ID NO: 308 - protein, 11 aa, synthetic construct
RASQSVGSYLA

SEQ ID NO: 309 - protein, 11 aa, synthetic construct
RASQSVSSHLA

SEQ ID NO: 310 - protein, 11 aa, synthetic construct
RASQSVDSRLA

SEQ ID NO: 311 - protein, 11 aa, synthetic construct
RASQSVGDFLA

SEQ ID NO: 312 - protein, 11 aa, synthetic construct
RASQSVGSHLA

SEQ ID NO: 313 - protein, 11 aa, synthetic construct
RASQNVGQSLA

SEQ ID NO: 314 - protein, 11 aa, synthetic construct
RASQSLGNYLA

SEQ ID NO: 315 - protein, 11 aa, synthetic construct
RASQSLGNYLA

SEQ ID NO: 316 - protein, 11 aa, synthetic construct
RASQNIGNHLA

SEQ ID NO: 317 - protein, 11 aa, synthetic construct
RASQSVSTSLA

SEQ ID NO: 318 - protein, 11 aa, synthetic construct
RASQSVNSDLA

SEQ ID NO: 319 - protein, 11 aa, synthetic construct
RASQSVGSYLA

SEQ ID NO: 320 - protein, 11 aa, synthetic construct
RASQSVSSSLA

SEQ ID NO: 321 - protein, 11 aa, synthetic construct
RASQSVGSYLA

SEQ ID NO: 322 - protein, 11 aa, synthetic construct
RASQSVGSYLA

SEQ ID NO: 323 - protein, 11 aa, synthetic construct
RASQSVSSHLA

SEQ ID NO: 324 - protein, 11 aa, synthetic construct
RASQSLDSYLA

SEQ ID NO: 325 - protein, 11 aa, synthetic construct
RASQSVSKFLA

SEQ ID NO: 326 - protein, 11 aa, synthetic construct
RASQSIGGSLA

SEQ ID NO: 327 - protein, 11 aa, synthetic construct
RASQSISRYLA

SEQ ID NO: 328 - protein, 11 aa, synthetic construct
RASQSLGNYLA

SEQ ID NO: 329 - protein, 11 aa, synthetic construct
RASQSVNSDLA

SEQ ID NO: 330 - protein, 11 aa, synthetic construct
RASQSVRHHLA

SEQ ID NO: 331 - protein, 11 aa, synthetic construct
RASQSVDSRLA

SEQ ID NO: 332 - protein, 11 aa, synthetic construct
RASQSFGDSLA

SEQ ID NO: 333 - protein, 11 aa, synthetic construct
RASQSVNSYLA

SEQ ID NO: 334 - protein, 11 aa, synthetic construct
RASQSVSTSLA

SEQ ID NO: 335 - protein, 11 aa, synthetic construct
RASDTVSSYLA

SEQ ID NO: 336 - protein, 11 aa, synthetic construct
RASQSVRHHLA

SEQ ID NO: 337 - protein, 11 aa, synthetic construct
RASQIISSYLA

SEQ ID NO: 338 - protein, 11 aa, synthetic construct
RASHNIGTYLA

SEQ ID NO: 339 - skipped

SEQ ID NO: 340 - protein, 11 aa, synthetic construct
RASQSIGSYLA

SEQ ID NO: 341 - protein, 11 aa, synthetic construct
RASQDVSTYLA

SEQ ID NO: 342 - protein, 11 aa, synthetic construct
RASQSVGNFLA

SEQ ID NO: 343 - protein, 11 aa, synthetic construct
RASQRVSSYLA

SEQ ID NO: 344 - protein, 11 aa, synthetic construct
RASQGISTYLA

SEQ ID NO: 345 - protein, 11 aa, synthetic construct
RASESVSESLA

SEQ ID NO: 346 - protein, 11 aa, synthetic construct
RASQSVSTSLA

SEQ ID NO: 347 - protein, 11 aa, synthetic construct
RASESISTYLA

SEQ ID NO: 348 - protein, 11 aa, synthetic construct
RASQSVNSDLA

SEQ ID NO: 349 - protein, 11 aa, synthetic construct
RASQNVGQSLA

SEQ ID NO: 350 - protein, 11 aa, synthetic construct
RASQSVDSRLA

SEQ ID NO: 351 - protein, 11 aa, synthetic construct
RASQSVGKSLA

SEQ ID NO: 352 - protein, 11 aa, synthetic construct
RASQSVGDFLA

SEQ ID NO: 353 - protein, 11 aa, synthetic construct
RASDTVSSYLA

SEQ ID NO: 354 - protein, 11 aa, synthetic construct
RASQSIGDYLN

SEQ ID NO: 355 - protein, 11 aa, synthetic construct
RASQSITRYLN

SEQ ID NO: 356 - protein, 11 aa, synthetic construct
RASQSIGSYLA

SEQ ID NO: 357 - protein, 7 aa, synthetic construct
DASNRAT

SEQ ID NO: 358 - protein, 7 aa, synthetic construct
DASNRAT

SEQ ID NO: 359 - protein, 7 aa, synthetic construct
DVSNRAT

SEQ ID NO: 360 - protein, 7 aa, synthetic construct
GASNRAT

SEQ ID NO: 361 - protein, 7 aa, synthetic construct
DVSNRAT

SEQ ID NO: 362 - protein, 7 aa, synthetic construct
GASNRAT

SEQ ID NO: 363 - protein, 7 aa, synthetic construct
DASNRAT

SEQ ID NO: 364 - protein, 7 aa, synthetic construct
DASNRAT

SEQ ID NO: 365 - protein, 7 aa, synthetic construct
DTSNRAT

SEQ ID NO: 366 - protein, 7 aa, synthetic construct
DASNRAT

SEQ ID NO: 367 - protein, 7 aa, synthetic construct
DASNRAT

SEQ ID NO: 368 - protein, 7 aa, synthetic construct
DASNRAT

SEQ ID NO: 369 - protein, 7 aa, synthetic construct
DTSNRAT

SEQ ID NO: 370 - protein, 7 aa, synthetic construct
ASSSLQS

SEQ ID NO: 371 - protein, 7 aa, synthetic construct
DASNRAT

SEQ ID NO: 372 - protein, 7 aa, synthetic construct
DASNRAT

SEQ ID NO: 373 - protein, 7 aa, synthetic construct
DASNRAT

SEQ ID NO: 374 - protein, 7 aa, synthetic construct
DASNRAT

SEQ ID NO: 375 - protein, 7 aa, synthetic construct
DASNRAT

SEQ ID NO: 376 - protein, 7 aa, synthetic construct
DASNRAT

SEQ ID NO: 377 - protein, 7 aa, synthetic construct
DASNRAT

SEQ ID NO: 378 - protein, 7 aa, synthetic construct
DASNRAT

SEQ ID NO: 379 - protein, 7 aa, synthetic construct
DVSNRAT

SEQ ID NO: 380 - protein, 7 aa, synthetic construct
DASNRAT

SEQ ID NO: 381 - protein, 7 aa, synthetic construct
DAVNRAT

SEQ ID NO: 382 - protein, 7 aa, synthetic construct
DASNRAT

SEQ ID NO: 383 - protein, 7 aa, synthetic construct
DASNRAT

SEQ ID NO: 384 - protein, 7 aa, synthetic construct
DGSNRAT

SEQ ID NO: 385 - protein, 7 aa, synthetic construct
DTSNRAT

SEQ ID NO: 386 - protein, 7 aa, synthetic construct
DSSNRAT

SEQ ID NO: 387 - protein, 7 aa, synthetic construct
DATNRAT

SEQ ID NO: 388 - protein, 7 aa, synthetic construct
DTSNRAT

SEQ ID NO: 389 - protein, 7 aa, synthetic construct
GASNRAT

SEQ ID NO: 390 - protein, 7 aa, synthetic construct
DTSNRAT

SEQ ID NO: 391 - protein, 7 aa, synthetic construct
DASNRAT

SEQ ID NO: 392 - protein, 7 aa, synthetic construct
DAVNRAT

SEQ ID NO: 393 - skipped

SEQ ID NO: 394 - protein, 7 aa, synthetic construct
DVSNRAT

SEQ ID NO: 395 - protein, 7 aa, synthetic construct
DASNRAT

SEQ ID NO: 396 - protein, 7 aa, synthetic construct
DASNRAT

SEQ ID NO: 397 - protein, 7 aa, synthetic construct
DAVNRAT

SEQ ID NO: 398 - protein, 7 aa, synthetic construct
DVSNRAT

SEQ ID NO: 399 - protein, 7 aa, synthetic construct
DTSNRAT

SEQ ID NO: 400 - protein, 7 aa, synthetic construct
DTSNRAT

SEQ ID NO: 401 - protein, 7 aa, synthetic construct
DASNRAT

SEQ ID NO: 402 - protein, 7 aa, synthetic construct
DASNRAT

SEQ ID NO: 403 - protein, 7 aa, synthetic construct
DSSNRAT

SEQ ID NO: 404 - protein, 7 aa, synthetic construct
DSSNRAT

SEQ ID NO: 405 - protein, 7 aa, synthetic construct
DVSNRAT

SEQ ID NO: 406 - protein, 7 aa, synthetic construct
DATNRAT

SEQ ID NO: 407 - protein, 7 aa, synthetic construct
DASNRAT

SEQ ID NO: 408 - protein, 7 aa, synthetic construct
DAVNRAT

SEQ ID NO: 409 - protein, 7 aa, synthetic construct
DASNRAT

SEQ ID NO: 410 - protein, 7 aa, synthetic construct
DAVNRAT

SEQ ID NO: 411 - protein, 7 aa, synthetic construct
DAVNRAT

SEQ ID NO: 412 - protein, 7 aa, synthetic construct
DVSNRAT

SEQ ID NO: 413 - protein, 7 aa, synthetic construct
DVSNRAT

SEQ ID NO: 414 - protein, 7 aa, synthetic construct
DASNRAT

SEQ ID NO: 415 - protein, 7 aa, synthetic construct
DASNRAT

SEQ ID NO: 416 - protein, 7 aa, synthetic construct
DVSNRAT

SEQ ID NO: 417 - protein, 7 aa, synthetic construct
DSSNRAT

SEQ ID NO: 418 - protein, 7 aa, synthetic construct
DASNRAT

SEQ ID NO: 419 - protein, 7 aa, synthetic construct
DASNRAT

SEQ ID NO: 420 - protein, 7 aa, synthetic construct
DASNRAT

SEQ ID NO: 421 - protein, 7 aa, synthetic construct
DASNRAT

SEQ ID NO: 422 - protein, 7 aa, synthetic construct
DVSNRAT

SEQ ID NO: 423 - protein, 7 aa, synthetic construct
DISNRAT

SEQ ID NO: 424 - protein, 7 aa, synthetic construct
DTSNRAT

SEQ ID NO: 425 - protein, 7 aa, synthetic construct
DASNRAT

SEQ ID NO: 426 - protein, 7 aa, synthetic construct
DTSNRAT

SEQ ID NO: 427 - protein, 7 aa, synthetic construct
DVSNRAT

SEQ ID NO: 428 - skipped

SEQ ID NO: 429 - protein, 7 aa, synthetic construct
DVSNRAT

SEQ ID NO: 430 - protein, 7 aa, synthetic construct
DASNRAT

SEQ ID NO: 431 - protein, 7 aa, synthetic construct
DASNRAT

SEQ ID NO: 432 - protein, 7 aa, synthetic construct
DAFNRAT

SEQ ID NO: 433 - protein, 7 aa, synthetic construct
DASNRAT

SEQ ID NO: 434 - protein, 7 aa, synthetic construct
DASNRAT

SEQ ID NO: 435 - protein, 7 aa, synthetic construct
DATNRAT

SEQ ID NO: 436 - protein, 7 aa, synthetic construct
DASNRAT

SEQ ID NO: 437 - protein, 7 aa, synthetic construct
DASNRAT

SEQ ID NO: 438 - protein, 7 aa, synthetic construct
DASNRAT

SEQ ID NO: 439 - protein, 7 aa, synthetic construct
DTSNRAT

SEQ ID NO: 440 - protein, 7 aa, synthetic construct
DTSNRAT

SEQ ID NO: 441 - protein, 7 aa, synthetic construct
DTSNRAT

SEQ ID NO: 442 - protein, 7 aa, synthetic construct
DTSNRAT

SEQ ID NO: 443 - protein, 7 aa, synthetic construct
EASSLQS

SEQ ID NO: 444 - protein, 7 aa, synthetic construct
AASSLQS

SEQ ID NO: 445 - protein, 7 aa, synthetic construct
DVSNRAT

SEQ ID NO: 446 - protein, 10 aa, synthetic construct
CQQRYSWVTF

SEQ ID NO: 447 - protein, 11 aa, synthetic construct
CQQRINWPRSF

SEQ ID NO: 448 - protein, 10 aa, synthetic construct
CQQFSNWPTF

SEQ ID NO: 449 - protein, 10 aa, synthetic construct
CQQRSNWQPF

SEQ ID NO: 450 - protein, 10 aa, synthetic construct
CQQFSNWPTF

SEQ ID NO: 451 - protein, 10 aa, synthetic construct
CQLRSNWYTF

SEQ ID NO: 452 - protein, 10 aa, synthetic construct
CQQRYSWVTF

SEQ ID NO: 453 - protein, 10 aa, synthetic construct
CQQRYSWVTF

SEQ ID NO: 454 - protein, 11 aa, synthetic construct
CQQRSTWPPVF

SEQ ID NO: 455 - protein, 11 aa, synthetic construct
CQQRTDWPRAF

SEQ ID NO: 456 - protein, 11 aa, synthetic construct
CQQSSTWPLTF

SEQ ID NO: 457 - protein, 10 aa, synthetic construct
CQQRSGLITF

SEQ ID NO: 458 - protein, 9 aa, synthetic construct [sequence omitted]

SEQ ID NO: 459 - protein, 11 aa, synthetic construct
CQQTHSIPKTF

SEQ ID NO: 460 - protein, 11 aa, synthetic construct
CQQRINWPRSF

SEQ ID NO: 461 - protein, 11 aa, synthetic construct
CQQRRDWPQTF

SEQ ID NO: 462 - protein, 11 aa, synthetic construct
CQQRRDWPQTF

SEQ ID NO: 463 - protein, 11 aa, synthetic construct
CQQRRDWPQTF

SEQ ID NO: 464 - protein, 11 aa, synthetic construct
CQQRRDWPQTF

SEQ ID NO: 465 - protein, 11 aa, synthetic construct
CQQRRDWPQTF

SEQ ID NO: 466 - protein, 11 aa, synthetic construct
CQQRRDWPQTF

SEQ ID NO: 467 - protein, 12 aa, synthetic construct
CQQRALWPRLTF

SEQ ID NO: 468 - protein, 10 aa, synthetic construct
CQQFSNWPTF

SEQ ID NO: 469 - protein, 13 aa, synthetic construct
CQQRDNGPPEGTF

SEQ ID NO: 470 - protein, 11 aa, synthetic construct
CQQRFTWPTTF

SEQ ID NO: 471 - protein, 11 aa, synthetic construct
CHQRNNWPPTF

SEQ ID NO: 472 - protein, 11 aa, synthetic construct
CQQQSNWPGTF

SEQ ID NO: 473 - protein, 12 aa, synthetic construct
CQQRTNWPLFSF

SEQ ID NO: 474 - protein, 11 aa, synthetic construct
CQHRDDWPPTF

SEQ ID NO: 475 - protein, 11 aa, synthetic construct
CQQRSNWQGNF

SEQ ID NO: 476 - protein, 11 aa, synthetic construct
CQQHYSWPLTF

SEQ ID NO: 477 - protein, 12 aa, synthetic construct
CQQGRNWPPSSF

SEQ ID NO: 478 - protein, 11 aa, synthetic construct
CQERSNWPDTF

SEQ ID NO: 479 - protein, 11 aa, synthetic construct
CQQRYNWPSTF

SEQ ID NO: 480 - protein, 13 aa, synthetic construct
CQQRTNLPPSITF

SEQ ID NO: 481 - protein, 10 aa, synthetic construct
CQQRSDSITF

SEQ ID NO: 482 - skipped

SEQ ID NO: 483 - protein, 12 aa, synthetic construct
CQQRDYWPPYTF

SEQ ID NO: 484 - protein, 12 aa, synthetic construct
CQQRHYWPPITF

SEQ ID NO: 485 - protein, 11 aa, synthetic construct
CQQRDSWPHTF

SEQ ID NO: 486 - protein, 9 aa, synthetic construct
CQQRSLWPF

SEQ ID NO: 487 - protein, 9 aa, synthetic construct
CQQRDTFTF

SEQ ID NO: 488 - protein, 11 aa, synthetic construct
CQQRSTWPPVF

SEQ ID NO: 489 - protein, 9 aa, synthetic construct
CQYRSNFTF

SEQ ID NO: 490 - protein, 11 aa, synthetic construct
CQQISNWPLTF

SEQ ID NO: 491 - protein, 11 aa, synthetic construct
CQQRENWPPTF

SEQ ID NO: 492 - protein, 10 aa, synthetic construct
CQQRNWPYTF

SEQ ID NO: 493 - protein, 11 aa, synthetic construct
CQQRTDWPPSF

SEQ ID NO: 494 - protein, 12 aa, synthetic construct
CQQRKSWPPFTF

SEQ ID NO: 495 - protein, 11 aa, synthetic construct
CQRRTDWPPTF

SEQ ID NO: 496 - protein, 12 aa, synthetic construct
CQQRTDWPPATF

SEQ ID NO: 497 - protein, 11 aa, synthetic construct
CQQRFTWPTTF

SEQ ID NO: 498 - protein, 11 aa, synthetic construct
CQHRDDWPPTF

SEQ ID NO: 499 - protein, 12 aa, synthetic construct
CQQRNSWPPATF

SEQ ID NO: 500 - protein, 11 aa, synthetic construct
CQQVSNWPLTF

SEQ ID NO: 501 - protein, 12 aa, synthetic construct
CQVRSDWPPLTF

SEQ ID NO: 502 - protein, 12 aa, synthetic construct
CQQRRGWPPVTF

SEQ ID NO: 503 - protein, 11 aa, synthetic construct
CHQHSDWPLTF

SEQ ID NO: 504 - protein, 10 aa, synthetic construct
CQQRYSYFTF

SEQ ID NO: 505 - protein, 12 aa, synthetic construct
CQQSSNWPLFTF

SEQ ID NO: 506 - protein, 12 aa, synthetic construct
CQQRNTWPGVTF

SEQ ID NO: 507 - protein, 7 aa, synthetic construct
CQERSLF

SEQ ID NO: 508 - protein, 11 aa, synthetic construct
CQERSDWPITF

SEQ ID NO: 509 - protein, 11 aa, synthetic construct
CQQRSTWPPVF

SEQ ID NO: 510 - protein, 10 aa, synthetic construct
CQQRSIPITF

SEQ ID NO: 511 - protein, 12 aa, synthetic construct
CQERGNWPPFTF

SEQ ID NO: 512 - protein, 10 aa, synthetic construct
CQQRRSGLTF

SEQ ID NO: 513 - protein, 11 aa, synthetic construct
CQQRASWPLSF

SEQ ID NO: 514 - protein, 11 aa, synthetic construct
CQQSGSWPLTF

SEQ ID NO: 515 - protein, 12 aa, synthetic construct
CQVRSNWPPLTF

SEQ ID NO: 516 - protein, 11 aa, synthetic construct
CQQRADWPQTF

SEQ ID NO: 517 - skipped

SEQ ID NO: 518 - protein, 9 aa, synthetic construct
CQQRDSFTF

SEQ ID NO: 519 - protein, 11 aa, synthetic construct
CQQRAYWPGTF

SEQ ID NO: 520 - protein, 9 aa, synthetic construct
CQHRRLLTF

SEQ ID NO: 521 - protein, 11 aa, synthetic construct
CQQRQDWPLTF

SEQ ID NO: 522 - protein, 11 aa, synthetic construct
CQQRRRWPPTF

SEQ ID NO: 523 - protein, 10 aa, synthetic construct
CQQRTHGVTF

SEQ ID NO: 524 - protein, 11 aa, synthetic construct
CQQRQKWPLTF

SEQ ID NO: 525 - protein, 10 aa, synthetic construct
CQQRRNSLTF

SEQ ID NO: 526 - protein, 12 aa, synthetic construct
CQQRSTWSPLTF

SEQ ID NO: 527 - protein, 12 aa, synthetic construct
CQLRTNWPPVTF

SEQ ID NO: 528 - protein, 10 aa, synthetic construct
CQQRSSNWTF

SEQ ID NO: 529 - protein, 11 aa, synthetic construct
CQQRGSFPLTF

SEQ ID NO: 530 - protein, 10 aa, synthetic construct
CQQRSIRGTF

SEQ ID NO: 531 - protein, 11 aa, synthetic construct
CQQRGGWPPAF

SEQ ID NO: 532 - protein, 11 aa, synthetic construct
CLHTYLPPYSF

SEQ ID NO: 533 - protein, 11 aa, synthetic construct
CQQTYNFPHTF

SEQ ID NO: 534 - protein, 12 aa, synthetic construct
CQQRHHWPPVTF

SEQ ID NO: 535 - protein, 119 aa, synthetic construct
QVQLVESGGGVVQPGRSLRLSCAASGFTFSSYAMHWVRQAPGKGLEWVAVISYDGNHEYYADSVKGRFTISRDNSKNTLYLQMNSLRAEDTAVYYCARGYKGYYYMDVWGQGTLVTVSS

SEQ ID NO: 536 - protein, 118 aa, synthetic construct
QVQLVESGGGVVQPGRSLRLSCAASGFSFNNYGMHWVRQAPGKGLEWVAVISFDGSNEYYADSVKGRFTISRDNSKNTLYLQMNSLRAEDTAVYYCAKENWLGYFDPWGQGTLVTVSS

SEQ ID NO: 537 - protein, 117 aa, synthetic construct
QVQLVESGGGVVQPGRSLRLSCAASGFTFGTYAMHWVRQAPGKGLEWVAVVSTEGGTTYYADSVKGRFTISRDNSKNTLYLQMNSLRAEDTAVYYCAGSYGAYFDYWGQGTLVTVSS

SEQ ID NO: 538 - protein, 118 aa, synthetic construct
QVQLVESGGGVVQPGRSLRLSCAASGFDFSDYYMHWVRQAPGKGLEWVAVISYDGSNKYYADSVKGRFTISRDNSKNTLYLQMNSLRAEDTAVYYCAREEPVYGMDVWGQGTLVTVSS

SEQ ID NO: 539 - protein, 117 aa, synthetic construct; identical to SEQ ID NO: 537

SEQ ID NO: 540 - protein, 121 aa, synthetic construct
QVQLVESGGGVVQPGRSLRLSCAASGFTFSGYAMHWVRQAPGKGLEWVAVISYDGSNEYYADSVKGRFTISRDNSKNTLYLQMNSLRAEDTAVYYCARTNSGSYYGPFDYWGQGTLVTVSS

SEQ ID NO: 541 - protein, 119 aa, synthetic construct; identical to SEQ ID NO: 535

SEQ ID NO: 542 - protein, 119 aa, synthetic construct; identical to SEQ ID NO: 535

SEQ ID NO: 543 - protein, 121 aa, synthetic construct
QVQLVESGGGVVQPGRSLRLSCAASGFIFRSYAMHWVRQAPGKGLEWVAVISYDGSSKYYADSVKGRFTISRDNSKNTLYLQMNSLRAEDTAVYYCARPSSGSYFPPFDYWGQGTLVTVSS

SEQ ID NO: 544 - protein, 118 aa, synthetic construct
QVQLVESGGGVVQPGRSLRLSCAASGFTFSDYGMHWVRQAPGKGLEWVAVVSYDGTTKYYADSVKGRFTISRDNSKNTLYLQMNSLRAEDTAVYYCAKENWLGYFDPWGQGTLVTVSS

SEQ ID NO: 545 - protein, 117 aa, synthetic construct
QVQLVESGGGVVQPGRSLRLSCAASGFTFSNFPMHWVRQAPGKGLEWVAVISYDGSLKYYADSVKGRFTISRDNSKNTLYLQMNSLRAEDTAVYYCARYQGGYMDVWGQGTLVTVSS

SEQ ID NO: 546 - protein, 117 aa, synthetic construct
QVQLVESGGGVVQPGRSLRLSCAASGFTFSRFAMHWVRQAPGKGLEWVAVISYDGSNKYYADSVKGRFTISRDNSKNTLYLQMNSLRAEDTAVYYCARDTGLGFDPWGQGTLVTVSS

SEQ ID NO: 547 - protein, 121 aa, synthetic construct
QVQLVESGGGVVQPGRSLRLSCAASGFTFNNYAMHWVRQAPGKGLEWVAVISYDGNNKYYADSVKGRFTISRDNSKNTLYLQMNSLRAEDTAVYYCAKTMGGSYFDAFDIWGQGTLVTVSS

SEQ ID NO: 548 - protein, 121 aa, synthetic construct
QVQLVESGGGVVQPGRSLRLSCAASGFTFSDYTMHWVRQAPGKGLEWVAVISYEGSIKYYADSVKGRFTISRDNSKNTLYLQMNSLRAEDTAVYYCARSSSGSYPSLVDYWGQGTLVTVSS

SEQ ID NO: 549 - protein, 118 aa, synthetic construct
QVQLVESGGGVVQPGRSLRLSCAASGFSFSSYAMHWVRQAPGKGLEWVAVISFDGSNEYYADSVKGRFTISRDNSKNTLYLQMNSLRAEDTAVYYCARDYWVDYFKPGGRGALLTTSS

SEQ ID NO: 550 - protein, 117 aa, synthetic construct
QVQLVESGGGVVQPGRSLRLSCAASGFTFSRYAMHWVRQAPGKGLEWVAVISYDGTNEYYADSVKGRFTISRDNSKNTLYLQMNSLRAEDTAVYYCARDTGLGFDPWGQGTLVTVSS

SEQ ID NO: 551 - protein, 117 aa, synthetic construct; identical to SEQ ID NO: 550

SEQ ID NO: 552 - protein, 117 aa, synthetic construct; identical to SEQ ID NO: 550

SEQ ID NO: 553 - protein, 117 aa, synthetic construct; identical to SEQ ID NO: 550

SEQ ID NO: 554 - protein, 117 aa, synthetic construct; identical to SEQ ID NO: 550

SEQ ID NO: 555 - protein, 117 aa, synthetic construct; identical to SEQ ID NO: 550

SEQ ID NO: 556 - protein, 121 aa, synthetic construct
QVQLVESGGGVVQPGRSLRLSCAASGFTFGSYGMHWVRQAPGKGLEWVAVISYDGGDEYYADSVKGRFTISRDNSKNTLYLQMNSLRAEDTAVYYCARDISRYGYYGMDVWGQGTLVTVSS

SEQ ID NO: 557 - protein, 117 aa, synthetic construct; identical to SEQ ID NO: 537

SEQ ID NO: 558 - protein, 121 aa, synthetic construct
QVQLVESGGGVVQPGRSLRLSCAASGFTFSNFAMHWVRQAPGKGLEWVAVISYDGNHEYYADSVKGRFTISRDNSKNTLYLQMNSLRAEDTAVYYCAKTNSGSYGGMFDYWGQGTLVTVSS

SEQ ID NO: 559 - protein, 125 aa, synthetic construct
QVQLVESGGGVVQPGRSLRLSCAASGFTFDNYAMHWVRQAPGKGLEWVAVISDDGRNKYYADSVKGRFTISRDNSKNTLYLQMNSLRAEDTAVYYCAKDNYYDSSGYYGGGMDVWGQGTLVTVSS

SEQ ID NO: 560 - protein, 121 aa, synthetic construct
QVQLVESGGGVVQPGRSLRLSCAASGFTFSSFAMHWVRQAPGKGLEWVAVISYDGSNKYYADSVKGRFTISRDNSKNTLYLQMNSLRAEDTAVYYCARSRSGSYSSYFDYWGQGTLVTVSS

SEQ ID NO: 561 - protein, 121 aa, synthetic construct
QVQLVESGGGVVQPGRSLRLSCAASGFTFGTYAMHWVRQAPGKGLEWVAVVSTEGGTTYYADSVKGRFTISRDNSKNTLYLQMNSLRAEDTAVYYCAGEYYDSSGSSIDYWGQGTLVTVSS

SEQ ID NO: 562 - protein, 121 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCARAKGGGYRGAFDIWGQGTLVTVSS

SEQ ID NO: 563 - protein, 121 aa, synthetic construct
QVQLVESGGGVVQPGRSLRLSCAASGFTFSSYAMHWVRQAPGKGLEWVAVISYDGSNTYYADSVKGRFTISRDNSKNTLYLQMNSLRAEDTAVYYCARPRGGSYWTYFDYWGQGTLVTVSS

SEQ ID NO: 564 - protein, 117 aa, synthetic construct; identical to SEQ ID NO: 537

SEQ ID NO: 565 - protein, 124 aa, synthetic construct
QVQLVESGGGVVQPGRSLRLSCAASGFIFNNYGMHWVRQAPGKGLEWVAVISYDGSNIYYADSVKGRFTISRDNSKNTLYLQMNSLRAEDTAVYYCARDYNDGIGSYTGAFDSWGQGTLVTVSS

SEQ ID NO: 566 - protein, 116 aa, synthetic construct
QVQLVESGGGVVQPGRSLRLSCAASGFTFDNYAMHWVRQAPGKGLEWVAVISYDGSNKYYADSVKGRFTISRDNSKNTLYLQMNSLRAEDTAVYYCLREGILWDVWGQGTLVTVSS

SEQ ID NO: 567 - protein, 121 aa, synthetic construct
QVQLVESGGGVVQPGRSLRLSCAASGFTFSSQAMHWVRQAPGKGLEWVAVISYDGSNKYYADSVKGRFTISRDNSKNTLYLQMNSLRAEDTAVYYCAKTEGGTYGGAFDIWGQGTLVTVSS

SEQ ID NO: 568 - protein, 125 aa, synthetic construct
QVQLVESGGGVVQPGRSLRLSCAASGFSFSSYGMHWVRQAPGKGLEWVAVISYDGSDKYYADSVKGRFTISRDNSKNTLYLQMNSLRAEDTAVYYCAKDNYYDSSGYYGGGMDVWGQGTLVTVSS

SEQ ID NO: 569 - protein, 117 aa, synthetic construct
QVQLVESGGGVVQPGRSLRLSCAASGFTFSSYSMHWVRQAPGKGLEWVAVISYDGSHKYYADSVKGRFTISRDNSKNTLYLQMNSLRAEDTAVYYCARDGWGYFDYWGQGTLVTVSS

SEQ ID NO: 570 - protein, 118 aa, synthetic construct
QVQLVESGGGVVQPGRSLRLSCAASGFIFSNYGMHWVRQAPGKGLEWVAVISYDGSDKYYADSVKGRFTISRDNSKNTLYLQMNSLRAEDTAVYYCARDDYMYGFEHWGQGTLVTVSS

SEQ ID NO: 571 - protein, 118 aa, synthetic construct
QVQLVESGGGVVQPGRSLRLSCAASGFTFSDHYMHWVRQAPGKGLEWVAVISYDGSNEYYADSVKGRFTISRDNSKNTLYLQMNSLRAEDTAVYYCAKDLGPAGVDYWGQGTLVTVSS

SEQ ID NO: 572 - protein, 121 aa, synthetic construct
QVQLVESGGGVVQPGRSLRLSCAASGFIFSSYAMHWVRQAPGKGLEWVAVISYDGSNKYYADSVKGRFTISRDNSKNTLYLQMNSLRAEDTAVYYCARSRSGSYSSWFDYWGQGTLVTVSS

SEQ ID NO: 573 - protein, 121 aa, synthetic construct
QVQLVESGGGVVQPGRSLRLSCAASGFTFGTYAMHWVRQAPGKGLEWVAVISYDGNNKYYADSVKGRFTISRDNSKNTLYLQMNSLRAEDTAVYYCAKTGSGSYYSWFDYWGQGTLVTVSS

SEQ ID NO: 574 - protein, 121 aa, synthetic construct
QVQLVESGGGVVQPGRSLRLSCAASGFTFSSYAMHWVRQAPGKGLEWVAVISYDGTNDYYADSVKGRFTISRDNSKNTLYLQMNSLRAEDTAVYYCARTRGGSYFTPFDYWGQGTLVTVSS

SEQ ID NO: 575 - protein, 121 aa, synthetic construct
QVQLVESGGGVVQPGRSLRLSCAASGFTFDDYAMHWVRQAPGKGLEWVAVISYDGSNKYYADSVKGRFTISRDNSKNTLYLQMNSLRAEDTAVYYCASPHSGSYWAAFDIWGQGTLVTVSS

SEQ ID NO: 576 - protein, 121 aa, synthetic construct
QVQLVESGGGVVQPGRSLRLSCAASGFTFSYYGMHWVRQAPGKGLEWVAVTSYDGSNKYYADSVKGRFTISRDNSKNTLYLQMNSLRAEDTAVYYCARPQGGSYFAAFDIWGQGTLVTVSS

SEQ ID NO: 577 - protein, 121 aa, synthetic construct; identical to SEQ ID NO: 543

SEQ ID NO: 578 - protein, 121 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCAKTRTGSYFSAFDIWGQGTLVTVSS

SEQ ID NO: 579 - protein, 121 aa, synthetic construct
QVQLVESGGGVVQPGRSLRLSCAASGFTFSYYGMHWVRQAPGKGLEWVAVISYDGTNDYYADSVKGRFTISRDNSKNTLYLQMNSLRAEDTAVYYCAKPHSGSYRGYFDYWGQGTLVTVSS

SEQ ID NO: 580 - protein, 121 aa, synthetic construct
QVQLVESGGGVVQPGRSLRLSCAASGFTFSYYGMHWVRQAPGKGLEWVAVTSYDGSNKYYSDSVKGRFTISRDNSKNTLYLQMNSLRAEDTAVYYCARPKSGSYATYFDYWGQGTLVTVSS

SEQ ID NO: 581 - protein, 121 aa, synthetic construct
QVQLVESGGGVVQPGRSLRLSCAASGFIFRNYAMHWVRQAPGKGLEWVAVISYDGSNKYYADSVKGRFTISRDNSKNTLYLQMNSLRAEDTAVYYCARPRGGSYHGAFDIWGQGTLVTVSS

SEQ ID NO: 582 - protein, 121 aa, synthetic construct
QVQLVESGGGVVQPGRSLRLSCAASGFTFSIYAMHWVRQAPGKGLEWVAVISYDGTNEYYADSVKGRFTISRDNSKNTLYLQMNSLRAEDTAVYYCAKSRGGSYYGAFDYWGQGTLVTVSS

SEQ ID NO: 583 - protein, 121 aa, synthetic construct
QVQLVESGGGVVQPGRSLRLSCAASGFTFNNYVMHWVRQAPGKGLEWVAVISYDGTNDYYADSVKGRFTISRDNSKNTLYLQMNSLRAEDTAVYYCARGESGSYWGAFDYWGQGTLVTVSS

SEQ ID NO: 584 - protein, 121 aa, synthetic construct
QVQLVESGGGVVQPGRSLRLSCAASGFTFSSYGMHWVRQAPGKGLEWVAVISYDGTTEYYADSVKGRFTISRDNSKNTLYLQMNSLRAEDTAVYYCARPSSGSYLGFFDYWGQGTLVTVSS

SEQ ID NO: 585 - protein, 121 aa, synthetic construct
QVQLVESGGGVVQPGRSLRLSCAASGFIFRSYAMHWVRQAPGKGLEWVAVISYDGSIKYYADSVKGRFTISRDNSKNTLYLQMNSLRAEDTAVYYCARTRGGSYYGAFDYWGQGTLVTVSS

SEQ ID NO: 586 - protein, 121 aa, synthetic construct
QVQLVESGGGVVQPGRSLRLSCAASGFSFGGYGMHWVRQAPGKGLEWVAVISYDGSNEYYADSVKGRFTISRDNSKNTLYLQMNSLRAEDTAVYYCAKSYSGSYSSYFDYWGQGTLVTVSS

SEQ ID NO: 587 - protein, 121 aa, synthetic construct
QVQLVESGGGVVQPGRSLRLSCAASGFAFSSHAMHWVRQAPGKGLEWVAVISYDGSNKYYADSEKGRFTISRDNSKNTLYLQMNSLRAEDTAVYYCAKAYSGSYMGYFDYWGQGTLVTVSS

SEQ ID NO: 588 - protein, 121 aa, synthetic construct
QVQLVESGGGVVQPGRSLRLSCAASGFSFSTYGMHWVRQAPGKGLEWVAVISYDGSNKYYADSVKGRFTISRDNSKNTLYLQMNSLRAEDTAVYYCARPLSGSYWSWFDPWGQGTLVTVSS

SEQ ID NO: 589 - protein, 121 aa, synthetic construct
QVQLVESGGGVVQPGRSLRLSCAASGFTFSSYSMHWVRQAPGKGLEWVAVISYDGSNKYYADSVKGRFTISRDNSKNTLYLQMNSLRAEDTAVYYCARGKGGGYYSSFDFWGQGTLVTVSS

SEQ ID NO: 590 - protein, 121 aa, synthetic construct
QVQLVESGGGVVQPGRSLRLSCAASGFSFGGYGMHWVRQAPGKGLEWVAVISYDGSNKYYADSVKGRFTISRDNSKNTLYLQMNSLRAEDTAVYYCARPYSGSYISWFDYWGQGTLVTVSS

SEQ ID NO: 591 - protein, 121 aa, synthetic construct
QVQLVESGGGVVQPGRSLRLSCAASGFIFRSYAMHWVRQAPGKGLEWVAVISYDGSSKYYADSVKGRFTISRDNSKNTLYLQMNSLRAEDTAVYYCARTLGGSYFAAFDIWGQGTLVTVSS

SEQ ID NO: 592 - protein, 121 aa, synthetic construct
QVQLVESGGGVVQPGRSLRLSCAASGFTFGSYGMHWVRQAPGKGLEWVAVISYDGNHEYYADSVKGRFTISRDNSKNTLYLQMNSLRAEDTAVYYCARPHSGSYTAYFDYWGQGTLVTVSS

SEQ ID NO: 593 - protein, 120 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCARGYGGSYSYFDYWGQGTLVTVSS

SEQ ID NO: 594 - protein, 121 aa, synthetic construct
QVQLVESGGGVVQPGRSLRLSCAASGFAFSSYAMHWVRQAPGKGLEWVAVISYDGTYEYYADSVKGRFTISRDNSKNTLYLQMNSLRAEDTAVYYCARSLGGSYFSGMDVWGQGTLVTVSS

SEQ ID NO: 595 - protein, 121 aa, synthetic construct
QVQLVESGGGVVQPGRSLRLSCAASGFSFGGYGMHWVRQAPGKGLEWVAVISYDGSNKYYADSVKGRFTISRDNSKNTLYLQMNSLRAEDTAVYYCARSKGGSYYGPFDYWGQGTLVTVSS

SEQ ID NO: 596 - protein, 121 aa, synthetic construct
QVQLVESGGGVVQPGRSLRLSCAASGFSFGGYGMHWVRQAPGKGLEWVAVISYDGSNKYYADSVKGRFTISRDNSKNTLYLQMNSLRAEDTAVYYCARPKGGNYWNAFDIWGQGTLVTVSS

SEQ ID NO: 597 - protein, 121 aa, synthetic construct
QVQLVESGGGVVQPGRSLRLSCAASGFTFSSYGMHWVRQAPGKGLEWVAVISYDGNHEYYADSVKGRFTISRDNSKNTLYLQMNSLRAEDTAVYYCARPKSGSYVSYFDYWGQGTLVTVSS

SEQ ID NO: 598 - protein, 121 aa, synthetic construct
QVQLVESGGGVVQPGRSLRLSCAASGFIFSSYAMHWVRQAPGKGLEWVAVISYDGSNKYYADSVKGRFTISRDNSKNTLYLQMNSLRAEDTAVYYCARPRGGNYLNYFDYWGQGTLVTVSS

SEQ ID NO: 599 - protein, 118 aa, synthetic construct
QVQLVESGGGVVQPGRSLRLSCAASGFTFSNFPMHWVRQAPGKGLEWVAVISYDGNNKYYADSVKGRFTISRDNSKNTLYLQMNSLRAEDTAVYYCAKDHGDHYFDYWGQGTLVTVSS

SEQ ID NO: 600 - protein, 121 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCARDKGGSYYGPFDYWGQGTLVTVSS

SEQ ID NO: 601 - protein, 121 aa, synthetic construct
QVQLVESGGGVVQPGRSLRLSCAASGFTFSNYAMHWVRQAPGKGLEWVAVISYDGSNEYYADSEKGRFTISRDNSKNTLYLQMNSLRAEDTAVYYCAKSGSGSYFSPFDYWGQGTLVTVSS

SEQ ID NO: 602 - protein, 121 aa, synthetic construct
QVQLVESGGGVVQPGRSLRLSCAASGFSFGGYGMHWVRQAPGKGLEWVAVISYDGSTKYYADSVKGRFTISRDNSKNTLYLQMNSLRAEDTAVYYCARPRGGSYKDAFDIWGQGTLVTVSS

SEQ ID NO: 603 - protein, 121 aa, synthetic construct
QVQLVESGGGVVQPGRSLRLSCAASGFTFSSYAMHWVRQAPGKGLEWVAVISYDGTNEYYADSEKGRFTISRDNSKNTLYLQMNSLRAEDTAVYYCARAHGGSYFSGMDVWGQGTLVTVSS

SEQ ID NO: 604 - protein, 121 aa, synthetic construct
QVQLVESGGGVVQPGRSLRLSCAASGFSFSNYGMHWVRQAPGKGLEWVAVISYDGNNKYYADSVKGRFTISRDNSKNTLYLQMNSLRAEDTAVYYCARSKGGSYYGPFDDWGQGTLVTVSS

SEQ ID NO: 605 - protein, 121 aa, synthetic construct
QVQLVESGGGVVQPGRSLRLSCAASGFTFSGYAMHWVRQAPGKGLEWVAVISYDGSNKYYADSVKGRFTISRDNSKNTLYLQMNSLRAEDTAVYYCARSRGGSYYAPFDYWGQGTLVTVSS

SEQ ID NO: 606 - protein, 121 aa, synthetic construct
QVQLVESGGGVVQPGRSLRLSCAASGFTFSYYTMHWVRQAPGKGLEWVAVTSYDGSNKYYADSVKGRFTISRDNSKNTLYLQMNSLRAEDTAVYYCARPLGGSYFAAFDIWGQGTLVTVSS

SEQ ID NO: 607 - protein, 121 aa, synthetic construct
QVQLVESGGGVVQPGRSLRLSCAASGFTFGTYAMHWVRQAPGKGLEWVAVISYDGNNKYYADSVKGRFTISRDNSKNTLYLQMNSLRAEDTAVYYCAKTMSGSYFSAFDIWGQGTLVTVSS

SEQ ID NO: 608 - protein, 121 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCARPHGGNYFDWFDPWGQGTLVTVSS

SEQ ID NO: 609 - protein, 121 aa, synthetic construct
QVQLVESGGGVVQPGRSLRLSCAASGFIFRSYAMHWVRQAPGKGLEWVAVISYDGSSKYYADSVKGRFTISRDNSKNTLYLQMNSLRAEDTAVYYCARPSGGSYFDPFDYWGQGTLVTVSS

SEQ ID NO: 610 - protein, 121 aa, synthetic construct
QVQLVESGGGVVQPGRSLRLSCAASGFTFSSSSMHWVRQAPGKGLEWVAVISYDGSNKYYADSVKGRFTISRDNSKNTLYLQMNSLRAEDTAVYYCAKVDSGSYVGYFDYWGQGTLVTVSS

SEQ ID NO: 611 - protein, 121 aa, synthetic construct
QVQLVESGGGVVQPGRSLRLSCAASGFSFNNYGMHWVRQAPGKGLEWVAVISYDGSNDYYADSVKGRFTISRDNSKNTLYLQMNSLRAEDTAVYYCARPNSGSYSNYFDYWGQGTLVTVSS

SEQ ID NO: 612 - protein, 121 aa, synthetic construct
QVQLVESGGGVVQPGRSLRFSCAGTGFTFSSYAMHWVRQAPGKGLEWVAVISYDGSNQYYADSVKGRFTISRDNSKNTLYLQMNSLRAEDTAVYYCARSRSGSYLAYFDYWGQGTLVTVSS

SEQ ID NO: 613 - protein, 121 aa, synthetic construct
QVQLVESGGGVVQPGRSLRLSCAASGFTFSSYAMHWVRQAPGKGLEWVAVISYDGSNQYYADSVKGRFTISRDNSKNTLYLQMNSLRAEDTAVYYCARAAGGSYSSWFDPWGQGTLVTVSS

SEQ ID NO: 614 - protein, 121 aa, synthetic construct
QVQLVESGGGVVQPGRSLRLSCAASGFTFSSYAMHWVRQAPGKGLEWVAVISYDGNHEYYADSVKGRFTISRDNSKNTLYLQMNSLRAEDTAVYYCARAHSGSYFSHFDYWGQGTLVTVSS

SEQ ID NO: 615 - protein, 121 aa, synthetic construct
QVQLVESGGGVVQPGRSLRLSCAASGFTFSSYAMHWVRQAPGKGLEWVAVISYDGSNTYYADSVKGRFTISRDNSKNTLYLQMNSLRAEDTAVYYCARPTSGSYFSWFDPWGQGTLVTVSS

SEQ ID NO: 616 - protein, 121 aa, synthetic construct
QVQLVESGGGVVQPGRSLRLSCAASGFIFSSYAMHWVRQAPGKGLEWVAVISYDGSNKYYADSVKGRFTISRDNSKNTLYLQMNSLRAEDTAVYYCARPNSGSYWGPFDYWGQGTLVTVSS

SEQ ID NO: 617 - protein, 120 aa, synthetic construct
QVQLVESGGGVVQPGRSLRLSCAASGFTFGSYGMHWVRQAPGKGLEWVAVISYDGSHKYYADSVKGRFTISRDNSKNTLYLQMNSLRAEDTAVYYCARALGGNYYYFDYWGQGTLVTVSS

SEQ ID NO: 618 - protein, 121 aa, synthetic construct
QVQLVESGGGVVQPGRSLRLSCAASGFIFSSYGMHWVRQAPGKGLEWVAVISYDGSNEYYADSVKGRFTISRDNSKNTLYLQMNSLRAEDTAVYYCARPRSGSYLSAFDYWGQGTLVTVSS

SEQ ID NO: 619 - protein, 120 aa, synthetic construct
QVQLVESGGGVVQPGRSLRLSCAASGFTFSSYSMHWVRQAPGKGLEWVAVISYDGRNQYYADSVKGRFTISRDNSKNTLYLQMNSLRAEDTAVYYCAKGYGGNYYYMDGWGQGTLVTVSS

SEQ ID NO: 620 - protein, 121 aa, synthetic construct
QVQLVESGGGVVQPGRSLRLSCAASGFTFSSYAMHWVRQAPGKGLEWVAVISYDGNNKYYADSVKGRFTISRDNSKNTLYLQMNSLRAEDTAVYYCARTYGGSYYSAFDYWGQGTLVTVSS

SEQ ID NO: 621 - protein, 119 aa, synthetic construct
QVQLVESGGGVVQPGRSLRLSCAASGFSFNNHAMHWVRQAPGKGLEWVAVISYDGSDKYYADSVKGRFTISRDNSKNTLYLQMNSLRAEDTAVYYCARNLLRGYGMDVWGQGTLVTVSS

SEQ ID NO: 622 - protein, 118 aa, synthetic construct
QVQLVESGGGVVQPGRSLRLSCAASGFAFDDYAMHWVRQAPGKGLEWVAVISYDGSNKYYADSVKGRFTISRDNSKNTLYLQMNSLRAEDTAVYYCATLGYGDYPDYWGQGTLVTVSS

SEQ ID NO: 623 - protein, 121 aa, synthetic construct
QVQLVESGGGVVQPGRSLRLSCAASGFIFRSYAMHWVRQAPGKGLEWVAVISYDGSSKYYADSVKGRFTISRDNSKNTLYLQMNSLRAEDTAVYYCARPLGGGYQDAFDIWGQGTLVTVSS

SEQ ID NO: 624 - protein, 106 aa, synthetic construct
EIVLTQSPATLSLSPGERATLSCRASQGVSNYLAWYQQKPGQAPRLLIYDASNRATGIPARFSGSGSGTDFTLTISSLEPEDFAVYYCQQRYSWVTFGGGTKVEIK

SEQ ID NO: 625 - protein, 107 aa, synthetic construct
EIVLTQSPATLSLSPGERATLSCRASQSVSSSLAWYQQKPGQAPRLLIYDASNRATGIPARFSGSGSGTDFTLTISSLEPEDFAVYYCQQRINWPRSFGGGTKVEIK

SEQ ID NO: 626 - protein, 106 aa, synthetic construct
EIVLTQSPATLSLSPGERATLSCRASQSVNSYLAWYQQKPGQAPRLLIYDVSNRATGIPARFSGSGSGTDFTLTISSLEPEDFAVYYCQQFSNWPTFGGGTKVEIK

SEQ ID NO: 627 - protein, 106 aa, synthetic construct
EIVLTQSPATLSLSPGERATLSCRASQSVGTSLAWYQQKPGQAPRLLIYGASNRATGIPARFSGSGSGTDFTLTISSLEPEDFAVYYCQQRSNWQPFGGGTKVEIK

SEQ ID NO: 628 - protein, 106 aa, synthetic construct
EIVLTQSPATLSLSPGERATLSCRATQYVNSYLAWYQQKPRQAPRLIIYDVSNRATGIPARFSGSGSGTDFTLTISSLEPEDFAVYYCQQFSNWPTFGGGTKVEIK

SEQ ID NO: 629 - protein, 106 aa, synthetic construct
EIVLTQSPATLSLSPGERATLSCRASQSVGTSLAWYQQKPGQAPRLLIYGASNRATGIPARFSGSGSGTDFTLTISSLEPEDFAVYYCQLRSNWYTFGGGTKVEIK

SEQ ID NO: 630 - protein, 106 aa, synthetic construct
EIVLTQSPATLSLSPGERATLSCRASQGVSNYLAWYQQKPGQAPRLLIYDASNRATGIPARFSGSGSGTDFTLSISSLEPEDFAVYYCQQRYSWVTFGGGTKVEIK

SEQ ID NO: 631 - protein, 106 aa, synthetic construct; identical to SEQ ID NO: 624

SEQ ID NO: 632 - protein, 107 aa, synthetic construct
EIVLTQSPATLSLSPGERATLSCRASQSVDSRLAWYQQKPGQAPRLLIYDTSNRATGIPARFSGSGSGTDFTLTISSLEPEDFAVYYCQQRSTWPPVFGGGTKVEIK

SEQ ID NO: 633 - protein, 107 aa, synthetic construct
EIVLTQSPATLSLSPGERATLSCRASQSVRHHLAWYQQKPGQAPRLLIYDASNRATGIPARFSGSGSGTDFTLTISSLEPEDFAVYYCQQRTDWPRAFGGGTKVEIK

SEQ ID NO: 634 - protein, 107 aa, synthetic construct
EIVLTQSPATLSLSPGERATLSCRASQSVGNFLAWYQQKPGQAPRLLIYDASNRATGIPARFSGSGSGTDFTLTISSLEPEDFAVYYCQQSSTWPLTFGGGTKVEIK

SEQ ID NO: 635 - protein, 106 aa, synthetic construct
EIVLTQSPATLSLSPGERATLSCRASESISTYLAWYQQKPGQAPRLLIYDASNRATGIPARFSGSGSGTDFTLTISSLEPEDFAVYYCQQRSGLITFGGGTKVEIK

SEQ ID NO: 636 - protein, 105 aa, synthetic construct
EIVLTQSPATLSLSPGERATLSCRASQSVGDFLAWYQQKPGQAPRLLIYDTSNRATGIPARFSGSGSGTDFTLTISSLEPEDFAVYYCQQRSNLTFGGGTKVEIK

SEQ ID NO: 637 - protein, 107 aa, synthetic construct
DIQMTQSPSSLSASVGDRVTITCRASQTIRNSLNWYQQKPGKAPKLLIYASSSLQSGVPSRFSGSGSGTDFTLTISSLQPEDFATYYCQQTHSIPKTFGQGTKVEIK

SEQ ID NO: 638 - protein, 107 aa, synthetic construct; identical to SEQ ID NO: 625

SEQ ID NO: 639 - protein, 107 aa, synthetic construct
EIVLTQSPATLSLSPGERATLSCRASQDVSTYLAWYQQKPGQAPRLLIYDASNRATGIPARFSGSGSGTDFTLTISSLEPEDFAVYYCQQRRDWPQTFGGGTKVEIK

SEQ ID NO: 640 - protein, 107 aa, synthetic construct
EIVLTQSPATLSLSPGERATLSCRASQDVSTYLAWYQQKPGQAPRLLIYDASNRATGIPARFSGSGSGTDFTPFITNFEPEDFAVYYCQQRRDWPQTFGGGTKVEIK

SEQ ID NO: 641 - protein, 107 aa, synthetic construct; identical to SEQ ID NO: 640

SEQ ID NO: 642 - protein, 107 aa, synthetic construct
EIVLTQSPATLSLSPGERATLSCRASQDVSTYLAWYQQKPGQAPRLLIYDASNRATGIPARFSGSGSGTDFTLFITNFEPEDFAVYYCQQRRDWPQTFGGGTKVEIK

SEQ ID NO: 643 - protein, 107 aa, synthetic construct
EIVLTQSPATLSLSPGERATLSCRASQDVSTYLAWYQQKPGQAPRLLIYDASNRATGIPARFSGSGSGTDFTLTISSFEPEDFAVYYCQQRRDWPQTFGGGTKVEIK

SEQ ID NO: 644 - protein, 107 aa, synthetic construct
EIVLTQSPATLSLSPGERATLSCRASQDVSTYLAWYQQKPGQAPRLLIYDASNRATGIPARFSGSGSGTDFTPFITNFEHEDFAVYYCQQRRDWPQTFGGGTKVEIK

SEQ ID NO: 645 - protein, 108 aa, synthetic construct
EIVLTQSPATLSLSPGERATLSCRASQSLGSFLAWYQQKPGQAPRLLIYDASNRATGIPARFSGSGSGTDFTLTISSLEPEDFAVYYCQQRALWPRLTFGGGTKVEIK

SEQ ID NO: 646 - protein, 106 aa, synthetic construct; identical to SEQ ID NO: 626

SEQ ID NO: 647 - protein, 109 aa, synthetic construct
EIVLTQSPATLSLSPGERATLSCRASQNIGNHLAWYQQKPGQAPRLLIYDASNRATGIPARFSGSGSGTDFTLTISSLEPEDFAVYYCQQRDNGPPEGTFGGGTKVEIK

SEQ ID NO: 648 - protein, 107 aa, synthetic construct
EIVLTQSPATLSLSPGERATLSCRASQSVGSYLAWYQQKPGQAPRLLIYDAVNRATGIPARFSGSGSGTDFTLTISSLEPEDFAVYYCQQRFTWPTTFGGGTKVEIK

SEQ ID NO: 649 - protein, 107 aa, synthetic construct
EIVLTQSPATLSLSPGERATLSCRASQSITDYLAWYQQKPGQAPRLLIYDASNRATGIPARFSGSGSGTDFTLTISSLEPEDFAVYYCHQRNNWPPTFGGGTKVEIK

SEQ ID NO: 650 - protein, 107 aa, synthetic construct
EIVLTQSPATLSLSPGERATLSCRASQSVDSSLAWYQQKPGQAPRLLIYDASNRATGIPARFSGSGSGTDFTLTISSLEPEDFAVYYCQQQSNWPGTFGGGTKVEIK

SEQ ID NO: 651 - protein, 108 aa, synthetic construct
EIVLTQSPATLSLSPGERATLSCRASQSIGSYLAWYQQKPGQAPRLLIYDGSNRATGIPARFSGSGSGTDFTLTISSLEPEDFAVYYCQQRTNWPLFSFGGGTKVEIK

SEQ ID NO: 652 - protein, 107 aa, synthetic construct
EIVLTQSPATLSLSPGERATLSCRASQTVTNYLAWYQQKPGQAPRLLIYDTSNRATGIPARFSGSGSGTDFTLTISSLEPEDFAVYYCQHRDDWPPTFGGGTKVEIK

SEQ ID NO: 653 - protein, 107 aa, synthetic construct
EIVLTQSPATLSLSPGERATLSCRASQSVSYYLAWYQQKPGQAPRLLIYDSSNRATGIPARFSGSGSGTDFTLTISSLEPEDFAVYYCQQRSNWQGNFGGGTKVEIK

SEQ ID NO: 654 - protein, 107 aa, synthetic construct
EIVLTQSPATLSLSPGERATLSCRASQSVSTSLAWYQQKPGQAPRLLIYDATNRATGIPARFSGSGSGTDFTLTISSLEPEDFAVYYCQQHYSWPLTFGGGTKVEIK

SEQ ID NO: 655 - protein, 108 aa, synthetic construct
EIVLTQSPATLSLSPGERATLSCRASHNINNFLAWYQQKPGQAPRLLIYDTSNRATGIPARFSGSGSGTDFTLTISSLEPEDFAVYYCQQGRNWPPSSFGGGTKVEIK

SEQ ID NO: 656 - protein, 107 aa, synthetic construct
EIVLTQSPATLSLSPGERATLSCRASQSVGTSLAWYQQKPGQAPRLLIYGASNRATGIPARFSGSGSGTDFTLTISSLEPEDFAVYYCQERSNWPDTFGGGTKVEIK

SEQ ID NO: 657 - protein, 107 aa, synthetic construct
EIVLTQSPATLSLSPGERATLSCRASQSVSSQLAWYQQKPGQAPRLLMYDTSNRATGIPARFSGSGSGTDFTLTISSLEPEDFAVYYCQQRYNWPSTFGGGTKVEIK

SEQ ID NO: 658 - protein, 109 aa, synthetic construct
EIVLTQSPATLSLSPGERATLSCRASQSVDSRLAWYQQKPGQAPRLLIYDASNRATGIPARFSGSGSGTDFTLTISSLEPEDFAVYYCQQRTNLPPSITFGGGTKAKLK

SEQ ID NO: 659 - protein, 106 aa, synthetic construct
EMVVPQSPPTVSLSPGERATLSCRASQSVGSYLAWYQQKPGQAPRLLIYDAVNRATGIPARFSGSGSGTDFTLTISSLEPEDFAVYYCQQRSDSITFGGGTKVEIK

SEQ ID NO: 660 - protein, 107 aa, synthetic construct
EIVLTQSPATLSLSPGERATLSCRASQSLGRYLAWYQQKPGQAPRLLIYDSSNRATGIPARFSGSGSGTDFTLTISSLEPEDFAVYYCQQYGDWPETFGGGTKVEIK

SEQ ID NO: 661 - protein, 108 aa, synthetic construct
EIVLTQSPATLSLSPGERATLSCRASQNIGSHLAWYQQKPGQAPRLLIYDVSNRATGIPARFSGSGSGTDFTLTISSLEPEDFAVYYCQQRDYWPPYTFGGGTKVEIK

SEQ ID NO: 662 - protein, 108 aa, synthetic construct
EIVLTQSPATLSLSPGERATLSCRASQSLTSYLAWYQQKPGQAPRLLIYDASNRATGIPARFSGSGSGTDFTLTISSLEPEDFAVYYCQQRHYWPPITFGGGTKVEIK

SEQ ID NO: 663 - protein, 107 aa, synthetic construct
EIVLTQSPATLSLSPGERATLSCRASQSIGSYLAWYQQKPGQAPRLLIYDASNRATGIPARFSGSGSGTDFTLTISSLEPEDFAVYYCQQRDSWPHTFGGGTKVEIK

SEQ ID NO: 664 - protein, 105 aa, synthetic construct
EIVLTQSPATLSLSPGERATLSCRASQSVGSYLAWYQQKPGQAPRLLIYDAVNRATGIPARFSGSGSGTDFTLTISSLEPEDFAVYYCQQRSLWPFGGGTKVEIK

SEQ ID NO: 665 - protein, 105 aa, synthetic construct
EIVLTQSPATLSLSPGERATLSCRASQSVSSHLAWYQQKPGQAPRLLIYDVSNRATGIPARFSGSGSGTDFTLTISSLEPEDFAVYYCQQRDTFTFGGGTKVEIK

SEQ ID NO: 666 - protein, 107 aa, synthetic construct; identical to SEQ ID NO: 632

SEQ ID NO: 667 - protein, 105 aa, synthetic construct
EIVLTQSPATLSLSPGERATLSCRASQSVGDFLAWYQQKPGQAPRLLIYDTSNRATGIPARFSGSGSGTDFTLTISSLEPEDFAVYYCQYRSNFTFGGGTKVEIK

SEQ ID NO: 668 - protein, 107 aa, synthetic construct
EIVLTQSPATLSLSPGERATLSCRASQSVGSHLAWYQQKPGQAPRLLIYDASNRATGIPARFSGSGSGTDFTLTISSLEPEDFAVYYCQQISNWPLTFGGGTKVEIK

SEQ ID NO: 669 - protein, 107 aa, synthetic construct
EIVLTQSPATLSLSPGERATLSCRASQNVGQSLAWYQQKPGQAPRLLIYDASNRATGIPARFSGSGSGTDFTLTISSLEPEDFAVYYCQQRENWPPTFGGGTKVEIK

SEQ ID NO: 670 - protein, 106 aa, synthetic construct
EIVLTQSPATLSLSPGERATLSCRASQSLGNYLAWYQQKPGQAPRLLIYDSSNRATGIPARFSGSGSGTDFTLTISSLEPEDFAVYYCQQRNWPYTFGGGTKVEIK

SEQ ID NO: 671 - protein, 107 aa, synthetic construct
EIVLTQSPATLSLSPGERATLSCRASQSLGNYLAWYQQKPGQAPRLLIYDSSNRATGIPARFSGSGSGTDFTLTISSLEPEDFAVYYCQQRTDWPPSFGGGTKVEIK

SEQ ID NO: 672 - protein, 108 aa, synthetic construct
EIVLTQSPATLSLSPGERATLSCRASQNIGNHLAWYQQKPGQAPRLLIYDVSNRATGIPARFSGSGSGTDFTLTISSLEPEDFAVYYCQQRKSWPPFTFGGGTKVEIK

SEQ ID NO: 673 - protein, 107 aa, synthetic construct
EIVLTQSPATLSLSPGERATLSCRASQSVSTSLAWYQQKPGQAPRLLIYDATNRATGIPARFSGSGSGTDFTLTISSLEPEDFAVYYCQRRTDWPPTFGGGTKVEIK

SEQ ID NO: 674 - protein, 108 aa, synthetic construct
EIVLTQSPATLSLSPGERATLSCRASQSVNSDLAWYQQKPGQAPRLLIYDASNRATGIPARFSGSGSGTDFTLTISSLEPEDFAVYYCQQRTDWPPATFGGGTKVEIK

SEQ ID NO: 675 - protein, 107 aa, synthetic construct; identical to SEQ ID NO: 648

SEQ ID NO: 676 - protein, 107 aa, synthetic construct
EIVLTQSPATLSLSPGERATLSCRASQSVSSSLAWYQQKPGQAPRLLIYDASNRATGIPARFSGSGSGTDFTLTISSLEPEDFAVYYCQHRDDWPPTFGGGTKVEIK

SEQ ID NO: 677 - protein, 108 aa, synthetic construct
EIVLTQSPATLSLSPGERATLSCRASQSVGSYLAWYQQKPGQAPRLLIYDAVNRATGIPARFSGSGSGTDFTLTISSLEPEDFAVYYCQQRNSWPPATFGGGTKVEIK

SEQ ID NO: 678 - protein, 107 aa, synthetic construct
EIVLTQSPATLSLSPGERATLSCRASQSVGSYLAWYQQKPGQAPRLLIYDAVNRATGIPARFSGSGSGTDFTLTISSLEPEDFAVYYCQQVSNWPLTFGGGTKVEIK

SEQ ID NO: 679 - protein, 108 aa, synthetic construct
EIVLTQSPATLSLSPGERATLSCRASQSVSSHLAWYQQKPGQAPRLLIYDVSNRATGIPARFSGSGSGTDFTLTISSLEPEDFAVYYCQVRSDWPPLTFGGGTKVEIK

SEQ ID NO: 680 - protein, 108 aa, synthetic construct
EIVLTQSPATLSLSPGERATLSCRASQSLDSYLAWYQQKPGQAPRLLIYDVSNRATGIPARFSGSGSGTDFTLTISSLEPEDFAVYYCQQRRGWPPVTFGGGTKVEIK

SEQ ID NO: 681 - protein, 107 aa, synthetic construct
EIVLTQSPATLSLSPGERATLSCRASQSVSKFLAWYQQKPGQAPRLLIYDASNRATGIPARFSGSGSGTDFTLTISSLEPEDFAVYYCHQHSDWPLTFGGGTKVEIK

SEQ ID NO: 682 - protein, 106 aa, synthetic construct
EIVLTQSPATLSLSPGERATLSCRASQSIGGSLAWYQQKPGQAPRLLIYDASNRATGIPARFSGSGSGTDFTLTISSLEPEDFAVYYCQQRYSYFTFGGGTKVEIK

SEQ ID NO: 683 - protein, 108 aa, synthetic construct
EIVLTQSPATLSLSPGERATLSCRASQSISRYLAWYQQKPGQAPRLLIYDVSNRATGIPARFSGSGSGTDFTLTISSLEPEDFAVYYCQQSSNWPLFTFGGGTKVEIK

SEQ ID NO: 684 - protein, 108 aa, synthetic construct
EIVLTQSPATLSLSPGERATLSCRASQSLGNYLAWYQQKPGQAPRLLIYDSSNRATGIPARFSGSGSGTDFTLTISSLEPEDFAVYYCQQRNTWPGVTFGGGTKVEIK

SEQ ID NO: 685 - protein, 103 aa, synthetic construct
EIVLTQSPATLSLSPGERATLSCRASQSVNSDLAWYQQKPGQAPRLLIYDASNRATGIPARFSGSGSGTDFTLTISSLEPEDFAVYYCQERSLFGGGTKVEIK

SEQ ID NO: 686 - protein, 107 aa, synthetic construct
EIVLTQSPATLSLSPGERATLSCRASQSVRHHLAWYQQKPGQAPRLLIYDASNRATGIPARFSGSGSGTDFTLTISSLEPEDFAVYYCQERSDWPITFGGGTKVEIK

SEQ ID NO: 687 - protein, 107 aa, synthetic construct
EIVLTQSPATLSLSPGERATLSCRASQSVDSRLAWYQQKPGQAPRLLIYDASNRATGIPARFSGSGSGTDFTLTISSLEPEDFAVYYCQQRSTWPPVFGGGTKVEIK

SEQ ID NO: 688 - protein, 106 aa, synthetic construct
EIVLTQSPATLSLSPGERATLSCRASQSFGDSLAWYQQKPGQAPRLLIYDASNRATGIPARFSGSGSGTDFTLTISSLEPEDFAVYYCQQRSIPITFGGGTKVEIK

SEQ ID NO: 689 - protein, 108 aa, synthetic construct
EIVLTQSPATLSLSPGERATLSCRASQSVNSYLAWYQQKPGQAPRLLIYDVSNRATGIPARFSGSGSGTDFTLTISSLEPEDFAVYYCQERGNWPPFTFGGGTKVEIK

SEQ ID NO: 690 - protein, 106 aa, synthetic construct
EIVLTQSPATLSLSPGERATLSCRASQSVSTSLAWYQQKPGQAPRLLIYDISNRATGIPARFSGSGSGTDFTLTISSLEPEDFAVYYCQQRRSGLTFGGGTKVEIK

SEQ ID NO: 691 - protein, 107 aa, synthetic construct
EIVLTQSPATLSLSPGERATLSCRASDTVSSYLAWYQQKPGQAPRLLIYDTSNRATGIPARFSGSGSGTDFTLTISSLEPEDFAVYYCQQRASWPLSFGGGTKVEIK

SEQ ID NO: 692 - protein, 107 aa, synthetic construct
EIVLTQSPATLSLSPGERATLSCRASQSVRHHLAWYQQKPGQAPRLLIYDASNRATGIPARFSGSGSGTDFTLTISSLEPEDFAVYYCQQSGSWPLTFGGGTKVEIK

SEQ ID NO: 693 - protein, 108 aa, synthetic construct
EIVLTQSPATLSLSPGERATLSCRASQIISSYLAWYQQKPGQAPRLLIYDTSNRATGIPARFSGSGSGTDFTLTISSLEPEDFAVYYCQVRSNWPPLTFGGGTKVEIK

SEQ ID NO: 694 - protein, 107 aa, synthetic construct
EIVLTQSPATLSLSPGERATLSCRASHNIGTYLAWYQQKPGQAPRLLIYDVSNRATGIPARFSGSGSGTDFTLTISSLEPEDFAVYYCQQRADWPQTFGGGTKVEIK

SEQ ID NO: 695 - protein, 105 aa, synthetic construct
EIVLTQSPATLSLSPGERATLSCRASQSIGSYLAWYQQKPGQAPRLLIYDVSNRATGIPARFSGSGSGTDFTLTISSLEPEDFAVYYCQQRDSFTFGGGTKVEIK

SEQ ID NO: 696 - protein, 105 aa, synthetic construct; identical to SEQ ID NO: 695

SEQ ID NO: 697 - protein, 107 aa, synthetic construct
EIVLTQSPATLSLSPGERATLSCRASQDVSTYLAWYQQKPGQAPRLLIYDASNRATGIPARFSGSGSGTDFTLTISSLEPEDFAVYYCQQRAYWPGTFGGGTKVEIK

SEQ ID NO: 698 - protein, 105 aa, synthetic construct
EIVLTQSPATLSLSPGERATLSCRASQSVGNFLAWYQQKPGQAPRLLIYDASNRATGIPARFSGSGSGTDFTLTISSLEPEDFAVYYCQHRRLLTFGGGTKVEIK

SEQ ID NO: 699 - protein, 107 aa, synthetic construct
EIVLTQSPATLSLSPGERATLSCRASQRVSSYLAWYQQKPGQAPRLLIYDAFNRATGIPARFSGSGSGTDFTLTISSLEPEDFAVYYCQQRQDWPLTFGGGTKVEIK

SEQ ID NO: 700 - protein, 107 aa, synthetic construct
EIVLTQSPATLSLSPGERATLSCRASQGISTYLAWYQQKPGQAPRLLIYDASNRATGIPARFSGSGSGTDFTLTISSLEPEDFAVYYCQQRRRWPPTFGGGTKVEIK

SEQ ID NO: 701 - protein, 106 aa, synthetic construct
EIELTQSPATLSLSPGERATLSCRASESVSESLAWYQQKPGQAPRLLIYDASNRATGIPARFSGSGSGTDFTLTISSLEPEDFAVYYCQQRTHGVTFGGGTKVEIK

SEQ ID NO: 702 - protein, 107 aa, synthetic construct
EIVLTQSPATLSLSPGERATLSCRASQSVSTSLAWYQQKPGQAPRLLIYDATNRATGIPARFSGSGSGTDFTLTISSLEPEDFAVYYCQQRQKWPLTFGGGTKVEIK

SEQ ID NO: 703 - protein, 106 aa, synthetic construct
EIVLTQSPATLSLSPGERATLSCRASESISTYLAWYQQKPGQAPRLLIYDASNRATGIPARFSGSGSGTDFTLTISSLEPEDLAVYYCQQRRNSLTFGGGTKVEIK

SEQ ID NO: 704 - protein, 108 aa, synthetic construct
EIVLTQSPATLSLSPGERATLSCRASQSVNSDLAWYQQKPGQAPRLLIYDASNRATGIPARFSGSGSGTDFTLTISSLEPEDFAVYYCQQRSTWSPLTFGGGTKVEIK

SEQ ID NO: 705 - protein, 108 aa, synthetic construct
EIVLTQSPATLSLSPGERATLSCRASQNVGQSLAWYQQKPGQAPRLLIYDASNRATGIPARFSGSGSGTDFTLTISSLEPEDFAVYYCQLRTNWPPVTFGGGTKVEIK

SEQ ID NO: 706 - protein, 106 aa, synthetic construct
EIVLTQSPATLSLSPGERATLSCRASQSVDSRLAWYQQKPGQAPRLLIYDTSNRATGIPARFSGSGSGTDFTLTISSLEPEDFAVYYCQQRSSNWTFGGGTKVEIK

SEQ ID NO: 707 - protein, 107 aa, synthetic construct
EIVLTQSPATLSLSPGERATLSCRASQSVGKSLAWYQQKPGQAPRLLIYDTSNRATGIPARFSGSGSGTDFTLTISSLEPEDFAVYYCQQRGSFPLTFGGGTKVEIK

SEQ ID NO: 708 - protein, 106 aa, synthetic construct
EIVLTQSPATLSLSPGERATLSCRASQSVGDFLAWYQQKPGQAPRLLIYDTSNRATGIPARFSGSGSGTDFTLTISSLEPEDFAVYYCQQRSIRGTFGGGTKVEIK

SEQ ID NO: 709 - protein, 107 aa, synthetic construct
EIVLTQSPATLSLSPGERATLSCRASDTVSSYLAWYQQKPGQAPRLLIYDTSNRATGIPARFSGSGSGTDFTLTISSLEPEDFAVYYCQQRGGWPPAFGGGTKVEIK

SEQ ID NO: 710 - protein, 107 aa, synthetic construct
DIQMTQSPSSLSASVGDRVTITCRASQSIGDYLNWYQQKPGKAPKLLIYEASSLQSGVPSRFSGSGSGTDFTLTISSLQPEDFATYYCLHTYLPPYSFGQGTKVEIK

SEQ ID NO: 711 - protein, 107 aa, synthetic construct
DIQMTQSPSSLSASVGDRVTITCRASQSITRYLNWYQQKPGKAPKLLIYAASSLQSGVPSRFSGSGSGTDFTLTISSLQPEDFATYYCQQTYNFPHTFGQGTKVEIK

SEQ ID NO: 712 - protein, 108 aa, synthetic construct
EIVLTQSPATLSLSPGERATLSCRASQSIGSYLAWYQQKPGQAPRLLIYDVSNRATGIPARFSGSGSGTDFTLTISSFEPEDFAVYYCQQRHHWPPVTFGGGTKVEIK

SEQ ID NO: 713 - protein, 486 aa, synthetic construct
EVQLVESGGGLVQPGGSLRLSCAASGFTFSPSWMGWFRQAPGKEREFVATINEYGGRNYADSVKGRFTISADNSKNTAYLQMNSLKPEDTAVYYCARVDRDFDYWGQGTLVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSASEVQLVESGGGLVQPGGSLRLSCAASGSTFSINAMGWFRQAPGKEREFVAGITSSGGYTNYADSVKGRFTISADNSKNTAYLQMNSLKPEDTAVYYCAADGVPEYSDYASGPVWGQGTLVTVSS

SEQ ID NO: 714 - DNA, 62 bp, synthetic construct
  source 1..62: mol_type = other DNA
  modified_base 51: mod_base = OTHER; note = Thymidine-succinyl hexamide CED phosphoramidite
  modified_base 52: mod_base = OTHER; note = Thymidine-succinyl hexamide CED phosphoramidite
agacaatcaaccatttggggtggacagccttgacctctagacttcggcatnntttttttttt

SEQ ID NO: 715 - DNA, 112 bp, synthetic construct
  source 1..112: mol_type = other DNA
  modified_base 102: mod_base = OTHER; note = Thymidine-succinyl hexamide CED phosphoramidite
  modified_base 101: mod_base = OTHER; note = Thymidine-succinyl hexamide CED phosphoramidite
cgggatccttatcgtcatcgtcgtacagatcccgacccatttgctgtccaccagtcatgctagccataccatgatgatgatgatgatgagaaccccgcatnntttttttttt

SEQ ID NO: 716 - DNA, 19 bp, synthetic construct
  source 1..19: mol_type = other DNA
atgcggggttctcatcatc

SEQ ID NO: 717 - DNA, 20 bp, synthetic construct
  source 1..20: mol_type = other DNA
cgggatccttatcgtcatcg

SEQ ID NO: 718 - protein, 18 aa, synthetic construct
CAADGVPEYSDYASGPVW

SEQ ID NO: 719 - protein, 21 aa, synthetic construct
  source 1..21: note = VHHh Shuffle
CAAALSEVWRGSENLREGYDW

SEQ ID NO: 720 - protein, 18 aa, synthetic construct
  source 1..18: note = VHHh Shuffle
CAADRAADFFAQRDEYDW

SEQ ID NO: 721 - protein, 16 aa, synthetic construct
  source 1..16: note = VHHh Shuffle
CAAEVRNGSDYLPIDW

SEQ ID NO: 722 - protein, 12 aa, synthetic construct
  source 1..12: note = VHHh Shuffle
CAAFDGYSGSDW

SEQ ID NO: 723 - protein, 12 aa, synthetic construct
  source 1..12: note = VHHh Shuffle
CAAFDGYTGSDW

SEQ ID NO: 724 - protein, 13 aa, synthetic construct
  source 1..13: note = VHHh Shuffle
CAAQTEDSAQYIW

SEQ ID NO: 725 - protein, 13 aa, synthetic construct
  source 1..13: note = VHHh Shuffle
CAARRWIPPGPIW

SEQ ID NO: 726 - protein, 12 aa, synthetic construct
  source 1..12: note = VHHh Shuffle
CAKEDVGKPFDW

SEQ ID NO: 727 - protein, 12 aa, synthetic construct
  source 1..12: note = VHHh Shuffle
CAKQDVGKPFDW

SEQ ID NO: 728 - protein, 13 aa, synthetic construct
  source 1..13: note = VHHh Shuffle
CALRVRPYGQYDW

SEQ ID NO: 729 - protein, 14 aa, synthetic construct
  source 1..14: note = VHHh Shuffle HI
CAREDYYDSSGYSW

SEQ ID NO: 730 - protein, 20 aa, synthetic construct
  source 1..20: note = VHHh Shuffle HI
CAREGYYYDSSGYPYYFDYW

SEQ ID NO: 731 - protein, 20 aa, synthetic construct
  source 1..20: note = VHHh Shuffle HI
CARERRYYDSSGYPYYFDYW

SEQ ID NO: 732 - protein, 30 aa, synthetic construct
  source 1..30: note = VHHh Shuffle
CAREVGLYYYGSGSSSRRLLGRIDYYFDYW

SEQ ID NO: 733 - protein, 10 aa, synthetic construct
  source 1..10: note = VHHh Shuffle HI
CARWGPFDIW

SEQ ID NO: 734 - protein, 13 aa, synthetic construct
  source 1..13: note = VHHh Shuffle
CASAYNPGIGYDW

SEQ ID NO: 735 - protein, 16 aa, synthetic construct
  source 1..16: note = VHHh Shuffle
CATGPYRSYFARSYLW

SEQ ID NO: 736 - protein, 13 aa, synthetic construct
  source 1..13: note = VHHh Shuffle
CAVDLSGDAVYDW

SEQ ID NO: 737 - protein, 20 aa, synthetic construct
  source 1..20: note = VHHh Shuffle HI
CAVVAMRMVTTEGPDVLDVW

SEQ ID NO: 738 - protein, 11 aa, synthetic construct
  source 1..11: note = VHHh Shuffle
CARVDRDFDYW

SEQ ID NO: 739 - protein, 12 aa, synthetic construct
  source 1..12: note = VHHh Shuffle HI
CWRLGNDYFDYW

SEQ ID NO: 740 - protein, 9 aa, synthetic construct
  source 1..9: note = VHHh Shuffle
GTFSSIGMG

SEQ ID NO: 741 - protein, 9 aa, synthetic construct
  source 1..9: note = VHHh Shuffle HI
FTFSPSWMG

SEQ ID NO: 742 - protein, 6 aa, synthetic construct
  source 1..6: note = VHHh Shuffle HI
QTFNMG

SEQ ID NO: 743 - protein, 9 aa, synthetic construct
  source 1..9: note = VHHh Shuffle
FTFRRYDMG

SEQ ID NO: 744 - protein, 9 aa, synthetic construct
  source 1..9: note = VHHh Shuffle
STFSINAMG

SEQ ID NO: 745 - protein, 9 aa, synthetic construct
  source 1..9: note = VHHh Shuffle
RTFSMHAMG

SEQ ID NO: 746 - protein, 9 aa, synthetic construct
  source 1..9: note = VHHh Shuffle
STLSINAMG

SEQ ID NO: 747 - protein, 14 aa, synthetic construct
  source 1..14: note = VHHh Shuffle
VAAISWDGGATAYA

SEQ ID NO: 748 - protein, 13 aa, synthetic construct
  source 1..13: note = VHHh Shuffle HI
VATINEYGGRNYA

SEQ ID NO: 749 - protein, 13 aa, synthetic construct
  source 1..13: note = VHHh Shuffle HI
VAAIGSGGSTSYA

SEQ ID NO: 750 - protein, 11 aa, synthetic construct
  source 1..11: note = VHHh Shuffle
SAISGGLAYYA

SEQ ID NO: 751 - protein, 13 aa, synthetic construct
  source 1..13: note = VHHh Shuffle
AGITSSGGYTNYA

SEQ ID NO: 752 - protein, 13 aa, synthetic construct
  source 1..13: note = VHHh Shuffle
AAISWDGGATAYA

SEQ ID NO: 753 - protein, 12 aa, synthetic construct
  source 1..12: note = VHHh Shuffle
AGISRGGTTNYA